(19) United States
(12) Patent Application Publication
(10) Pub. No.: US 2008/0045514 A1
(43) Pub. Date: Feb. 21, 2008

(54) 3-Aryl-6-aryl-7H-[1,2,4]triazolo[3,4-b][1,3,4]thiadiazines and analogs as activators of caspases and inducers of apoptosis and the use thereof

(75) Inventors: Sui Xiong Cai, San Diego, CA (US); John A. Drewe, Carlsbad, CA (US); Han-Zhong Zhang, San Diego, CA (US); Shailaja Kasibhatla, San Diego, CA (US); Gisela Claassen, San Diego, CA (US); Nilantha Sudath Sirisoma, San Diego, CA (US); William Edward Kemnitzer, San Diego, CA (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Cytovia, Inc., San Diego, CA (US)

(21) Appl. No.: 11/879,517

(22) Filed: Jul. 18, 2007

Related U.S. Application Data
(60) Provisional application No. 60/831,458, filed on Jul. 18, 2006.
(60) Provisional application No. 60/879,538, filed on Jan. 10, 2007.

(51) Int. Cl. A61K 31/542; A61K 31/53; A61P 35/00; C07D 487/04; C07D 513/04

(52) U.S. Cl. 514/222.8; 514/243; 544/10; 544/184

(57) ABSTRACT

Disclosed are 3-aryl-6-aryl-7H-[1,2,4]triazolo[3,4-b][1,3,4]thiadiazines and analogs thereof, represented by the Formula I: 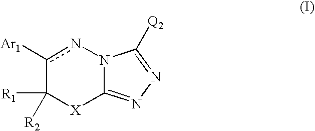
wherein Ar₁, Q₂, R₁, R₂, dashed line and X are defined herein. The present invention relates to the discovery that compounds having Formula I are activators of caspases and inducers of apoptosis. Therefore, the activators of caspases and inducers of apoptosis of this invention may be used to induce cell death in a variety of clinical conditions in which uncontrolled growth and spread of abnormal cells occurs.

### BACKGROUND OF THE INVENTION

1. Field of the Invention

This invention is in the field of medicinal chemistry. In particular, the invention relates to 3-aryl-6-aryl-7H-[1,2,4]triazolo[3,4-b][1,3,4]thiadiazines and analogs, and the discovery that these compounds are activators of caspases and inducers of apoptosis. The invention also relates to the use of these compounds as therapeutically effective anti-cancer agents.

2. Related Art

Organisms eliminate unwanted cells by a process variously known as regulated cell death, programmed cell death or apoptosis. Such cell death occurs as a normal aspect of animal development, as well as in tissue homeostasis and aging (Glucksmann, A., Biol. Rev. Cambridge Philos. Soc. 26:59-86 (1951); Glucksmann, A., Archives de Biologie 76:419-437 (1965); Ellis, et al., Dev. 112:591-603 (1991); Vaux, et al., Cell 76:777-779 (1994)). Apoptosis regulates cell number, facilitates morphogenesis, removes harmful or otherwise abnormal cells and eliminates cells that have already performed their function. Additionally, apoptosis occurs in response to various physiological stresses, such as hypoxia or ischemia (PCT published application WO96/20721).

There are a number of morphological changes shared by cells experiencing regulated cell death, including plasma and nuclear membrane blebbing, cell shrinkage (condensation of nucleoplasm and cytoplasm), organelle relocalization and compaction, chromatin condensation and production of apoptotic bodies (membrane enclosed particles containing intracellular material) (Orrenius, S., J. Internal Medicine 237:529-536 (1995)).

Apoptosis is achieved through an endogenous mechanism of cellular suicide (Wyllie, A. H., in Cell Death in Biology and Pathology, Bowen and Lockshin, eds., Chapman and Hall (1981), pp. 9-34). A cell activates its internally encoded suicide program as a result of either internal or external signals. The suicide program is executed through the activation of a carefully regulated genetic program (Wyllie, et al., Int. Rev. Cyt. 68:251 (1980); Ellis, et al., Ann. Rev. Cell Bio. 7:663 (1991)). Apoptotic cells and bodies are usually recognized and cleared by neighboring cells or macrophages before lysis. Because of this clearance mechanism, inflammation is not induced despite the clearance of great numbers of cells (Orrenius, S., J. Internal Medicine 237:529-536 (1995)).

It has been found that a group of proteases are a key element in apoptosis (see, e.g., Thornberry, Chemistry and Biology 5:R97-R103 (1998); Thornberry, British Med. Bull. 53:478-490 (1996)). Genetic studies in the nematode Caenorhabditis elegans revealed that apoptotic cell death involves at least 14 genes, 2 of which are the pro-apoptotic (death-promoting) ced (for cell death abnormal) genes, ced-3 and ced-4. CED-3 is homologous to interleukin 1 beta-converting enzyme, a cysteine protease, which is now called caspase-1. When these data were ultimately applied to mammals, and upon further extensive investigation, it was found that the mammalian apoptosis system appears to involve a cascade of caspases, or a system that behaves like a cascade of caspases. At present, the caspase family of cysteine proteases comprises 14 different members, and more may be discovered in the future. All known caspases are synthesized as zymogens that require cleavage at an aspartyl residue prior to forming the active enzyme. Thus, caspases are capable of activating other caspases, in the manner of an amplifying cascade.

Apoptosis and caspases are thought to be crucial in the development of cancer (Apoptosis and Cancer Chemotherapy, Hickman and Dive, eds., Humana Press (1999)). There is mounting evidence that cancer cells, while containing caspases, lack parts of the molecular machinery that activates the caspase cascade. This makes the cancer cells lose their capacity to undergo cellular suicide and the cells become cancerous. In the case of the apoptosis process, control points are known to exist that represent points for intervention leading to activation. These control points include the CED-9-BCL-like and CED-3-ICE-like gene family products, which are intrinsic proteins regulating the decision of a cell to survive or die and executing part of the cell death process itself, respectively (see, Schmitt, et al., Biochem. Cell. Biol. 75:301-314 (1997)). BCL-like proteins include BCL-xL and BAX-alpha, which appear to function upstream of caspase activation. BCL-xL appears to prevent activation of the apoptotic protease cascade, whereas BAX-alpha accelerates activation of the apoptotic protease cascade.

It has been shown that chemotherapeutic (anti-cancer) drugs can trigger cancer cells to undergo suicide by activating the dormant caspase cascade. This may be a crucial aspect of the mode of action of most, if not all, known anticancer drugs (Los, et al., Blood 90:3118-3129 (1997); Friesen, et al., Nat. Med. 2:574 (1996)). The mechanism of action of current antineoplastic drugs frequently involves an attack at specific phases of the cell cycle. In brief, the cell cycle refers to the stages through which cells normally progress during their lifetime. Normally, cells exist in a resting phase termed Gₒ. During multiplication, cells progress to a stage in which DNA synthesis occurs, termed S. Later, cell division, or mitosis occurs, in a phase called M. Antineoplastic drugs, such as cytosine arabinoside, hydroxyurea, 6-mercaptopurine, and methotrexate are S phase specific, whereas antineoplastic drugs, such as vincristine, vinblastine, and paclitaxel are M phase specific. Many slow growing tumors, e.g. colon cancers, exist primarily in the Gₒ phase, whereas rapidly proliferating normal tissues, for example bone marrow, exist primarily in the S or M phase. Thus, a drug like 6-mercaptopurine can cause bone marrow toxicity while remaining ineffective for a slow growing tumor. Further aspects of the chemotherapy of neoplastic diseases are known to those skilled in the art (see, e.g., Hardman, et al., eds., Goodman and Gilman's The Pharmacological Basis of Therapeutics, Ninth Edition, McGraw-Hill, New York (1996), pp. 1225-1287). Thus, it is clear that the possibility exists for the activation of the caspase cascade, although the exact mechanisms for doing so are not clear at this point. It is equally clear that insufficient activity of the caspase cascade and consequent apoptotic events are implicated in various types of cancer. The development of caspase cascade activators and inducers of apoptosis is a highly desirable goal in the development of therapeutically effective antineoplastic agents. Moreover, since autoimmune disease and certain degenerative diseases also involve the proliferation of abnormal cells, therapeutic treatment for these diseases could also involve the enhancement of the apoptotic process through the administration of appropriate caspase cascade activators and inducers of apoptosis.

C-Myc is a proto-oncogene and encodes the c-Myc transcription factor. Physiologically, cMyc expression correlates with cell proliferation in various cells and tissues of the body. CMyc is implicated in various biological processes including cell growth, proliferation, loss of differentiation and apoptosis. Deregulated expression of c-Myc occurs in a wide range of cancers and is often associated with poor prognosis suggesting an important role for this oncogene in tumor progression. Initially it was discovered in Burkitt's lymphoma as causative for the progression of the disease due to a translocation between chromosome 8 and the antibody-containing genes. More recently, cMyc has been detected in a wide range of cancers that include breast, colon, cervical, small-cell lung carcinomas, osteocarcomas, glioblastomas, melanoma and myeloid leukemias (Nesbit, C E et al. Oncogene, 18, 3004-3016 (1999); Blackwood, E. M et al. Science 251, 1211-1217 (1991); Mo H. & Henriksson M. PNAS, 103: 6344-6349 (2006)) Inactivation of c-Myc was found to cause tumor regression with rapid proliferation arrest and apoptosis in hematopoietic malignancies and osteosarcoma. Therefore inactivating cMyc or downstream targets of cMyc may provide important therapeutic advantages.

Nadkarni et al. (Arzneimittel-Forschung 51:569-573 (2001)) reported the synthesis of a series of 3,6-disubstituted-7H-s-triazolo(3,4-b)(1,3,4)thiadiazines I (X═F, Cl, R═H, 2-F, 3-F, 4-F, 3-CF3, etc.) by the condensation of the appropriate 3-substituted-4-amino-5-mercapto(1,2,4)triazoles II (X═F, Cl) with substituted phenacyl bromides in alcohol medium. These compounds have been studied for their in vivo anthelmintic activity in albino mice. A number of compounds showed promising activity when given by the oral route.

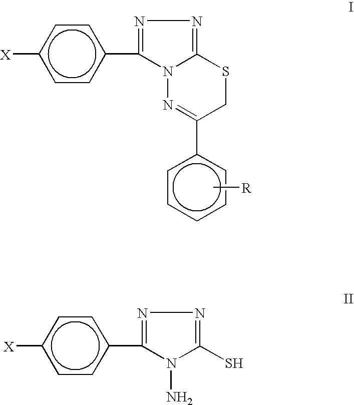

### SUMMARY OF THE INVENTION

The present invention is related to the discovery that 3-aryl-6-aryl-7H-[1,2,4]triazolo[3,4-b][1,3,4]thiadiazines and analogs, as represented in Formulae I-V, are activators of the caspase cascade and inducers of apoptosis. Thus, an aspect of the present invention is directed to the use of compounds of Formulae I-V as inducers of apoptosis.

A second aspect of the present invention is to provide a method for treating, preventing or ameliorating neoplasia and cancer by administering a compound of one of the Formulae I-V to a mammal in need of such treatment.

Many of the compounds within the scope of the present invention are novel compounds. Therefore, a third aspect of the present invention is to provide novel compounds of Formulae I-V, and to also provide for the use of these novel compounds for treating, preventing or ameliorating neoplasia and cancer.

A fourth aspect of the present invention is to provide a pharmaceutical composition useful for treating disorders responsive to the induction of apoptosis, containing an effective amount of a compound of one of the Formulae I-V in admixture with one or more pharmaceutically acceptable carriers or diluents.

A fifth aspect of the present invention is directed to methods for the preparation of novel compounds of Formulae I-V.

### DETAILED DESCRIPTION OF THE INVENTION

The present invention arises out of the discovery that 3-aryl-6-aryl-7H-[1,2,4]triazolo[3,4-b][1,3,4]thiadiazines and analogs, as represented in Formulae I-V, are potent and highly efficacious activators of the caspase cascade and inducers of apoptosis. Therefore, compounds of Formulae I-V are useful for treating disorders responsive to induction of apoptosis.

Specifically, compounds of the present invention are represented by Formula I: 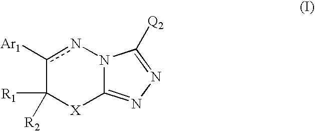
or pharmaceutically acceptable salts or prodrugs or tautomers thereof, wherein:

the dashed line signifies an optional double bond wherein when the dashed line represents a single bond, there is a hydrogen on the nitrogen and neighboring carbon;

Ar₁ is an optionally substituted aryl or optionally substituted heteroaryl;

Q₂ is an optionally substituted alkyl, carbocyclic, heterocyclic, aryl or heteroaryl;

R₁ and R₂ independently are hydrogen, halo, optionally substituted amino, optionally substituted alkoxy, optionally substituted C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, carboxyalkyl, nitro, cyano, acylamido, hydroxy, thiol, acyloxy, azido, carboxy, carbonylamido or optionally substituted alkylthiol; or R₁ and R₂ are combined as ═O; and

X is S, O or NR₃, wherein R₃ is hydrogen or an optionally substituted alkyl or aryl.

Preferred compounds of formula I include compounds wherein the dashed line represents a double bond.

Other preferred compounds of Formula I include compounds wherein Ar₁ is phenyl, naphthyl, pyridyl, quinolyl, isoquinolyl, isoxazolyl, pyrazolyl, imidazolyl, thienyl, furyl or pyrrolyl, each of which is optionally substituted. Another group of preferred compounds are wherein Q₂ is an optionally substituted aryl or heteroaryl. More preferably, Ar₁ and Q₂ are phenyl or pyridyl. Another group of preferred compounds of Formula I include compounds wherein R₁ and R₂ are hydrogen. Another group of preferred compounds of Formula I include compounds wherein R₃ is hydrogen. Another group of preferred compounds of Formula I include compounds wherein X is S or O. Another group of preferred compounds of Formula I include compounds wherein X is S.

One group of preferred compounds of the present invention are represented by Formulae II-IV:

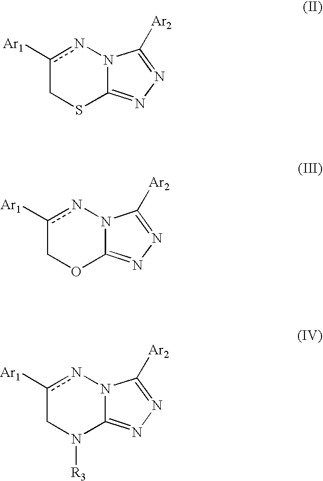

or pharmaceutically acceptable salts, prodrugs or tautomers thereof, wherein:

the dashed line signifies an optional double bond;

Ar₁ and Ar₂ independently are optionally substituted aryl or optionally substituted heteroaryl; and

R₃ is hydrogen or an optionally substituted alkyl or aryl.

Preferred compounds of formulae II-IV include compounds wherein the dashed line represents a double bond.

Other preferred compounds of Formulae II-IV include compounds wherein Ar₁ and Ar₂ are phenyl, naphthyl, pyridyl, quinolyl, isoquinolyl, isoxazolyl, pyrazolyl, imidazolyl, thienyl, furyl or pyrrolyl, each of which is optionally substituted. More preferably, Ar₁ and Ar₂ are phenyl or pyridyl. Another group of preferred compounds of Formula IV include compounds wherein R₃ is hydrogen.

Another group of preferred compounds of the present invention are represented by Formula V:

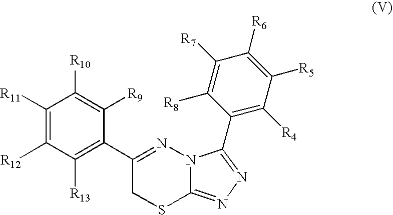

or pharmaceutically acceptable salts, prodrugs or tautomers thereof, wherein:

R₄-R₁₃ independently are hydrogen, halo, amino, di(C₁₋₁₀ alkyl)amino, alkoxy, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, hydroxyalkoxy, aminoalkyl, aminoalkoxy, carboxyalkyl, nitro, cyano, acylamido, hydroxy, thiol, acyloxy, azido, carboxy, carbonylamido, alkylsulfonyl, aminosulfonyl, dialkylaminosulfonyl, alkylsulfiniyl, or alkylthiol; or

R₄ and R₅, or R₅ and R₆, or R₆ and R₇, or R₇ and R₈, or R₉ and R₁₀, or R₁₀ and R₁₁, or R₁₁ and R₁₂, or R₁₂ and R₁₃, taken together with the atoms to which they are attached to form an aryl, heteroaryl, partially saturated carbocyclic or partially saturated heterocyclic group, wherein said group is optionally substituted.

Preferred are compounds of Formula V, wherein R₄ and R₅, or R₅ and R₆, or R₆ and R₇, or R₇ and R₈, or R₉ and R₁₀, or R₁₀ and R₁₁, or R₁₁ and R₁₂, or R₁₂ and R₁₃, taken together to form a structure selected from the group consisting of —OCH₂O—, —(CH₂)₃—, —(CH₂)₄—, —OCH₂CH₂O—, —CH₂N(R₁₄)CH₂—, —CH₂CH₂N(R₁₄)CH₂—, —CH₂N(R₁₄)CH₂CH₂—, —N(R₁₄)—CH═CH—, —CH═CH—N(R₁₄)—, —N(R₁₄)CH₂—CH₂—, —CH₂—CH₂—N(R₁₄)—, —N(R₁₄)—CH═N—, —N═CH—N(R₁₄)—, —O—CH═CH—, —CH═CH—O—, —S—CH═CH—, —CH═CH—S—, —N—C(═O)—O—, —N—CH₂—CH₂—N— and —N═CH—CH═N—, wherein R₁₄ is hydrogen, C₁₋₁₀ alkyl, haloalkyl, aryl, fused aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl or aminoalkyl.

Exemplary preferred compounds of Formulae I-V that may be employed in the method of the invention include, without limitation:
3-(4-Methylphenyl)-6-(3,4-dimethoxyphenyl)-7H-[1,2,4]triazolo[3,4-b][1,3,4]thiadiazine;
3-(4-Methylphenyl)-6-(4-ethoxyphenyl)-7H-[1,2,4]triazolo[3,4-b][1,3,4]thiadiazine;
3-(4-Methylphenyl)-6-(3,4-methylenedioxyphenyl)-7H-[1,2,4]triazolo[3,4-b][1,3,4]thiadiazine;
3-(4-Chlorophenyl)-6-(4-methoxyphenyl)-7H-[1,2,4]triazolo[3,4-b][1,3,4]thiadiazine;
3-(4-Chlorophenyl)-6-(3,4-dimethoxyphenyl)-7H-[1,2,4]triazolo[3,4-b][1,3,4]thiadiazine;
3-(4-Chlorophenyl)-6-(4-ethoxyphenyl)-7H-[1,2,4]triazolo[3,4-b][1,3,4]thiadiazine;
3-(4-Chlorophenyl)-6-(3,4-methylenedioxyphenyl)-7H-[1,2,4]triazolo[3,4-b][1,3,4]thiadiazine;
3-(4-Methoxyphenyl)-6-(2,5-dimethoxyphenyl)-7H-[1,2,4]triazolo[3,4-b][1,3,4]thiadiazine;
3-(4-Methoxyphenyl)-6-(4-ethoxyphenyl)-7H-[1,2,4]triazolo[3,4-b][1,3,4]thiadiazine;
3-(4-Methoxyphenyl)-6-(3,4-methylenedioxyphenyl)-7H-[1,2,4]triazolo[3,4-b][1,3,4]thiadiazine;
3-(2-Methylphenyl)-6-(3,4-dimethoxyphenyl)-7H-[1,2,4]triazolo[3,4-b][1,3,4]thiadiazine;
3-(2-Methylphenyl)-6-(4-ethoxyphenyl)-7H-[1,2,4]triazolo[3,4-b][1,3,4]thiadiazine;
3-(2-Methylphenyl)-6-(3,4-methylenedioxyphenyl)-7H-[1,2,4]triazolo[3,4-b][1,3,4]thiadiazine;
3-(2-Chlorophenyl)-6-(3,4-dimethoxyphenyl)-7H-[1,2,4]triazolo[3,4-b][1,3,4]thiadiazine;
3-(2-Chlorophenyl)-6-(4-ethoxyphenyl)-7H-[1,2,4]triazolo[3,4-b][1,3,4]thiadiazine;
3-(2-Chlorophenyl)-6-(3,4-methylenedioxyphenyl)-7H-[1,2,4]triazolo[3,4-b][1,3,4]thiadiazine;
3-(2-Chlorophenyl)-6-(4-bromophenyl)-7H-[1,2,4]triazolo[3,4-b][1,3,4]thiadiazine;
3-(3-Chlorophenyl)-6-(4-methoxyphenyl)-7H-[1,2,4]triazolo[3,4-b][1,3,4]thiadiazine;
3-(3-Chlorophenyl)-6-(3,4-methylenedioxyphenyl)-7H-[1,2,4]triazolo[3,4-b][1,3,4]thiadiazine;
3-(3-Methylphenyl)-6-(3,4-methylenedioxyphenyl)-7H-[1,2,4]triazolo[3,4-b][1,3,4]thiadiazine;
3-(3-Methylphenyl)-6-(2-naphthyl)-7H-[1,2,4]triazolo[3,4-b][1,3,4]thiadiazine;
3-(2-Methoxyphenyl)-6-(3,4-methylenedioxyphenyl)-7H-[1,2,4]triazolo[3,4-b][1,3,4]thiadiazine;
3-(2-Methoxyphenyl)-6-(4-bromophenyl)-7H-[1,2,4]triazolo[3,4-b][1,3,4]thiadiazine;
3-(3-Methoxyphenyl)-6-(4-methoxyphenyl)-7H-[1,2,4]triazolo[3,4-b][1,3,4]thiadiazine;
3-(3-Methoxyphenyl)-6-(3,4-dimethoxyphenyl)-7H-[1,2,4]triazolo[3,4-b][1,3,4]thiadiazine;
3-(3-Methoxyphenyl)-6-(4-ethoxyphenyl)-7H-[1,2,4]triazolo[3,4-b][1,3,4]thiadiazine;
3-(3-Methoxyphenyl)-6-(3,4-methylenedioxyphenyl)-7H-[1,2,4]triazolo[3,4-b][1,3,4]thiadiazine;
3-(3-Methoxyphenyl)-6-(2-naphthyl)-7H-[1,2,4]triazolo[3,4-b][1,3,4]thiadiazine;
3-(3-Ethoxyphenyl)-6-(3,4-dihydroxyphenyl)-7H-[1,2,4]triazolo[3,4-b][1,3,4]thiadiazine;
3-(3-Ethoxyphenyl)-6-(4-methoxyphenyl)-7H-[1,2,4]triazolo[3,4-b][1,3,4]thiadiazine;
3-(3-Ethoxyphenyl)-6-(3,4-methylenedioxyphenyl)-7H-[1,2,4]triazolo[3,4-b][1,3,4]thiadiazine;
3-(4-Ethoxyphenyl)-6-(3,4-dihydroxyphenyl)-7H-[1,2,4]triazolo[3,4-b][1,3,4]thiadiazine;
3-(2,4-Dichlorophenyl)-6-(3,4-dimethoxyphenyl)-7H-[1,2,4]triazolo[3,4-b][1,3,4]thiadiazine;
3-(2,4-Dichlorophenyl)-6-(4-ethoxyphenyl)-7H-[1,2,4]triazolo[3,4-b][1,3,4]thiadiazine;
3-(2,4-Dichlorophenyl)-6-(3,4-methylenedioxyphenyl)-7H-[1,2,4]triazolo[3,4-b][1,3,4]thiadiazine;
3-(2,4-Dichlorophenyl)-6-phenyl-7H-[1,2,4]triazolo[3,4-b][1,3,4]thiadiazine;
3-(2-Methoxyphenyl)-6-(3,4-dimethoxyphenyl)-7H-[1,2,4]triazolo[3,4-b][1,3,4]thiadiazine;
3-(3,4,5-Trimethoxyphenyl)-6-(3,4-dimethoxyphenyl)-7H-[1,2,4]triazolo[3,4-b][1,3,4]thiadiazine;
3-(3,4,5-Trimethoxyphenyl)-6-(3,4-methylenedioxyphenyl)-7H-[1,2,4]triazolo[3,4-b][1,3,4]thiadiazine;
3-(3,4-Dimethoxyphenyl)-6-(3,4-dimethoxyphenyl)-7H-[1,2,4]triazolo[3,4-b][1,3,4]thiadiazine;
3-(3,4-Dimethoxyphenyl)-6-(3,4-methylenedioxyphenyl)-7H-[1,2,4]triazolo[3,4-b][1,3,4]thiadiazine;
3-Phenyl-6-(3,4-methylenedioxyphenyl)-7H-[1,2,4]triazolo[3,4-b][1,3,4]thiadiazine;
3-(2-Fluorophenyl)-6-(3,4-methylenedioxyphenyl)-7H-[1,2,4]triazolo[3,4-b][1,3,4]thiadiazine;
3-(3-Isopropyloxyphenyl)-6-(3,4-methylenedioxyphenyl)-7H-[1,2,4]triazolo[3,4-b][1,3,4]thiadiazine;
3-(3,4,5-Trimethoxyphenyl)-6-phenyl-7H-[1,2,4]triazolo[3,4-b][1,3,4]thiadiazine;
3-(3,4,5-Trimethoxyphenyl)-6-(4-ethoxyphenyl)-7H-[1,2,4]triazolo[3,4-b][1,3,4]thiadiazine;
3-(3,4,5-Trimethoxyphenyl)-6-(4-bromophenyl)-7H-[1,2,4]triazolo[3,4-b][1,3,4]thiadiazine;
3-(3,4,5-Trimethoxyphenyl)-6-(4-fluorophenyl)-7H-[1,2,4]triazolo[3,4-b][1,3,4]thiadiazine;
3-(3,4,5-Trimethoxyphenyl)-6-(4-chlorophenyl)-7H-[1,2,4]triazolo[3,4-b][1,3,4]thiadiazine;
3-(3,4,5-Trimethoxyphenyl)-6-(3-bromophenyl)-7H-[1,2,4]triazolo[3,4-b][1,3,4]thiadiazine;
3-(3,4,5-Trimethoxyphenyl)-6-(2-naphthyl)-7H-[1,2,4]triazolo[3,4-b][1,3,4]thiadiazine;
3-(3,4,5-Trimethoxyphenyl)-6-(3,4-dihydroxyphenyl)-7H-[1,2,4]triazolo[3,4-b][1,3,4]thiadiazine;
3-(3,4,5-Trimethoxyphenyl)-6-(4-methoxyphenyl)-7H-[1,2,4]triazolo[3,4-b][1,3,4]thiadiazine;
3-(2-Methoxyphenyl)-6-(4-ethoxyphenyl)-7H-[1,2,4]triazolo[3,4-b][1,3,4]thiadiazine;
3-(2-Chlorophenyl)-6-(4-methoxyphenyl)-7H-[1,2,4]triazolo[3,4-b][1,3,4]thiadiazine;
3-(3-Methoxyphenyl)-6-(3,4-dihydroxyphenyl)-7H-[1,2,4]triazolo[3,4-b][1,3,4]thiadiazine;
3-(3-Chlorophenyl)-6-(4-ethoxyphenyl)-7H-[1,2,4]triazolo[3,4-b][1,3,4]thiadiazine;
3-(3-Methoxyphenyl)-6-(4-bromophenyl)-7H-[1,2,4]triazolo[3,4-b][1,3,4]thiadiazine;
3-(3-Ethoxyphenyl)-6-(4-ethoxyphenyl)-7H-[1,2,4]triazolo[3,4-b][1,3,4]thiadiazine;
3-(4-Ethoxyphenyl)-6-(3,4-methylenedioxyphenyl)-7H-[1,2,4]triazolo[3,4-b][1,3,4]thiadiazine;
3-(3-Bromophenyl)-6-(4-methoxyphenyl)-7H-[1,2,4]triazolo[3,4-b][1,3,4]thiadiazine;
3-Ethyl-6-(4-methoxyphenyl)-7H-[1,2,4]triazolo[3,4-b][1,3,4]thiadiazine;
3-(3-Isopropyloxyphenyl)-6-(4-methoxyphenyl)-7H-[1,2,4]triazolo[3,4-b][1,3,4]thiadiazine;
3-(2-Fluorophenyl)-6-(4-methoxyphenyl)-7H-[1,2,4]triazolo[3,4-b][1,3,4]thiadiazine;
3-Cyclohexyl-6-(3,4-methylenedioxyphenyl)-7H-[1,2,4]triazolo[3,4-b][1,3,4]thiadiazine;
3-(2-Methylphenyl)-6-(4-methoxyphenyl)-7H-[1,2,4]triazolo[3,4-b][1,3,4]thiadiazine;
3-(3-Methylphenyl)-6-(4-ethoxyphenyl)-7H-[1,2,4]triazolo[3,4-b][1,3,4]thiadiazine;
3-(4-Fluorophenyl)-6-(4-ethoxyphenyl)-7H-[1,2,4]triazolo[3,4-b][1,3,4]thiadiazine;
3-(2-Methylphenyl)-6-(4-methoxyphenyl)-7H-[1,2,4]triazolo[3,4-b][1,3,4]thiadiazine;
3-(3-Methylphenyl)-6-(4-methoxyphenyl)-7H-[1,2,4]triazolo[3,4-b][1,3,4]thiadiazine;
3-Cyclohexyl-6-(4-methoxyphenyl)-7H-[1,2,4]triazolo[3,4-b][1,3,4]thiadiazine;
3-(4-Fluorophenyl)-6-(4-methoxyphenyl)-7H-[1,2,4]triazolo[3,4-b][1,3,4]thiadiazine;
3-(Furan-2-yl)-6-(4-methoxyphenyl)-7H-[1,2,4]triazolo[3,4-b][1,3,4]thiadiazine;
3-(4-Methylphenyl)-6-(4-methoxyphenyl)-7H-[1,2,4]triazolo[3,4-b][1,3,4]thiadiazine;
3-(1,4,5,6-Tetrahydrocyclopenta[c]pyrazol-3-yl)-6-(4-methoxyphenyl)-7H-[1,2,4]triazolo[3,4-b][1,3,4]thiadiazine;
3-(1,4,5,6-Tetrahydrocyclopenta[c]pyrazol-3-yl)-6-(3,4-methylenedioxyphenyl)-7H-[1,2,4]triazolo[3,4-b][1,3,4]thiadiazine;
3-(2-Methoxyphenyl)-6-(4-nitrophenyl)-7H-[1,2,4]triazolo[3,4-b][1,3,4]thiadiazine;
3-(4-Methylphenyl)-6-(4-difluoromethoxyphenyl)-7H-[1,2,4]triazolo[3,4-b][1,3,4]thiadiazine;
3-(4-Bromophenyl)-6-(4-ethoxyphenyl)-7H-[1,2,4]triazolo[3,4-b][1,3,4]thiadiazine;
3-(2-Bromophenyl)-6-(4-ethoxyphenyl)-7H-[1,2,4]triazolo[3,4-b][1,3,4]thiadiazine;
3-(4-Methoxyphenyl)-6-(4-methoxyphenyl)-7H-[1,2,4]triazolo[3,4-b][1,3,4]thiadiazine;
3-(3-Fluorophenyl)-6-(4-methoxyphenyl)-7H-[1,2,4]triazolo[3,4-b][1,3,4]thiadiazine;
3-(2-Bromophenyl)-6-(4-methoxyphenyl)-7H-[1,2,4]triazolo[3,4-b][1,3,4]thiadiazine;
3-(4-Bromophenyl)-6-(4-methoxyphenyl)-7H-[1,2,4]triazolo[3,4-b][1,3,4]thiadiazine;
3-(4-Methoxyphenyl)-6-(4-methylphenyl)-7H-[1,2,4]triazolo[3,4-b][1,3,4]thiadiazine;
3-(3-Fluorophenyl)-6-(4-ethylphenyl)-7H-[1,2,4]triazolo[3,4-b][1,3,4]thiadiazine;
6-(5-Bromothiophen-2-yl)-3-(3-methoxyphenyl)-7H-[1,2,4]triazolo[3,4-b][1,3,4]thiadiazine;
6-(4-Methylphenyl)-3-phenyl-7H-[1,2,4]triazolo[3,4-b][1,3,4]thiadiazine;
6-(4-Chlorophenyl)-3-(2-methoxyphenyl)-7H-[1,2,4]triazolo[3,4-b][1,3,4]thiadiazine;
3-(2-Methoxyphenyl)-6-phenyl-7H-[1,2,4]triazolo[3,4-b][1,3,4]thiadiazine;
3-(2,4-Dichlorophenyl)-6-(4-methylphenyl)-7H-[1,2,4]triazolo[3,4-b][1,3,4]thiadiazine;
3-(2,3-Dichlorophenyl)-6-(4-methoxyphenyl)-7H-[1,2,4]triazolo[3,4-b][1,3,4]thiadiazine;
3-(2,4-Dichlorophenyl)-6-(4-methoxyphenyl)-7H-[1,2,4]triazolo[3,4-b][1,3,4]thiadiazine;
3-(2,4-Dichlorophenyl)-6-(3-methoxyphenyl)-7H-[1,2,4]triazolo[3,4-b][1,3,4]thiadiazine;
6-(4-Fluorophenyl)-3-(2-methoxyphenyl)-7H-[1,2,4]triazolo[3,4-b][1,3,4]thiadiazine;
6-(3,4-Dichlorophenyl)-3-(2-methoxyphenyl)-7H-[1,2,4]triazolo[3,4-b][1,3,4]thiadiazine;
3-(3,5-dimethoxyphenyl)-6-(3,4-methylenedioxyphenyl)-7H-[1,2,4]triazolo[3,4-b][1,3,4]thiadiazine;
3-(3-Chloro-4,5-dimethoxyphenyl)-6-(3,4-methylenedioxyphenyl)-7H-[1,2,4]triazolo[3,4-b][1,3,4]thiadiazine;
3-(3-Chloro-4,5-dimethoxyphenyl)-6-(4-methoxyphenyl)-7H-[1,2,4]triazolo[3,4-b][1,3,4]thiadiazine;
3-(4-Chlorophenyl)-6-(4-hydroxyphenyl)-7H-[1,2,4]triazolo[3,4-b][1,3,4]thiadiazine;
6-(4-Hydroxyphenyl)-3-(2-methoxyphenyl)-7H-[1,2,4]triazolo[3,4-b][1,3,4]thiadiazine;
3-(2-Methoxyphenyl)-6-(4-methylphenyl)-7H-[1,2,4]triazolo[3,4-b][1,3,4]thiadiazine;
3-(2-Methoxyphenyl)-6-(4-(2-dimethylaminoethoxy)phenyl)-7H-[1,2,4]triazolo[3,4-b][1,3,4]thiadiazine;
6-(4-(2-Hydroxyethoxy)phenyl)-3-(2-methoxyphenyl)-7H-[1,2,4]triazolo[3,4-b][1,3,4]thiadiazine;
3-(2-Chlorophenyl)-6-(4-methylphenyl)-7H-[1,2,4]triazolo[3,4-b][1,3,4]thiadiazine;
3-(3,5-Dimethoxyphenyl)-6-(4-methylphenyl)-7H-[1,2,4]triazolo[3,4-b][1,3,4]thiadiazine;
3-(3,5-Dimethoxy-4-hydroxyphenyl)-6-(4-methylphenyl)-7H-[1,2,4]triazolo[3,4-b][1,3,4]thiadiazine;
3-(3,4-Dimethoxyphenyl)-6-(4-methylphenyl)-7H-[1,2,4]triazolo[3,4-b][1,3,4]thiadiazine;
3-(2,4-Dimethoxyphenyl)-6-(4-methylphenyl)-7H-[1,2,4]triazolo[3,4-b][1,3,4]thiadiazine;
3-(3,5-Dimethoxy-4-(2-dimethylaminoethoxy)phenyl)-6-(4-methylphenyl)-7H-[1,2,4]triazolo[3,4-b][1,3,4]thiadiazine;
3-(3,4,5-Trimethoxyphenyl)-6-(4-methylphenyl)-7H-[1,2,4]triazolo[3,4-b][1,3,4]thiadiazine;
3-(2,4-Dimethoxyphenyl)-6-(4-methoxyphenyl)-7H-[1,2,4]triazolo[3,4-b][1,3,4]thiadiazine;
3-(2-Ethoxyphenyl)-6-(4-methylphenyl)-7H-[1,2,4]triazolo[3,4-b][1,3,4]thiadiazine;
3-(3,5-Dimethoxyphenyl)-6-(4-methoxyphenyl)-7H-[1,2,4]triazolo[3,4-b][1,3,4]thiadiazine;
6-(4-Diethylaminophenyl)-3-(3,4,5-trimethoxyphenyl)-7H-[1,2,4]triazolo[3,4-b][1,3,4]thiadiazine;
6-(4-Diethylaminophenyl)-3-(3,4-dimethoxyphenyl)-7H-[1,2,4]triazolo[3,4-b][1,3,4]thiadiazine;
6-(4-Diethylaminophenyl)-3-(2-methoxyphenyl)-7H-[1,2,4]triazolo[3,4-b][1,3,4]thiadiazine;
3-(2-Methoxyphenyl)-6-(4-(pyrrolidin-1-yl)phenyl)-7H-[1,2,4]triazolo[3,4-b][1,3,4]thiadiazine;
3-(2-Methoxyphenyl)-6-(4-morpholinophenyl)-7H-[1,2,4]triazolo[3,4-b][1,3,4]thiadiazine;
6-(4-Diethylaminophenyl)-3-(3,5-dimethoxyphenyl)-7H-[1,2,4]triazolo[3,4-b][1,3,4]thiadiazine;
6-(4-Diethylaminophenyl)-3-(2,4-dimethoxyphenyl)-7H-[1,2,4]triazolo[3,4-b][1,3,4]thiadiazine;
3-(3,4-Dimethoxyphenyl)-6-(4-nitrophenyl)-7H-[1,2,4]triazolo[3,4-b][1,3,4]thiadiazine;
6-(4-Aminophenyl)-3-(3,4-dimethoxyphenyl)-7H-[1,2,4]triazolo[3,4-b][1,3,4]thiadiazine;
6-(4-Dimethylminophenyl)-3-(3,4-dimethoxyphenyl)-7H-[1,2,4]triazolo[3,4-b][1,3,4]thiadiazine;
6-(4-Azidophenyl)-3-(3,4-dimethoxyphenyl)-7H-[1,2,4]triazolo[3,4-b][1,3,4]thiadiazine;
6-(4-Acetamidophenyl)-3-(3,4-dimethoxyphenyl)-7H-[1,2,4]triazolo[3,4-b][1,3,4]thiadiazine;
6-(2,3-Dihydro-2-oxobenzo[d]oxazol-6-yl)-3-(2,4-Dimethoxyphenyl)-7H-[1,2,4]triazolo[3,4-b][1,3,4]thiadiazine;
6-(2,3-Dihydro-2-oxobenzo[d]oxazol-6-yl)-3-(3,5-dimethoxyphenyl)-7H-[1,2,4]triazolo[3,4-b][1,3,4]thiadiazine;
6-(2,3-Dihydro-2-oxobenzo[d]oxazol-6-yl)-3-(3,4-dimethoxyphenyl)-7H-[1,2,4]triazolo[3,4-b][1,3,4]thiadiazine;
3-(2-Methoxyphenyl)-6-(4-methoxyphenyl)-[1,2,4]triazolo[4,3-b][1,2,4]triazin-7(8H)-one;
3-(3,4,5-Trimethoxyphenyl)-6-(4-methylphenyl)-1H-[1,2,4]triazolo[4,3-b][1,2,4]triazin-7-one;
3-(2-Methoxyphenyl)-6-(4-methylphenyl)-1H-[1,2,4]triazolo[4,3-b][1,2,4]triazin-7-one;
3-(2-Methoxyphenyl)-6-(2,4-dimethylphenyl)-7H-[1,2,4]triazolo[3,4-b][1,3,4]thiadiazine;
3-(2-Methoxyphenyl)-6-(3,4-dimethylphenyl)-7H-[1,2,4]triazolo[3,4-b][1,3,4]thiadiazine;
6-(2-Methoxy-4-methylphenyl)-3-(2-methoxyphenyl)-7H-[1,2,4]triazolo[3,4-b][1,3,4]thiadiazine;
6-(2,3,4-Trimethoxyphenyl)-3-(2-methoxyphenyl)-7H-[1,2,4]triazolo[3,4-b][1,3,4]thiadiazine;
6-(2,3-Dihydrobenzo[b][1,4]dioxin-6-yl)-3-(2-methoxyphenyl)-7H-[1,2,4]triazolo[3,4-b][1,3,4]thiadiazine;
6-(4-Isopropylphenyl)-3-(2-methoxyphenyl)-7H-[1,2,4]triazolo[3,4-b][1,3,4]thiadiazine;
6-(3-Methoxy-4-nitrophenyl)-3-(2-methoxyphenyl)-7H-[1,2,4]triazolo[3,4-b][1,3,4]thiadiazine;
3-(2-Methoxyphenyl)-6-(4-methoxyphenyl)-7H-[1,2,4]triazolo[3,4-b]-[1,3,4]thiadiazine;
6-(2,3-Dihydro-2-oxobenzo[d]oxazol-6-yl)-3-(2-methoxyphenyl)-7H-[1,2,4]triazolo[3,4-b][1,3,4]thiadiazine;
6-(4-(Trifluoromethyl)phenyl)-3-(2-methoxyphenyl)-7H-[1,2,4]triazolo[3,4-b][1,3,4]thiadiazine;
6-(5-Chloro-2-methoxy-4-methylphenyl)-3-(2-methoxyphenyl)-7H-[1,2,4]triazolo[3,4-b][1,3,4]thiadiazine;
3-(2-Methoxyphenyl)-6-(4-(trifluoromethoxy)phenyl)-7H-[1,2,4]triazolo[3,4-b][1,3,4]thiadiazine;
6-(4-tert-Butylphenyl)-3-(2-methylphenyl)-7H-[1,2,4]triazolo[3,4-b][1,3,4]thiadiazine;
6-(4-(N,N-Diethylaminosulfonyl)phenyl)-3-(2-methylphenyl)-7H-[1,2,4]triazolo[3,4-b][1,3,4]thiadiazine;
6-(4-Methyl-3-nitrophenyl)-3-(2-methylphenyl)-7H-[1,2,4]triazolo[3,4-b][1,3,4]thiadiazine;
3-(2-Methoxyphenyl)-6-(4-methyl-3-nitrophenyl)-7H-[1,2,4]triazolo[3,4-b][1,3,4]thiadiazine;
6-(4-Chloro-3-nitrophenyl)-3-(2-methoxyphenyl)-7H-[1,2,4]triazolo[3,4-b][1,3,4]thiadiazine;
6-(4-Acetamido-3-nitrophenyl)-3-(2-methoxyphenyl)-7H-[1,2,4]triazolo[3,4-b][1,3,4]thiadiazine;
6-(4-Amino-3-methoxyphenyl)-3-(2-methoxyphenyl)-7H-[1,2,4]triazolo[3,4-b][1,3,4]thiadiazine;
6-(3-Amino-4-methylphenyl)-3-(2-methoxyphenyl)-7H-[1,2,4]triazolo[3,4-b][1,3,4]thiadiazin;
6-(3-Amino-4-chlorophenyl)-3-(2-methoxyphenyl)-7H-[1,2,4]triazolo[3,4-b][1,3,4]thiadiazine;
6-(3,4-Diaminophenyl)-3-(2-methoxyphenyl)-7H-[1,2,4]triazolo[3,4-b][1,3,4]thiadiazine;
6-(3-Amino-4-methylphenyl)-3-(2-methylphenyl)-7H-[1,2,4]triazolo[3,4-b][1,3,4]thiadiazine;
3-(5-Chloro-2-methoxyphenyl)-6-(4-methylphenyl)-7H-[1,2,4]triazolo[3,4-b][1,3,4]thiadiazine;
3-(2-Methoxyphenyl)-6-(3-methoxylphenyl)-7H-[1,2,4]triazolo[3,4-b][1,3,4]thiadiazine;
3-(2-Methoxyphenyl)-6-(2-methoxylphenyl)-7H-[1,2,4]triazolo[3,4-b][1,3,4]thiadiazine;
3-(2,3-Dimethoxyphenyl)-6-(4-methylphenyl)-7H-[1,2,4]triazolo[3,4-b][1,3,4]thiadiazine;
6-(4-Aminophenyl)-3-(3,4,5-trimethoxyphenyl)-7H-[1,2,4]triazolo[3,4-b][1,3,4]thiadiazine;
6-(4-Dimethylaminophenyl)-3-(3,4,5-trimethoxyphenyl)-7H-[1,2,4]triazolo[3,4-b][1,3,4]thiadiazine;
3-(2-Fluorophenyl)-6-(4-methylphenyl)-7H-[1,2,4]triazolo[3,4-b][1,3,4]thiadiazine;
3-(2,5-Dimethoxyphenyl)-6-(4-methylphenyl)-7H-[1,2,4]triazolo[3,4-b][1,3,4]thiadiazine;
3-(2-Methylphenyl)-6-(4-methylphenyl)-7H-[1,2,4]triazolo[3,4-b][1,3,4]thiadiazine;
3-(2-Methylthiophenyl)-6-(4-methylphenyl)-7H-[1,2,4]triazolo[3,4-b][1,3,4]thiadiazine;
3-(2-Methylsulfinylphenyl)-6-(4-methylphenyl)-7H-[1,2,4]triazolo[3,4-b][1,3,4]thiadiazine;
3-(2-Methylsulfonylphenyl)-6-(4-methylphenyl)-7H-[1,2,4]triazolo[3,4-b][1,3,4]thiadiazine;
3-(2-Aminophenyl)-6-(4-methylphenyl)-7H-[1,2,4]triazolo[3,4-b][1,3,4]thiadiazine;
6-(4-Aminophenyl)-3-(2-methoxyphenyl)-7H-[1,2,4]triazolo[3,4-b][1,3,4]thiadiazine;
6-(4-Azidophenyl)-3-(2-methoxyphenyl)-7H-[1,2,4]triazolo[3,4-b][1,3,4]thiadiazine;
6-(4-dimethylaminophenyl)-3-(2-methoxyphenyl)-7H-[1,2,4]triazolo[3,4-b][1,3,4]thiadiazine;
6-(4-Amino-3,5-dibromophenyl)-3-(2-methoxyphenyl)-7H-[1,2,4]triazolo[3,4-b][1,3,4]thiadiazine;
6-(4-Amino-3,5-diiodophenyl)-3-(2-methoxyphenyl)-7H-[1,2,4]triazolo[3,4-b][1,3,4]thiadiazine;
6-(4-Isopropylaminophenyl)-3-(2-methoxyphenyl)-7H-[1,2,4]triazolo[3,4-b][1,3,4]thiadiazine;
3-(4-Chloro-2-methylphenyl)-6-(4-methylphenyl)-7H-[1,2,4]triazolo[3,4-b][1,3,4]thiadiazine;
3-(2-Methoxyphenyl)-6-(3-methylphenyl)-7H-[1,2,4]triazolo[3,4-b][1,3,4]thiadiazine;
3-(2-Methylfuran-3-yl)-6-(4-methylphenyl)-7H-[1,2,4]triazolo[3,4-b][1,3,4]thiadiazine;
3-(2-Methylaminophenyl)-6-(4-methylphenyl)-7H-[1,2,4]triazolo[3,4-b][1,3,4]thiadiazine;
6-(4-Methoxycarbonylphenyl)-3-(2-methoxyphenyl)-7H-[1,2,4]triazolo[3,4-b][1,3,4]thiadiazine;
3-(2-Methoxyphenyl)-6-(1-methyl-1H-pyrrol-3-yl)-7H-[1,2,4]triazolo[3,4-b][1,3,4]thiadiazine;
6-(4-Cyanophenyl)-3-(2-methoxyphenyl)-7H-[1,2,4]triazolo[3,4-b][1,3,4]thiadiazine;
5,6-Dihydro-3-(2-methoxyphenyl)-6-(4-methylphenyl)-7H-[1,2,4]triazolo[3,4-b][1,3,4]thiadiazine;
6-(4-Carboxylphenyl)-3-(2-methoxyphenyl)-7H-[1,2,4]triazolo[3,4-b][1,3,4]thiadiazine;
3-(4-Chloro-2-methylphenyl)-6-(4-methyl-3-nitrophenyl)-7H-[1,2,4]triazolo[3,4-b][1,3,4]thiadiazine;
6-(3-Amino-4-methylphenyl)-3-(4-chloro-2-methylphenyl)-7H-[1,2,4]triazolo[3,4-b][1,3,4]thiadiazine;
6-(4-Methylphenyl)-3-(1H-pyrrol-2-yl)-7H-[1,2,4]triazolo[3,4-b][1,3,4]thiadiazine;
3-(4-Methyl-1,2,3-thiadiazol-5-yl)-6-(4-methylphenyl)-7H-[1,2,4]triazolo[3,4-b][1,3,4]thiadiazine;
3-(1-Methyl-1H-pyrrol-2-yl)-6-(4-methylphenyl)-7H-[1,2,4]triazolo[3,4-b][1,3,4]thiadiazine;
3-(1-Methyl-4-nitro-1H-pyrrol-2-yl)-6-(4-methylphenyl)-7H-[1,2,4]triazolo[3,4-b][1,3,4]thiadiazine;
6-(7-Bromoindolin-5-yl)-3-(2-methoxyphenyl)-7H-[1,2,4]triazolo[3,4-b][1,3,4]thiadiazine;
3-(4-Chloro-2-methoxyphenyl)-6-(4-methylphenyl)-7H-[1,2,4]triazolo[3,4-b][1,3,4]thiadiazine;
3-(1-Methyl-4-amino-1H-pyrrol-2-yl)-6-(4-methylphenyl)-7H-[1,2,4]triazolo[3,4-b][1,3,4]thiadiazine;
3-(2-Methyl-imidazo[1,2-a]pyridin-3-yl)-6-(4-methylphenyl)-7H-[1,2,4]triazolo[3,4-b][1,3,4]thiadiazine;

and pharmaceutically acceptable salts or prodrugs thereof.

The present invention is also directed to novel compounds within the scope of Formulae I-V. Exemplary preferred compounds that may be employed in this invention include, without limitation:
3-(3,5-dimethoxyphenyl)-6-(3,4-methylenedioxyphenyl)-7H-[1,2,4]triazolo[3,4-b][1,3,4]thiadiazine;
3-(3-Chloro-4,5-dimethoxyphenyl)-6-(3,4-methylenedioxyphenyl)-7H-[1,2,4]triazolo[3,4-b][1,3,4]thiadiazine;
3-(3-Chloro-4,5-dimethoxyphenyl)-6-(4-methoxyphenyl)-7H-[1,2,4]triazolo[3,4-b][1,3,4]thiadiazine;
3-(4-Chlorophenyl)-6-(4-hydroxyphenyl)-7H-[1,2,4]triazolo[3,4-b][1,3,4]thiadiazine;
6-(4-Hydroxyphenyl)-3-(2-methoxyphenyl)-7H-[1,2,4]triazolo[3,4-b][1,3,4]thiadiazine;
3-(2-Methoxyphenyl)-6-(4-methylphenyl)-7H-[1,2,4]triazolo[3,4-b][1,3,4]thiadiazine;
3-(2-Methoxyphenyl)-6-(4-(2-dimethylaminoethoxy)phenyl)-7H-[1,2,4]triazolo[3,4-b][1,3,4]thiadiazine;
6-(4-(2-Hydroxyethoxy)phenyl)-3-(2-methoxyphenyl)-7H-[1,2,4]triazolo[3,4-b][1,3,4]thiadiazine;
3-(2-Chlorophenyl)-6-(4-methylphenyl)-7H-[1,2,4]triazolo[3,4-b][1,3,4]thiadiazine;
3-(3,5-Dimethoxyphenyl)-6-(4-methylphenyl)-7H-[1,2,4]triazolo[3,4-b][1,3,4]thiadiazine;
3-(3,5-Dimethoxy-4-hydroxyphenyl)-6-(4-methylphenyl)-7H-[1,2,4]triazolo[3,4-b][1,3,4]thiadiazine;
3-(3,4-Dimethoxyphenyl)-6-(4-methylphenyl)-7H-[1,2,4]triazolo[3,4-b][1,3,4]thiadiazine;
3-(2,4-Dimethoxyphenyl)-6-(4-methylphenyl)-7H-[1,2,4]triazolo[3,4-b][1,3,4]thiadiazine;
3-(3,5-Dimethoxy-4-(2-dimethylaminoethoxy)phenyl)-6-(4-methylphenyl)-7H-[1,2,4]triazolo[3,4-b][1,3,4]thiadiazine;
3-(3,4,5-Trimethoxyphenyl)-6-(4-methylphenyl)-7H-[1,2,4]triazolo[3,4-b][1,3,4]thiadiazine;
3-(2,4-Dimethoxyphenyl)-6-(4-methoxyphenyl)-7H-[1,2,4]triazolo[3,4-b][1,3,4]thiadiazine;
3-(2-Ethoxyphenyl)-6-(4-methylphenyl)-7H-[1,2,4]triazolo[3,4-b][1,3,4]thiadiazine;
3-(3,5-Dimethoxyphenyl)-6-(4-methoxyphenyl)-7H-[1,2,4]triazolo[3,4-b][1,3,4]thiadiazine;
6-(4-Diethylaminophenyl)-3-(3,4,5-trimethoxyphenyl)-7H-[1,2,4]triazolo[3,4-b][1,3,4]thiadiazine;
6-(4-Diethylaminophenyl)-3-(3,4-dimethoxyphenyl)-7H-[1,2,4]triazolo[3,4-b][1,3,4]thiadiazine;
6-(4-Diethylaminophenyl)-3-(2-methoxyphenyl)-7H-[1,2,4]triazolo[3,4-b][1,3,4]thiadiazine;
3-(2-Methoxyphenyl)-6-(4-(pyrrolidin-1-yl)phenyl)-7H-[1,2,4]triazolo[3,4-b][1,3,4]thiadiazine;
3-(2-Methoxyphenyl)-6-(4-morpholinophenyl)-7H-[1,2,4]triazolo[3,4-b][1,3,4]thiadiazine;
6-(4-Diethylaminophenyl)-3-(3,5-dimethoxyphenyl)-7H-[1,2,4]triazolo[3,4-b][1,3,4]thiadiazine;
6-(4-Diethylaminophenyl)-3-(2,4-dimethoxyphenyl)-7H-[1,2,4]triazolo[3,4-b][1,3,4]thiadiazine;
3-(3,4-Dimethoxyphenyl)-6-(4-nitrophenyl)-7H-[1,2,4]triazolo[3,4-b][1,3,4]thiadiazine;
6-(4-Aminophenyl)-3-(3,4-dimethoxyphenyl)-7H-[1,2,4]triazolo[3,4-b][1,3,4]thiadiazine;
6-(4-Dimethylminophenyl)-3-(3,4-dimethoxyphenyl)-7H-[1,2,4]triazolo[3,4-b][1,3,4]thiadiazine;
6-(4-Azidophenyl)-3-(3,4-dimethoxyphenyl)-7H-[1,2,4]triazolo[3,4-b][1,3,4]thiadiazine;
6-(4-Acetamidophenyl)-3-(3,4-dimethoxyphenyl)-7H-[1,2,4]triazolo[3,4-b][1,3,4]thiadiazine;
6-(2,3-Dihydro-2-oxobenzo[d]oxazol-6-yl)-3-(2,4-Dimethoxyphenyl)-7H-[1,2,4]triazolo[3,4-b][1,3,4]thiadiazine;
6-(2,3-Dihydro-2-oxobenzo[d]oxazol-6-yl)-3-(3,5-dimethoxyphenyl)-7H-[1,2,4]triazolo[3,4-b][1,3,4]thiadiazine;
6-(2,3-Dihydro-2-oxobenzo[d]oxazol-6-yl)-3-(3,4-dimethoxyphenyl)-7H-[1,2,4]triazolo[3,4-b][1,3,4]thiadiazine;
3-(2-Methoxyphenyl)-6-(4-methoxyphenyl)-[1,2,4]triazolo[4,3-b][1,2,4]triazin-7(8H)-one;
3-(3,4,5-Trimethoxyphenyl)-6-(4-methylphenyl)-1H-[1,2,4]triazolo[4,3-b][1,2,4]triazin-7-one;
3-(2-Methoxyphenyl)-6-(4-methylphenyl)-1H-[1,2,4]triazolo[4,3-b][1,2,4]triazin-7-one;
3-(2-Methoxyphenyl)-6-(2,4-dimethylphenyl)-7H-[1,2,4]triazolo[3,4-b][1,3,4]thiadiazine;
3-(2-Methoxyphenyl)-6-(3,4-dimethylphenyl)-7H-[1,2,4]triazolo[3,4-b][1,3,4]thiadiazine;
6-(2-Methoxy-4-methylphenyl)-3-(2-methoxyphenyl)-7H-[1,2,4]triazolo[3,4-b][1,3,4]thiadiazine;
6-(2,3,4-Trimethoxyphenyl)-3-(2-methoxyphenyl)-7H-[1,2,4]triazolo[3,4-b][1,3,4]thiadiazine;
6-(2,3-Dihydrobenzo[b][1,4]dioxin-6-yl)-3-(2-methoxyphenyl)-7H-[1,2,4]triazolo[3,4-b][1,3,4]thiadiazine;
6-(4-Isopropylphenyl)-3-(2-methoxyphenyl)-7H-[1,2,4]triazolo[3,4-b][1,3,4]thiadiazine;
6-(3-Methoxy-4-nitrophenyl)-3-(2-methoxyphenyl)-7H-[1,2,4]triazolo[3,4-b][1,3,4]thiadiazine;
3-(2-Methoxyphenyl)-6-(4-methoxyphenyl)-7H-[1,2,4]triazolo[3,4-b]-[1,3,4]thiadiazine;
6-(2,3-Dihydro-2-oxobenzo[d]oxazol-6-yl)-3-(2-methoxyphenyl)-7H-[1,2,4]triazolo[3,4-b][1,3,4]thiadiazine;
6-(4-(Trifluoromethyl)phenyl)-3-(2-methoxyphenyl)-7H-[1,2,4]triazolo[3,4-b][1,3,4]thiadiazine;
6-(5-Chloro-2-methoxy-4-methylphenyl)-3-(2-methoxyphenyl)-7H-[1,2,4]triazolo[3,4-b][1,3,4]thiadiazine;
3-(2-Methoxyphenyl)-6-(4-(trifluoromethoxy)phenyl)-7H-[1,2,4]triazolo[3,4-b][1,3,4]thiadiazine;
6-(4-tert-Butylphenyl)-3-(2-methylphenyl)-7H-[1,2,4]triazolo[3,4-b][1,3,4]thiadiazine;
6-(4-(N,N-Diethylaminosulfonyl)phenyl)-3-(2-methylphenyl)-7H-[1,2,4]triazolo[3,4-b][1,3,4]thiadiazine;
6-(4-Methyl-3-nitrophenyl)-3-(2-methylphenyl)-7H-[1,2,4]triazolo[3,4-b][1,3,4]thiadiazine;
3-(2-Methoxyphenyl)-6-(4-methyl-3-nitrophenyl)-7H-[1,2,4]triazolo[3,4-b][1,3,4]thiadiazine;
6-(4-Chloro-3-nitrophenyl)-3-(2-methoxyphenyl)-7H-[1,2,4]triazolo[3,4-b][1,3,4]thiadiazine;
6-(4-Acetamido-3-nitrophenyl)-3-(2-methoxyphenyl)-7H-[1,2,4]triazolo[3,4-b][1,3,4]thiadiazine;
6-(4-Amino-3-methoxyphenyl)-3-(2-methoxyphenyl)-7H-[1,2,4]triazolo[3,4-b][1,3,4]thiadiazine;
6-(3-Amino-4-methylphenyl)-3-(2-methoxyphenyl)-7H-[1,2,4]triazolo[3,4-b][1,3,4]thiadiazin;
6-(3-Amino-4-chlorophenyl)-3-(2-methoxyphenyl)-7H-[1,2,4]triazolo[3,4-b][1,3,4]thiadiazine;
6-(3,4-Diaminophenyl)-3-(2-methoxyphenyl)-7H-[1,2,4]triazolo[3,4-b][1,3,4]thiadiazine;
6-(3-Amino-4-methylphenyl)-3-(2-methylphenyl)-7H-[1,2,4]triazolo[3,4-b][1,3,4]thiadiazine;
3-(5-Chloro-2-methoxyphenyl)-6-(4-methylphenyl)-7H-[1,2,4]triazolo[3,4-b][1,3,4]thiadiazine;
3-(2-Methoxyphenyl)-6-(3-methoxylphenyl)-7H-[1,2,4]triazolo[3,4-b][1,3,4]thiadiazine;
3-(2-Methoxyphenyl)-6-(2-methoxylphenyl)-7H-[1,2,4]triazolo[3,4-b][1,3,4]thiadiazine;
3-(2,3-Dimethoxyphenyl)-6-(4-methylphenyl)-7H-[1,2,4]triazolo[3,4-b][1,3,4]thiadiazine;
6-(4-Aminophenyl)-3-(3,4,5-trimethoxyphenyl)-7H-[1,2,4]triazolo[3,4-b][1,3,4]thiadiazine;
6-(4-Dimethylaminophenyl)-3-(3,4,5-trimethoxyphenyl)-7H-[1,2,4]triazolo[3,4-b][1,3,4]thiadiazine;
3-(2-Fluorophenyl)-6-(4-methylphenyl)-7H-[1,2,4]triazolo[3,4-b][1,3,4]thiadiazine;
3-(2,5-Dimethoxyphenyl)-6-(4-methylphenyl)-7H-[1,2,4]triazolo[3,4-b][1,3,4]thiadiazine;
3-(2-Methylphenyl)-6-(4-methylphenyl)-7H-[1,2,4]triazolo[3,4-b][1,3,4]thiadiazine;
3-(2-Methylthiophenyl)-6-(4-methylphenyl)-7H-[1,2,4]triazolo[3,4-b][1,3,4]thiadiazine;
3-(2-Methylsulfinylphenyl)-6-(4-methylphenyl)-7H-[1,2,4]triazolo[3,4-b][1,3,4]thiadiazine;
3-(2-Methylsulfonylphenyl)-6-(4-methylphenyl)-7H-[1,2,4]triazolo[3,4-b][1,3,4]thiadiazine;
3-(2-Aminophenyl)-6-(4-methylphenyl)-7H-[1,2,4]triazolo[3,4-b][1,3,4]thiadiazine;
6-(4-Aminophenyl)-3-(2-methoxyphenyl)-7H-[1,2,4]triazolo[3,4-b][1,3,4]thiadiazine;
6-(4-Azidophenyl)-3-(2-methoxyphenyl)-7H-[1,2,4]triazolo[3,4-b][1,3,4]thiadiazine;
6-(4-dimethylaminophenyl)-3-(2-methoxyphenyl)-7H-[1,2,4]triazolo[3,4-b][1,3,4]thiadiazine;
6-(4-Amino-3,5-dibromophenyl)-3-(2-methoxyphenyl)-7H-[1,2,4]triazolo[3,4-b][1,3,4]thiadiazine;
6-(4-Amino-3,5-diiodophenyl)-3-(2-methoxyphenyl)-7H-[1,2,4]triazolo[3,4-b][1,3,4]thiadiazine;
6-(4-Isopropylaminophenyl)-3-(2-methoxyphenyl)-7H-[1,2,4]triazolo[3,4-b][1,3,4]thiadiazine;
3-(4-Chloro-2-methylphenyl)-6-(4-methylphenyl)-7H-[1,2,4]triazolo[3,4-b][1,3,4]thiadiazine;
3-(2-Methoxyphenyl)-6-(3-methylphenyl)-7H-[1,2,4]triazolo[3,4-b][1,3,4]thiadiazine;
3-(2-Methylfuran-3-yl)-6-(4-methylphenyl)-7H-[1,2,4]triazolo[3,4-b][1,3,4]thiadiazine;
3-(2-Methylaminophenyl)-6-(4-methylphenyl)-7H-[1,2,4]triazolo[3,4-b][1,3,4]thiadiazine;
6-(4-Methoxycarbonylphenyl)-3-(2-methoxyphenyl)-7H-[1,2,4]triazolo[3,4-b][1,3,4]thiadiazine;
3-(2-Methoxyphenyl)-6-(1-methyl-1H-pyrrol-3-yl)-7H-[1,2,4]triazolo[3,4-b][1,3,4]thiadiazine;
6-(4-Cyanophenyl)-3-(2-methoxyphenyl)-7H-[1,2,4]triazolo[3,4-b][1,3,4]thiadiazine;
5,6-Dihydro-3-(2-methoxyphenyl)-6-(4-methylphenyl)-7H-[1,2,4]triazolo[3,4-b][1,3,4]thiadiazine;
6-(4-Carboxylphenyl)-3-(2-methoxyphenyl)-7H-[1,2,4]triazolo[3,4-b][1,3,4]thiadiazine;
3-(4-Chloro-2-methylphenyl)-6-(4-methyl-3-nitrophenyl)-7H-[1,2,4]triazolo[3,4-b][1,3,4]thiadiazine;
6-(3-Amino-4-methylphenyl)-3-(4-chloro-2-methylphenyl)-7H-[1,2,4]triazolo[3,4-b][1,3,4]thiadiazine;
6-(4-Methylphenyl)-3-(1H-pyrrol-2-yl)-7H-[1,2,4]triazolo[3,4-b][1,3,4]thiadiazine;
3-(4-Methyl-1,2,3-thiadiazol-5-yl)-6-(4-methylphenyl)-7H-[1,2,4]triazolo[3,4-b][1,3,4]thiadiazine;
3-(1-Methyl-1H-pyrrol-2-yl)-6-(4-methylphenyl)-7H-[1,2,4]triazolo[3,4-b][1,3,4]thiadiazine;
3-(1-Methyl-4-nitro-1H-pyrrol-2-yl)-6-(4-methylphenyl)-7H-[1,2,4]triazolo[3,4-b][1,3,4]thiadiazine;
6-(7-Bromoindolin-5-yl)-3-(2-methoxyphenyl)-7H-[1,2,4]triazolo[3,4-b][1,3,4]thiadiazine;
3-(4-Chloro-2-methoxyphenyl)-6-(4-methylphenyl)-7H-[1,2,4]triazolo[3,4-b][1,3,4]thiadiazine;
3-(1-Methyl-4-amino-1H-pyrrol-2-yl)-6-(4-methylphenyl)-7H-[1,2,4]triazolo[3,4-b][1,3,4]thiadiazine;
3-(2-Methyl-imidazo[1,2-a]pyridin-3-yl)-6-(4-methylphenyl)-7H-[1,2,4]triazolo[3,4-b][1,3,4]thiadiazine;

and pharmaceutically acceptable salts or prodrugs thereof.

The term “alkyl” as employed herein by itself or as part of another group refers to both straight and branched chain radicals of up to ten carbons. Useful alkyl groups include straight-chained and branched C₁₋₁₀ alkyl groups, more preferably C₁₋₆ alkyl groups. Typical C₁₋₁₀ alkyl groups include methyl, ethyl, propyl, isopropyl, butyl, sec-butyl, tert-butyl, 3-pentyl, hexyl and octyl groups, which may be optionally substituted.

The term “amino” as employed herein by itself or as part of another group is —NH₂, —NHRₐ, or —NRₐR_{b}, wherein Rₐ and R_{b} are independently alkyl groups or together, with the nitrogen, form a 5 or 6 membered heterocyclo group optionally containing an additional N or O atom.

The term “alkenyl” as employed herein by itself or as part of another group means a straight or branched chain radical of 2-10 carbon atoms, unless the chain length is limited thereto, including at least one double bond between two of the carbon atoms in the chain. Typical alkenyl groups include ethenyl, 1-propenyl, 2-propenyl, 2-methyl-1-propenyl, 1-butenyl and 2-butenyl.

The term “alkynyl” is used herein to mean a straight or branched chain radical of 2-10 carbon atoms, unless the chain length is limited thereto, wherein there is at least one triple bond between two of the carbon atoms in the chain. Typical alkynyl groups include ethynyl, 1-propynyl, 1-methyl-2-propynyl, 2-propynyl, 1-butynyl and 2-butynyl.

Useful alkoxy groups include oxygen substituted by one of the C₁₋₁₀ alkyl groups mentioned above, which may be optionally substituted. Alkoxy substituents include, without limitation, halo, morpholino, amino including alkylamino and dialkylamino, and carboxy including esters thereof.

Useful alkylthio groups include sulfur substituted by one of the C₁₋₁₀ alkyl groups mentioned above, which may be optionally substituted. Also included are the sulfoxides and sulfones of such alkylthio groups.

Useful amino and optionally substituted amino groups include —NH₂, —NHR₁₅ and —NR₁₅R₁₆, wherein R₁₅ and R₁₆ are C₁₋₁₀ alkyl or cycloalkyl groups, or R₁₅ and R₁₆ are combined with the N to form a ring structure, such as a piperidine, or R₁₅ and R₁₆ are combined with the N and other group to form a ring, such as a piperazine. The alkyl group may be optionally substituted.

Optional substituents on the alkyl, alkoxy, alkylthio, alkenyl, alkynyl, cycloalkyl, carbocyclic and heterocyclic groups include one or more halo, hydroxy, carboxyl, amino, nitro, cyano, C₁-C₆ acylamino, C₁-C₆ acyloxy, C₁-C₆ alkoxy, aryloxy, alkylthio, C₆-C₁₀ aryl, C₄-C₇ cycloalkyl, C₂-C₆ alkenyl, C₂-C₆ alkynyl, C₆-C₁₀ aryl(C₂-C₆)alkenyl, C₆-C₁₀ aryl(C₂-C₆)alkynyl, saturated and unsaturated heterocyclic or heteroaryl.

Optional substituents on the aryl, arylalkyl, arylalkenyl, arylalkynyl and heteroaryl and heteroarylalkyl groups include one or more halo, methylenedioxy, C₁-C₆ haloalkyl, C₆-C₁₀ aryl, C₄-C₇ cycloalkyl, C₁-C₆ alkyl, C₂-C₆ alkenyl, C₂-C₆ alkynyl, C₆-C₁₀ aryl(C₁-C₆)alkyl, C₆-C₁₀ aryl(C₂-C₆)alkenyl, C₆-C₁₀ aryl(C₂-C₆)alkynyl, C₁-C₆ hydroxyalkyl, nitro, amino, ureido, cyano, C₁-C₆ acylamino, hydroxy, thiol, C₁-C₆ acyloxy, azido, C₁-C₆ alkoxy, carboxy, di(C₁₋₁₀ alkyl)amino, alkylsulfonyl, aminosulfonyl, dialkylaminosulfonyl, or alkylsulfiniyl.

The term “aryl” as employed herein by itself or as part of another group refers to monocyclic, bicyclic or tricyclic aromatic groups containing from 6 to 14 carbons in the ring portion.

Useful aryl groups include C₆₋₁₄ aryl, preferably C₆₋₁₀ aryl. Typical C₆₋₁₄ aryl groups include phenyl, naphthyl, phenanthrenyl, anthracenyl, indenyl, azulenyl, biphenyl, biphenylenyl and fluorenyl groups.

The term “carbocycle” as employed herein include cycloalkyl and partially saturated carbocyclic groups. Useful cycloalkyl groups are C₃₋₈ cycloalkyl. Typical cycloalkyl groups include cyclopropyl, cyclobutyl, cyclopentyl, cyclohexyl and cycloheptyl.

Useful saturated or partially saturated carbocyclic groups are cycloalkyl groups as described above, as well as cycloalkenyl groups, such as cyclopentenyl, cycloheptenyl and cyclooctenyl.

Useful halo or halogen groups include fluorine, chlorine, bromine and iodine.

The term “arylalkyl” is used herein to mean any of the above-mentioned C₁₋₁₀ alkyl groups substituted by any of the above-mentioned C₆₋₁₄ aryl groups. Preferably the arylalkyl group is benzyl, phenethyl or naphthylmethyl.

The term “arylalkenyl” is used herein to mean any of the above-mentioned C₂₋₁₀ alkenyl groups substituted by any of the above-mentioned C₆₋₁₄ aryl groups.

The term “arylalkynyl” is used herein to mean any of the above-mentioned C₂₋₁₀ alkynyl groups substituted by any of the above-mentioned C₆₋₁₄ aryl groups.

The term “aryloxy” is used herein to mean oxygen substituted by one of the above-mentioned C₆₋₁₄ aryl groups, which may be optionally substituted. Useful aryloxy groups include phenoxy and 4-methylphenoxy.

The term “arylalkoxy” is used herein to mean any of the above mentioned C₁₋₁₀ alkoxy groups substituted by any of the above-mentioned aryl groups, which may be optionally substituted. Useful arylalkoxy groups include benzyloxy and phenethyloxy.

Useful haloalkyl groups include C₁₋₁₀ alkyl groups substituted by one or more fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

Useful acylamino (acylamido) groups are any C₁₋₆ acyl (alkanoyl) attached to an amino nitrogen, e.g., acetamido, chloroacetamido, propionamido, butanoylamido, pentanoylamido and hexanoylamido, as well as aryl-substituted C₁₋₆ acylamino groups, e.g., benzoylamido, and pentafluorobenzoylamido.

Useful acyloxy groups are any C₁₋₆ acyl (alkanoyl) attached to an oxy (—O—) group, e.g., formyloxy, acetoxy, propionoyloxy, butanoyloxy, pentanoyloxy and hexanoyloxy.

The term heterocycle is used herein to mean a saturated or partially saturated 3-7 membered monocyclic, or 7-10 membered bicyclic ring system, which consists of carbon atoms and from one to four heteroatoms independently selected from the group consisting of O, N, and S, wherein the nitrogen and sulfur heteroatoms can be optionally oxidized, the nitrogen can be optionally quaternized, and including any bicyclic group in which any of the above-defined heterocyclic rings is fused to a benzene ring, and wherein the heterocyclic ring can be substituted on carbon or on a nitrogen atom if the resulting compound is stable.

Useful saturated or partially saturated heterocyclic groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl pyrazolinyl, tetronoyl and tetramoyl groups.

The term “heteroaryl” as employed herein refers to groups having 5 to 14 ring atoms; 6, 10 or 14 π electrons shared in a cyclic array; and containing carbon atoms and 1, 2 or 3 oxygen, nitrogen or sulfur heteroatoms.

Useful heteroaryl groups include thienyl (thiophenyl), benzo[b]thienyl, naphtho[2,3-b]thienyl, thianthrenyl, furyl (furanyl), pyranyl, isobenzofuranyl, chromenyl, xanthenyl, phenoxanthiinyl, pyrrolyl, including without limitation pyrrolyl, including 1H-pyrrolyl, 2H-pyrrolyl, and 3H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-amino-isocoumarin, pyrido[1,2-a]pyrimidin-4-one, tetrahydrocyclopenta[c]pyrazol-3-yl, pyrazolo[1,5-a]pyrimidinyl, including without limitation pyrazolo[1,5-a]pyrimidin-3-yl, 1,2-benzoisoxazol-3-yl, benzimidazolyl, 2-oxindolyl, thiadiazolyl, including 1,2,3-thiadiazolyl, 1,3,4-thiadiazolyl, 1,2,4-thiadiazolyl, and 1,2,5-thiadiazolyl, and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

The term “heteroaryloxy” is used herein to mean oxygen substituted by one of the above-mentioned heteroaryl groups, which may be optionally substituted. Useful heteroaryloxy groups include pyridyloxy, pyrazinyloxy, pyrrolyloxy, pyrazolyloxy, imidazolyloxy and thiophenyloxy.

The term “heteroarylalkoxy” is used herein to mean any of the above-mentioned C₁₋₁₀ alkoxy groups substituted by any of the above-mentioned heteroaryl groups, which may be optionally substituted.

Some of the compounds of the present invention may exist as stereoisomers including optical isomers. The invention includes all stereoisomers and both the racemic mixtures of such stereoisomers as well as the individual enantiomers that may be separated according to methods that are well known to those of ordinary skill in the art.

Examples of pharmaceutically acceptable addition salts include inorganic and organic acid addition salts, such as hydrochloride, hydrobromide, phosphate, sulphate, citrate, lactate, tartrate, maleate, fumarate, mandelate and oxalate; and inorganic and organic base addition salts with bases, such as sodium hydroxy, Tris(hydroxymethyl)aminomethane (TRIS, tromethane) and N-methyl-glucamine.

Examples of prodrugs of the compounds of the invention include the simple esters of carboxylic acid containing compounds (e.g., those obtained by condensation with a C₁₋₄ alcohol according to methods known in the art); esters of hydroxy containing compounds (e.g., those obtained by condensation with a C₁₋₄ carboxylic acid, C₃₋₆ dioic acid or anhydride thereof, such as succinic and fumaric anhydrides according to methods known in the art); imines of amino containing compounds (e.g., those obtained by condensation with a C₁₋₄ aldehyde or ketone according to methods known in the art); carbamate of amino containing compounds, such as those described by Leu, et. al., (J. Med. Chem. 42:3623-3628 (1999)) and Greenwald, et. al., (J. Med. Chem. 42:3657-3667 (1999)); and acetals and ketals of alcohol containing compounds (e.g., those obtained by condensation with chloromethyl methyl ether or chloromethyl ethyl ether according to methods known in the art).

The compounds of this invention may be prepared using methods known to those skilled in the art, or the novel methods of this invention. Specifically, the compounds of this invention with Formulae I, II and V can be prepared as illustrated by the exemplary reaction in Scheme 1. Reaction of a substituted benzoic acid, such as 3,5-dimethoxybenzoic acid, and thiocarbohydrazide produced 4-amino-5-(3,5-dimethoxyphenyl)-3-mercapto-(4H)-1,2,4-triazole. Reaction of 4-amino-5-(3,5-dimethoxyphenyl)-3-mercapto-(4H)-1,2,4-triazole with a substituted 1-aryl-2-bromoethanone, such as 2-bromo-1-(3,4-methylenedioxyphenyl)ethanone, in a solvent such as ethanol produced 3-(3,5-dimethoxyphenyl)-6-(3,4-methylenedioxyphenyl)-7H-[1,2,4]triazolo[3,4-b][1,3,4]thiadiazine.

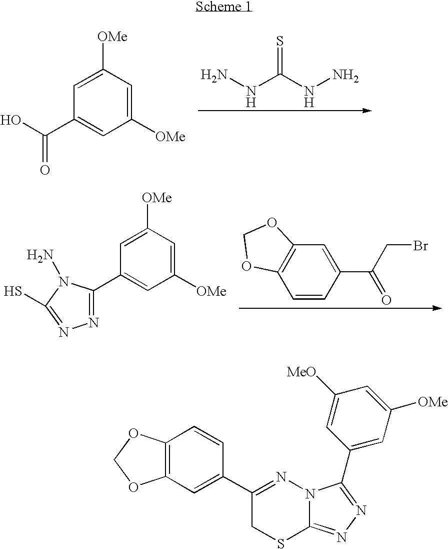

Other compounds of this invention may be prepared similarly as illustrated by the exemplary reaction in Scheme 2. Reaction of 4-chloro-2-methylbenzohydrazide with carbon disulfide in ethanol in the presence of a base such as KOH produced 4-chloro-2-methylbenzoyl-2-dithiocarboxyhydrazide as a potassium salt. Reaction of the potassium salt of 4-chloro-2-methylbenzoyl-2-dithiocarboxyhydrazide with hydrazine hydrate in ethanol and water produced 4-amino-5-(4-chloro-2-methylphenyl)-3-mercapto-(4H)-1,2,4-triazole. Reaction of 4-amino-5-(4-chloro-2-methylphenyl)-3-mercapto-(4H)-1,2,4-triazole with 2-bromo-1-(4-methylphenyl)ethanone in isopropanol produced 3-(4-chloro-2-methylphenyl)-6-(4-methylphenyl)-7H-[1,2,4]triazolo[3,4-b][1,3,4]thiadiazine.

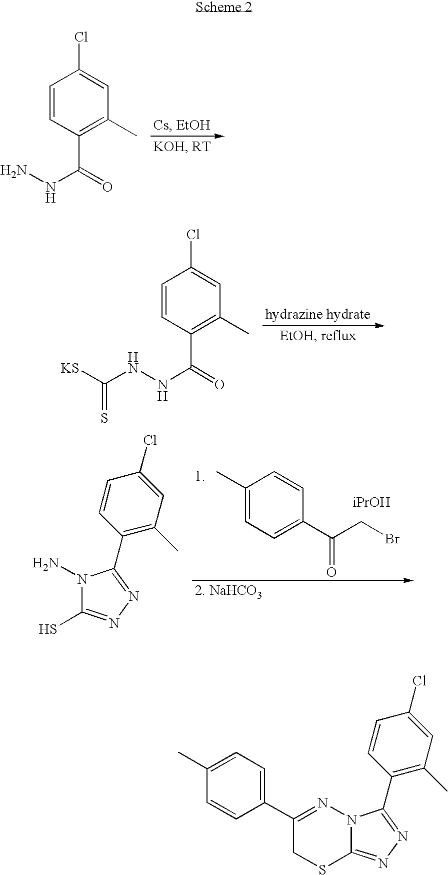

Other compounds of this invention may be prepared similarly as illustrated by the exemplary reaction in Scheme 3. Reaction of 4-amino-5-(2-methoxyphenyl)-3-mercapto-(4H)-1,2,4-triazole with 2-bromo-1-(4-methyl-3-nitrophenyl)ethanone in isopropanol produced 3-(2-methoxyphenyl)-6-(4-methyl-3-nitrophenyl)-7H-[1,2,4]triazolo[3,4-b][1,3,4]thiadiazine. The nitro group was reduced by treatment with tin (II) chloride dihydrate to produce the amino compound 6-(3-amino-4-methylphenyl)-3-(2-methoxyphenyl)-7H-[1,2,4]triazolo[3,4-b][1,3,4]thiadiazine. The HCl salt was prepared via treatment with HCl in ether/methanol.

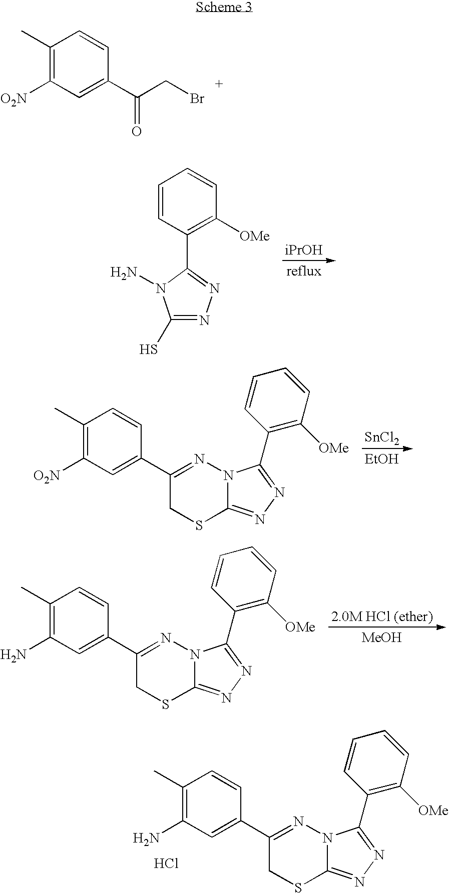

Other compounds of this invention may be prepared similarly as illustrated by the exemplary reaction in Scheme 4. Reaction of 3-(2-methoxyphenyl)-6-(4-methylphenyl)-7H-[1,2,4]triazolo[3,4-b][1,3,4]thiadiazine with sodium borohydride in dioxane and water produced 5,6-dihydro-3-(2-methoxyphenyl)-6-(4-methylphenyl)-7H-[1,2,4]triazolo[3,4-b][1,3,4]thiadiazines with the C═N double bond in the center ring reduced.

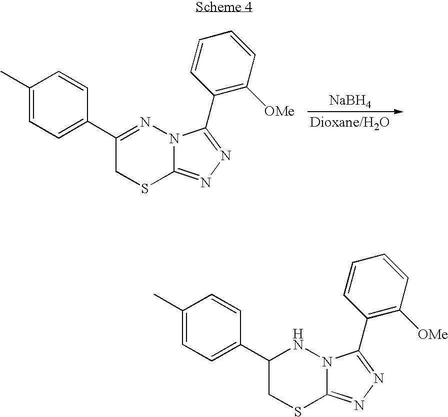

Compounds of this invention also may be prepared as illustrated by the exemplary reaction in Scheme 5. Reaction of 6-(2-chloroacetyl)benzo[d]oxazol-2(3H)-one and 4-amino-5-(2,4-dimethoxyphenyl)-3-mercapto-(4H)-1,2,4-triazole in anhydrous isopropanol produced the intermediate 6-(2-(5-(2,4-dimethoxyphenyl)-4-amino-4H-1,2,4-triazol-3-ylthio)acetyl)benzo[d]oxazol-2(3H)-one. Treatment of 6-(2-(5-(2,4-dimethoxyphenyl)-4-amino-4H-1,2,4-triazol-3-ylthio)acetyl)benzo[d]oxazol-2(3H)-one in anhydrous toluene with catalytic amounts of p-toluenesulfonic acid in a Dean-Stark apparatus produced 6-(2,3-dihydro-2-oxobenzo[d]oxazol-6-yl)-3-(2,4-dimethoxyphenyl)-7H-[1,2,4]triazolo[3,4-b][1,3,4]thiadiazine.

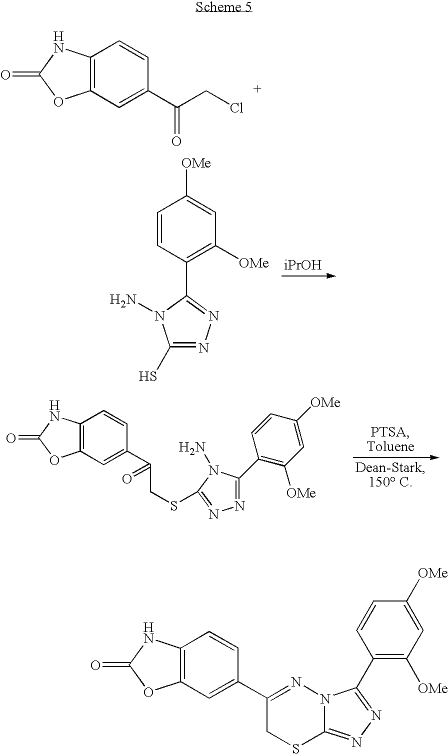

Compounds of this invention with Formulae I may be prepared as illustrated by the exemplary reaction in Scheme 6. Reaction of 3,4-dihydro-6-(4-methoxyphenyl)-3-thioxo-1,2,4-triazin-5(2H)-one (2.00 g, 8.50 mmol) with iodomethane in 1N NaOH produced 6-(4-methoxyphenyl)-3-(methylthio)-1,2,4-triazin-5(2H)-one, which was treated with hydrazine in ethanol to produce 3-hydrazinyl-6-(4-methoxyphenyl)-1,2,4-triazin-5(2H)-one. Reaction of 3-hydrazinyl-6-(4-methoxyphenyl)-1,2,4-triazin-5(2H)-one with 2-methoxybenzoyl chloride in anhydrous pyridine, and produced 3-(2-methoxyphenyl)-6-(4-methoxyphenyl)-[1,2,4]triazolo[4,3-b][1,2,4]triazin-7(8H)-one.

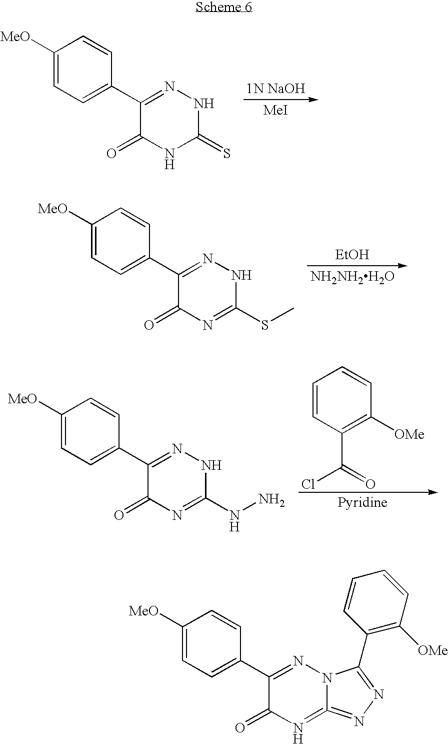

Compounds of this invention with Formulae I and III may be prepared as illustrated by the exemplary reaction in Scheme 7. Reaction of a substituted benzohydrazide, such as 3,4,5-trimethoxybenzohydrazide and phosgene will produce 5-(3,4,5-trimethoxyphenyl)-1,3,4-oxadiazol-2(3H)-one. Reaction of 5-(3,4,5-trimethoxyphenyl)-1,3,4-oxadiazol-2(3H)-one with hydrazine will produce 4-amino-5-(3,4,5-trimethoxyphenyl)-3-hydroxy-(4H)-1,2,4-triazole. Reaction of 4-amino-5-(3,4,5-trimethoxyphenyl)-3-hydroxy-(4H)-1,2,4-triazole with a substituted 1-aryl-2-bromoethanone, such as 2-bromo-1-(3,4-methylenedioxyphenyl)ethanone, will produce 3-(3,4,5-trimethoxyphenyl)-6-(3,4-methylenedioxyphenyl)-7H-[1,2,4]triazolo[3,4-b][1,3,4]oxadiazine.

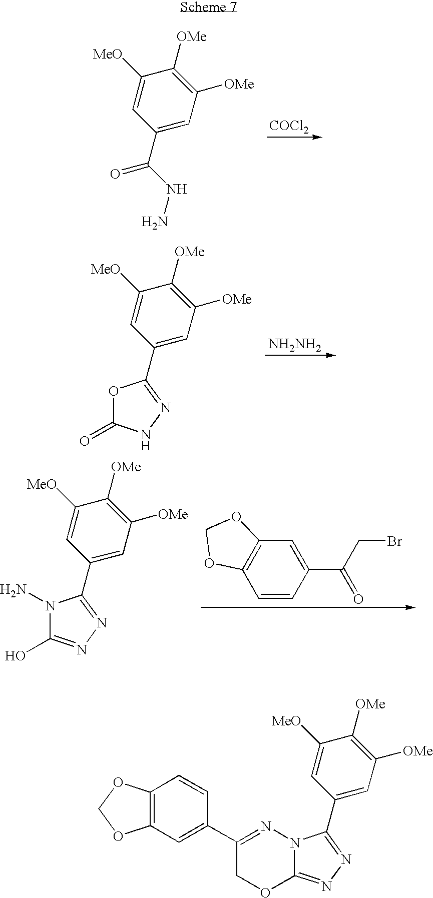

Compounds of this invention with Formulae I and IV may be prepared as illustrated by the exemplary reaction in Scheme 8. Reaction of a substituted benzoic acid, such as 3,4,5-trimethoxybenzoic acid and thiosemicarbazide will produce 5-(3,4,5-trimethoxyphenyl)-1,3,4-thiadiazol-2-amine. Reaction of 5-(3,4,5-trimethoxyphenyl)-1,3,4-thiadiazol-2-amine with hydrazine will produce 3,4-diamino-5-(3,4,5-trimethoxyphenyl)-1,2,4-triazole. Reaction of 3,4-diamino-5-(3,4,5-trimethoxyphenyl)-1,2,4-triazole with a substituted 1-aryl-2-bromoethanone, such as 2-bromo-1-(3,4-methylenedioxyphenyl)ethanone, will produce 6-(3,4-methylenedioxyphenyl)-7,8-dihydro-3-(3,4,5-trimethoxyphenyl)-[1,2,4]triazolo[4,3-b][1,2,4]triazine.

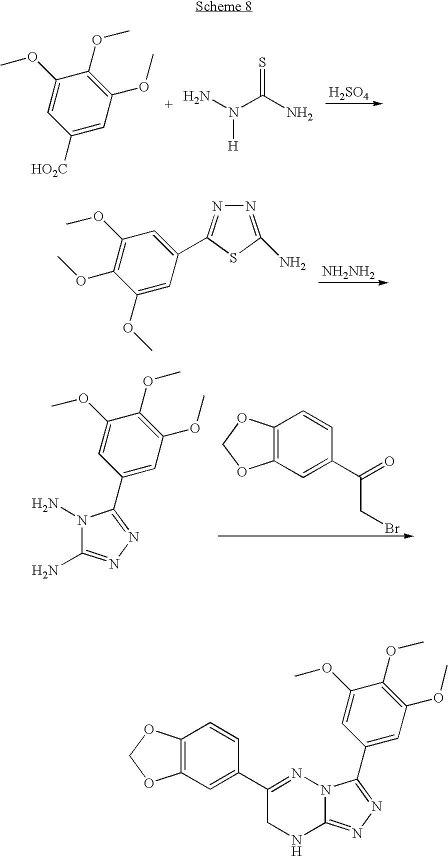

Compounds of this invention with Formulae I may be prepared as illustrated by the exemplary reaction in Scheme 9. Reaction of 3,4-diamino-5-(3,4,5-trimethoxyphenyl)-1,2,4-triazole with a substituted 2-aryl-2-oxoacetic acid, such as 2-(4-methoxyphenyl)-2-oxoacetic acid, will produce 3-(3,4,5-trimethoxyphenyl)-6-(4-methoxyphenyl)-[1,2,4]triazolo[4,3-b][1,2,4]triazin-7(8H)-one.

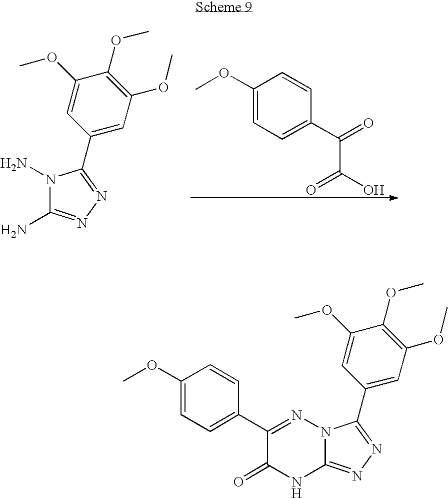

An important aspect of the present invention is the discovery that compounds having Formulae I-V are activators of caspases and inducers of apoptosis. Therefore, these compounds are useful in a variety of clinical conditions in which there is uncontrolled cell growth and spread of abnormal cells, such as in the case of cancer.

Another important aspect of the present invention is the discovery that compounds having Formulae I-V are potent and highly efficacious activators of caspases and inducers of apoptosis in drug resistant cancer cells, such as breast and prostate cancer cells, which enables these compounds to kill these drug resistant cancer cells. In comparison, most standard anti-cancer drugs are not effective in killing drug resistant cancer cells under the same conditions. Therefore, compounds of this invention are useful for the treatment of drug resistant cancer, such as breast cancer in animals.

The present invention includes a therapeutic method useful to modulate in vivo apoptosis or in vivo neoplastic disease, comprising administering to a subject in need of such treatment an effective amount of a compound, or a pharmaceutically acceptable salt or prodrug of the compound of Formulae I-V, which functions as a caspase cascade activator and inducer of apoptosis.

The present invention also includes a therapeutic method comprising administering to an animal an effective amount of a compound, or a pharmaceutically acceptable salt or prodrug of said compound of Formulae I-V, wherein said therapeutic method is useful to treat cancer, which is a group of diseases characterized by the uncontrolled growth and spread of abnormal cells. Such diseases include, but are not limited to, Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

In practicing the therapeutic methods, effective amounts of compositions containing therapeutically effective concentrations of the compounds formulated for oral, intravenous, local and topical application, for the treatment of neoplastic diseases and other diseases in which caspase cascade mediated physiological responses are implicated, are administered to an individual exhibiting the symptoms of one or more of these disorders. The amounts are effective to ameliorate or eliminate one or more symptoms of the disorders. An effective amount of a compound for treating a particular disease is an amount that is sufficient to ameliorate, or in some manner reduce, the symptoms associated with the disease. Such amount may be administered as a single dosage or may be administered according to a regimen, whereby it is effective. The amount may cure the disease but, typically, is administered in order to ameliorate the symptoms of the disease. Typically, repeated administration is required to achieve the desired amelioration of symptoms.

In another embodiment, a pharmaceutical composition comprising a compound, or a pharmaceutically acceptable salt of said compound of Formulae I-V, which functions as a caspase cascade activator and inducer of apoptosis in combination with a pharmaceutically acceptable vehicle is provided.

Another embodiment of the present invention is directed to a composition effective to inhibit neoplasia comprising a compound, or a pharmaceutically acceptable salt or prodrug of said compound of Formulae I-V, which functions as a caspase cascade activator and inducer of apoptosis, in combination with at least one known cancer chemotherapeutic agent, or a pharmaceutically acceptable salt of said agent. Examples of known cancer chemotherapeutic agents which may be used for combination therapy include, but not are limited to alkylating agents, such as busulfan, cis-platin, mitomycin C, and carboplatin; antimitotic agents, such as colchicine, vinblastine, paclitaxel, and docetaxel; topo I inhibitors, such as camptothecin and topotecan; topo II inhibitors, such as doxorubicin and etoposide; RNA/DNA antimetabolites, such as 5-azacytidine, 5-fluorouracil and methotrexate; DNA antimetabolites, such as 5-fluoro-2′-deoxy-uridine, ara-C, hydroxyurea and thioguanine; antibodies, such as campath, Herceptin® or Rituxan®. Other known cancer chemotherapeutic agents which may be used for combination therapy include melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen, Gleevec® and alanosine.

In practicing the methods of the present invention, the compound of the invention may be administered together with at least one known chemotherapeutic agent as part of a unitary pharmaceutical composition. Alternatively, the compound of the invention may be administered apart from at least one known cancer chemotherapeutic agent. In one embodiment, the compound of the invention and at least one known cancer chemotherapeutic agent are administered substantially simultaneously, i.e. the compounds are administered at the same time or one after the other, so long as the compounds reach therapeutic levels in the blood at the same time. On another embodiment, the compound of the invention and at least one known cancer chemotherapeutic agent are administered according to their individual dose schedule, so long as the compounds reach therapeutic levels in the blood.

It has been reported that alpha-1-adrenoceptor antagonists, such as doxazosin, terazosin, and tamsulosin can inhibit the growth of prostate cancer cell via induction of apoptosis (Kyprianou, N., et al., Cancer Res 60:4550-4555, (2000)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known alpha-1-adrenoceptor antagonists, or a pharmaceutically acceptable salt of said agent. Examples of known alpha-1-adrenoceptor antagonists, which can be used for combination therapy include, but are not limited to, doxazosin, terazosin, and tamsulosin.

It has been reported that sigma-2 receptors are expressed in high densities in a variety of tumor cell types (Vilner, B. J., et al., Cancer Res. 55: 408-413 (1995)) and that sigma-2 receptor agonists, such as CB-64D, CB-184 and haloperidol activate a novel apoptotic pathway and potentiate antineoplastic drugs in breast tumor cell lines. (Kyprianou, N., et al., Cancer Res. 62:313-322 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known sigma-2 receptor agonist, or a pharmaceutically acceptable salt of said agonist. Examples of known sigma-2 receptor agonists which can be used for combination therapy include, but are not limited to, CB-64D, CB-184 and haloperidol.

It has been reported that combination therapy with lovastatin, a HMG-CoA reductase inhibitor, and butyrate, an inducer of apoptosis in the Lewis lung carcinoma model in mice, showed potentiating antitumor effects (Giermasz, A., et al., Int. J. Cancer 97:746-750 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known HMG-CoA reductase inhibitor, or a pharmaceutically acceptable salt of said agent. Examples of known HMG-CoA reductase inhibitors, which can be used for combination therapy include, but are not limited to, lovastatin, simvastatin, pravastatin, fluvastatin, atorvastatin and cerivastatin.

It has been reported that HIV protease inhibitors, such as indinavir or saquinavir, have potent anti-angiogenic activities and promote regression of Kaposi sarcoma (Sgadari, C., et al., Nat. Med. 8:225-232 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known HIV protease inhibitor, or a pharmaceutically acceptable salt of said agent. Examples of known HIV protease inhibitors, which can be used for combination therapy include, but are not limited to, amprenavir, abacavir, CGP-73547, CGP-61755, DMP-450, indinavir, nelfinavir, tipranavir, ritonavir, saquinavir, ABT-378, AG 1776, and BMS-232,632.

It has been reported that synthetic retinoids, such as fenretinide (N-(4-hydroxyphenyl)retinamide, 4HPR), have good activity in combination with other chemotherapeutic agents, such as cisplatin, etoposide or paclitaxel in small-cell lung cancer cell lines (Kalemkerian, G. P., et al., Cancer Chemother. Pharmacol. 43:145-150 (1999)). 4HPR also was reported to have good activity in combination with gamma-radiation on bladder cancer cell lines (Zou, C., et al., Int. J. Oncol. 13:1037-1041 (1998)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known retinoid and synthetic retinoid, or a pharmaceutically acceptable salt of said agent. Examples of known retinoids and synthetic retinoids, which can be used for combination therapy include, but are not limited to, bexarotene, tretinoin, 13-cis-retinoic acid, 9-cis-retinoic acid, α-difluoromethylomithine, ILX23-7553, fenretinide, and N-4-carboxyphenyl retinamide.

It has been reported that proteasome inhibitors, such as lactacystin, exert anti-tumor activity in vivo and in tumor cells in vitro, including those resistant to conventional chemotherapeutic agents. By inhibiting NF-kappaB transcriptional activity, proteasome inhibitors may also prevent angiogenesis and metastasis in vivo and further increase the sensitivity of cancer cells to apoptosis (Almond, J. B., et al., Leukemia 16:433-443 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known proteasome inhibitor, or a pharmaceutically acceptable salt of said agent. Examples of known proteasome inhibitors, which can be used for combination therapy include, but are not limited to, lactacystin, MG-132, and PS-341.

It has been reported that tyrosine kinase inhibitors, such as STI571 (Imatinib mesilate, Gleevec®), have potent synergetic effect in combination with other anti-leukemic agents, such as etoposide (Liu, W. M., et al. Br. J. Cancer 86:1472-1478 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known tyrosine kinase inhibitor, or a pharmaceutically acceptable salt of said agent. Examples of known tyrosine kinase inhibitors, which can be used for combination therapy include, but are not limited to, Gleevec®, ZD1839 (Iressa), SH268, genistein, CEP2563, SU6668, SU11248, and EMD121974.

It has been reported that prenyl-protein transferase inhibitors, such as farnesyl protein transferase inhibitor R115777, possess preclinical antitumor activity against human breast cancer (Kelland, L. R., et. al., Clin. Cancer Res. 7:3544-3550 (2001)). Synergy of the protein farnesyltransferase inhibitor SCH66336 and cisplatin in human cancer cell lines also has been reported (Adjei, A. A., et al., Clin. Cancer. Res. 7:1438-1445 (2001)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known prenyl-protein transferase inhibitor, including farnesyl protein transferase inhibitor, inhibitors of geranylgeranyl-protein transferase type I (GGPTase-I) and geranylgeranyl-protein transferase type-II, or a pharmaceutically acceptable salt of said agent. Examples of known prenyl-protein transferase inhibitors, which can be used for combination therapy include, but are not limited to, R115777, SCH66336, L-778,123, BAL9611 and TAN-1813.

It has been reported that cyclin-dependent kinase (CDK) inhibitors, such as flavopiridol, have potent synergetic effect in combination with other anticancer agents, such as CPT-11, a DNA topoisomerase I inhibitor in human colon cancer cells (Motwani, M., et al., Clin. Cancer Res. 7:4209-4219, (2001)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known cyclin-dependent kinase inhibitor, or a pharmaceutically acceptable salt of said agent. Examples of known cyclin-dependent kinase inhibitor, which can be used for combination therapy include, but are not limited to, flavopiridol, UCN-01, roscovitine and olomoucine.

It has been reported that in preclinical studies COX-2 inhibitors were found to block angiogenesis, suppress solid tumor metastases, and slow the growth of implanted gastrointestinal cancer cells (Blanke, C. D., Oncology (Huntingt) 16 (No. 4 Suppl. 3):17-21 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known COX-2 inhibitor, or a pharmaceutically acceptable salt of said inhibitor. Examples of known COX-2 inhibitors which can be used for combination therapy include, but are not limited to, celecoxib, valecoxib, and rofecoxib.

Another embodiment of the present invention is directed to a composition effective to inhibit neoplasia comprising a bioconjugate of a compound described herein, which functions as a caspase cascade activator and inducer of apoptosis, in bioconjugation with at least one known therapeutically useful antibody, such as Herceptin® or Rituxan®, growth factors, such as DGF, NGF; cytokines, such as IL-2, IL-4, or any molecule that binds to the cell surface. The antibodies and other molecules will deliver a compound described herein to its targets and make it an effective anticancer agent. The bioconjugates could also enhance the anticancer effect of therapeutically useful antibodies, such as Herceptin® or Rituxan®.

Similarly, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with radiation therapy. In this embodiment, the compound of the invention may be administered at the same time as the radiation therapy is administered or at a different time.

Yet another embodiment of the present invention is directed to a composition effective for post-surgical treatment of cancer, comprising a compound, or a pharmaceutically acceptable salt or prodrug of a compound described herein, which functions as a caspase cascade activator and inducer of apoptosis. The invention also relates to a method of treating cancer by surgically removing the cancer and then treating the animal with one of the pharmaceutical compositions described herein.

A wide range of immune mechanisms operates rapidly following exposure to an infectious agent. Depending on the type of infection, rapid clonal expansion of the T and B lymphocytes occurs to combat the infection. The elimination of the effector cells following an infection is one of the major mechanisms for maintaining immune homeostasis. The elimination of the effector cells has been shown to be regulated by apoptosis. Autoimmune diseases have lately been determined to occur as a consequence of deregulated cell death. In certain autoimmune diseases, the immune system directs its powerful cytotoxic effector mechanisms against specialized cells, such as oligodendrocytes in multiple sclerosis, the beta cells of the pancreas in diabetes mellitus, and thyrocytes in Hashimoto's thyroiditis (Ohsako, S. & Elkon, K. B., Cell Death Differ. 6:13-21 (1999)). Mutations of the gene encoding the lymphocyte apoptosis receptor Fas/APO-1/CD95 are reported to be associated with defective lymphocyte apoptosis and autoimmune lymphoproliferative syndrome (ALPS), which is characterized by chronic, histologically benign splenomegaly, generalized lymphadenopathy, hypergammaglobulinemia, and autoantibody formation. (Infante, A. J., et al., J. Pediatr. 133:629-633 (1998) and Vaishnaw, A. K., et al., J. Clin. Invest. 103:355-363 (1999)). It was reported that overexpression of Bcl-2, which is a member of the bcl-2 gene family of programmed cell death regulators with anti-apoptotic activity, in developing B cells of transgenic mice, in the presence of T cell dependent costimulatory signals, results in the generation of a modified B cell repertoire and in the production of pathogenic autoantibodies (Lopez-Hoyos, M., et al., Int. J. Mol. Med. 1:475-483 (1998)). It is therefore evident that many types of autoimmune disease are caused by defects of the apoptotic process. One treatment strategy for such diseases is to turn on apoptosis in the lymphocytes that are causing the autoimmune disease (O'Reilly, L. A. & Strasser, A., Inflamm. Res. 48:5-21 (1999)).

Fas-Fas ligand (FasL) interaction is known to be required for the maintenance of immune homeostasis. Experimental autoimmune thyroiditis (EAT), characterized by autoreactive T and B cell responses and a marked lymphocytic infiltration of the thyroid, is a good model to study the therapeutic effects of FasL. Batteux, F., et al., (J. Immunol. 162:603-608 (1999)) reported that by direct injection of DNA expression vectors encoding FasL into the inflamed thyroid, the development of lymphocytic infiltration of the thyroid was inhibited and induction of infiltrating T cells death was observed. These results show that FasL expression on thyrocytes may have a curative effect on ongoing EAT by inducing death of pathogenic autoreactive infiltrating T lymphocytes.

Bisindolylmaleimide VIII is known to potentiate Fas-mediated apoptosis in human astrocytoma 1321N1 cells and in Molt-4T cells; both of which were resistant to apoptosis induced by anti-Fas antibody in the absence of bisindolylmaleimide VIII. Potentiation of Fas-mediated apoptosis by bisindolylmaleimide VIII was reported to be selective for activated, rather than non-activated, T cells, and was Fas-dependent. Zhou T., et al., (Nat. Med. 5:42-48 (1999)) reported that administration of bisindolylmaleimide VIII to rats during autoantigen stimulation prevented the development of symptoms of T cell-mediated autoimmune diseases in two models, the Lewis rat model of experimental allergic encephalitis and the Lewis adjuvant arthritis model. Therefore, the application of a Fas-dependent apoptosis enhancer, such as bisindolylmaleimide VIII, may be therapeutically useful for the more effective elimination of detrimental cells and inhibition of T cell-mediated autoimmune diseases. Therefore, an effective amount of a compound, or a pharmaceutically acceptable salt or prodrug of the compound of Formulae I-V, which functions as a caspase cascade activator and inducer of apoptosis, is an effective treatment for autoimmune diseases.

Psoriasis is a chronic skin disease that is characterized by scaly red patches. Psoralen plus ultraviolet A (PUVA) is a widely used and effective treatment for psoriasis vulgaris. Coven, et al., Photodermatol. Photoimmunol. Photomed. 15:22-27 (1999), reported that lymphocytes treated with psoralen 8-MOP or TMP and UVA, displayed DNA degradation patterns typical of apoptotic cell death. Ozawa, et al., J. Exp. Med. 189:711-718 (1999) reported that induction of T cell apoptosis could be the main mechanism by which 312-nm UVB resolves psoriasis skin lesions. Low doses of methotrexate may be used to treat psoriasis to restore a clinically normal skin. Heenen, et al., Arch. Dermatol. Res. 290:240-245 (1998), reported that low doses of methotrexate may induce apoptosis and that this mode of action could explain the reduction in epidermal hyperplasia during treatment of psoriasis with methotrexate. Therefore, an effective amount of a compound, or a pharmaceutically acceptable salt or prodrug of the compound of Formulae I-V, which functions as a caspase cascade activator and inducer of apoptosis, is an effective treatment for hyperproliferative skin diseases, such as psoriasis.

Synovial cell hyperplasia is a characteristic of patients with rheumatoid arthritis (RA). It is believed that excessive proliferation of RA synovial cells, as well as defects in synovial cell death, may be responsible for synovial cell hyperplasia. Wakisaka, et al., Clin. Exp. Immunol. 114:119-128 (1998), found that although RA synovial cells could die via apoptosis through a Fas/FasL pathway, apoptosis of synovial cells was inhibited by proinflammatory cytokines present within the synovium. Wakisaka, et al. also suggested that inhibition of apoptosis by the proinflammatory cytokines may contribute to the outgrowth of synovial cells, and lead to pannus formation and the destruction of joints in patients with RA. Therefore, an effective amount of a compound, or a pharmaceutically acceptable salt or prodrug of the compound of Formulae I-V, which functions as a caspase cascade activator and inducer of apoptosis, is an effective treatment for rheumatoid arthritis.

There has been an accumulation of convincing evidence that apoptosis plays a major role in promoting resolution of the acute inflammatory response. Neutrophils are constitutively programmed to undergo apoptosis, thus limiting their pro-inflammatory potential and leading to rapid, specific, and non-phlogistic recognition by macrophages and semi-professional phagocytes (Savill, J., J. Leukoc. Biol. 61:375-380 (1997)). Boirivant, et al., Gastroenterology 116:557-565 (1999), reported that lamina propria T cells, isolated from areas of inflammation in Crohn's disease, ulcerative colitis, and other inflammatory states, manifest decreased CD2 pathway-induced apoptosis. In addition, studies of cells from inflamed Crohn's disease tissue indicate that this defect is accompanied by elevated Bcl-2 levels. Therefore, an effective amount of a compound, or a pharmaceutically acceptable salt or prodrug of the compound of Formulae I-V, which functions as a caspase cascade activator and inducer of apoptosis, is an effective treatment for inflammation.

Caspase cascade activators and inducers of apoptosis may also be a desirable therapy in the elimination of pathogens, such as HIV, Hepatitis C and other viral pathogens. The long lasting quiescence, followed by disease progression, may be explained by an anti-apoptotic mechanism of these pathogens leading to persistent cellular reservoirs of the virions. It has been reported that HIV-1 infected T leukemia cells or peripheral blood mononuclear cells (PBMCs) underwent enhanced viral replication in the presence of the caspase inhibitor Z-VAD-fmk. Furthermore, Z-VAD-fmk also stimulated endogenous virus production in activated PBMCs derived from HIV-1-infected asymptomatic individuals (Chinnaiyan, A., et al., Nat. Med. 3:333 (1997)). Therefore, apoptosis serves as a beneficial host mechanism to limit the spread of HIV and new therapeutics using caspase/apoptosis activators are useful to clear viral reservoirs from the infected individuals. Similarly, HCV infection also triggers anti-apoptotic mechanisms to evade the host's immune surveillance leading to viral persistence and hepatocarcinogenesis (Tai, D. I., et al. Hepatology 3:656-64 (2000)). Therefore, apoptosis inducers are useful as therapeutics for HIV and other infectious disease.

Stent implantation has become the new standard angioplasty procedure. However, in-stent restenosis remains the major limitation of coronary stenting. New approaches have been developed to target pharmacological modulation of local vascular biology by local administration of drugs. This allows for drug applications at the precise site and time of vessel injury. Numerous pharmacological agents with antiproliferative properties are currently under clinical investigation, including actinomycin D, rapamycin or paclitaxel coated stents (Regar E., et al., Br. Med. Bull. 59:227-248 (2001)). Therefore, apoptosis inducers, which are antiproliferative, are useful as therapeutics for the prevention or reduction of in-stent restenosis.

Pharmaceutical compositions within the scope of this invention include all compositions wherein the compounds of the present invention are contained in an amount that is effective to achieve its intended purpose. While individual needs vary, determination of optimal ranges of effective amounts of each component is within the skill of the art. Typically, the compounds may be administered to animals, e.g., mammals, orally at a dose of 0.0025 to 50 mg/kg of body weight, per day, or an equivalent amount of the pharmaceutically acceptable salt thereof, to a mammal being treated. Preferably, approximately 0.01 to approximately 10 mg/kg of body weight is orally administered. For intramuscular injection, the dose is generally approximately one-half of the oral dose. For example, a suitable intramuscular dose would be approximately 0.0025 to approximately 25 mg/kg of body weight, and most preferably, from approximately 0.01 to approximately 5 mg/kg of body weight. If a known cancer chemotherapeutic agent is also administered, it is administered in an amount that is effective to achieve its intended purpose. The amounts of such known cancer chemotherapeutic agents effective for cancer are well known to those skilled in the art.

The unit oral dose may comprise from approximately 0.01 to approximately 50 mg, preferably approximately 0.1 to approximately 10 mg of the compound of the invention. The unit dose may be administered one or more times daily, as one or more tablets, each containing from approximately 0.1 to approximately 10 mg, conveniently approximately 0.25 to 50 mg of the compound or its solvates.

In a topical formulation, the compound may be present at a concentration of approximately 0.01 to 100 mg per gram of carrier.

In addition to administering the compound as a raw chemical, the compounds of the invention may be administered as part of a pharmaceutical preparation containing suitable pharmaceutically acceptable carriers comprising excipients and auxiliaries, which facilitate processing of the compounds into preparations that may be used pharmaceutically. Preferably, the preparations, particularly those preparations which may be administered orally and that may be used for the preferred type of administration, such as tablets, dragees, and capsules, and also preparations that may be administered rectally, such as suppositories, as well as suitable solutions for administration by injection or orally, contain from approximately 0.01 to 99 percent, preferably from approximately 0.25 to 75 percent of active compound(s), together with the excipient.

Also included within the scope of the present invention are the non-toxic pharmaceutically acceptable salts of the compounds of the present invention. Acid addition salts are formed by mixing a solution of the compounds of the present invention with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Basic salts are formed by mixing a solution of the compounds of the present invention with a solution of a pharmaceutically acceptable non-toxic base, such as sodium hydroxide, potassium hydroxide, choline hydroxide, sodium carbonate, Tris, N-methyl-glucamine and the like.

The pharmaceutical compositions of the invention may be administered to any animal, which may experience the beneficial effects of the compounds of the invention. Foremost among such animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner, which is itself known, e.g., by means of conventional mixing, granulating, dragee-making, dissolving, or lyophilizing processes. Thus, pharmaceutical preparations for oral use may be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular: fillers, such as saccharides, e.g. lactose or sucrose, mannitol or sorbitol; cellulose preparations and/or calcium phosphates, e.g. tricalcium phosphate or calcium hydrogen phosphate; as well as binders, such as starch paste, using, e.g., maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, e.g., silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropylmethyl-cellulose phthalate, are used. Dye stuffs or pigments may be added to the tablets or dragee coatings, e.g., for identification or in order to characterize combinations of active compound doses.

Other pharmaceutical preparations, which may be used orally include push-fit capsules made of gelatin, as well as soft, sealed capsules made of gelatin and a plasticizer, such as glycerol or sorbitol. The push-fit capsules may contain the active compounds in the form of: granules, which may be mixed with fillers, such as lactose; binders, such as starches; and/or lubricants, such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations, which may be used rectally include, e.g., suppositories, which consist of a combination of one or more of the active compounds with a suppository base. Suitable suppository bases are, e.g., natural or synthetic triglycerides, or paraffin hydrocarbons. In addition, it is also possible to use gelatin rectal capsules, which consist of a combination of the active compounds with a base. Possible base materials include, e.g., liquid triglycerides, polyethylene glycols, or paraffin hydrocarbons.

Suitable formulations for parenteral administration include aqueous solutions of the active compounds in water-soluble form, e.g., water-soluble salts and alkaline solutions. In addition, suspensions of the active compounds as appropriate oily injection suspensions may be administered. Suitable lipophilic solvents or vehicles include fatty oils, e.g., sesame oil, or synthetic fatty acid esters, e.g., ethyl oleate or triglycerides or polyethylene glycol-400 (the compounds are soluble in PEG-400), or cremophor, or cyclodextrins. Aqueous injection suspensions may contain substances which increase the viscosity of the suspension include, e.g., sodium carboxymethyl cellulose, sorbitol, and/or dextran. Optionally, the suspension may also contain stabilizers.

In accordance with one aspect of the present invention, compounds of the invention are employed in topical and parenteral formulations and are used for the treatment of skin cancer.

The topical compositions of this invention are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included, as well as agents imparting color or fragrance, if desired. Additionally, transdermal penetration enhancers may be employed in these topical formulations. Examples of such enhancers are found in U.S. Pat. Nos. 3,989,816 and 4,444,762.

Creams are preferably formulated from a mixture of mineral oil, self-emulsifying beeswax and water in which mixture of the active ingredient, dissolved in a small amount of an oil, such as almond oil, is admixed. A typical example of such a cream is one which includes approximately 40 parts water, approximately 20 parts beeswax, approximately 40 parts mineral oil and approximately 1 part almond oil.

Ointments may be formulated by mixing a solution of the active ingredient in a vegetable oil, such as almond oil, with warm soft paraffin and allowing the mixture to cool. A typical example of such an ointment is one which includes approximately 30% almond oil and approximately 70% white soft paraffin by weight.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### EXAMPLE 1

### 4-Amino-5-(3,5-dimethoxyphenyl)-3-mercapto-(4H)-1,2,4-triazole

A mixture of 3,5-dimethoxybenzoic acid (1.82 g, 10 mmol) and thiocarbohydrazide (1.06 g, 10 mmol) was heated under argon at 180-185° C. until melting occurred, and it was stirred at 185° C. for 15 min. The reaction mixture was cooled, mixed with water (50 mL) and acidified with concentrated hydrochloric acid. The precipitate was filtered, washed with water, dried to give 2.4 g of solid product, which was used for next step without further purification.

### EXAMPLE 2

### 3-(3,5-Dimethoxyphenyl)-6-(3,4-methylenedioxyphenyl)-7H-[1,2,4]triazolo[3,4-b][1,3,4]thiadiazine

A mixture of 2-bromo-1-(3,4-methylenedioxyphenyl)ethanone (365 mg, 1.5 mmol) and 4-amino-5-(3,5-dimethoxyphenyl)-3-mercapto-(4H)-1,2,4-triazole HCl salt in ethanol (10 mL) was refluxed for 4 h. It was cooled and neutralized with aqueous sodium carbonate. The precipitate was collected and purified by flash chromatography (EtOAc/Hexane 7:1) to give 52 mg (10%) of the title compound. ¹H NMR (CDCl₃): 7.50 (d, J=1.5 Hz, 1H), 7.40-7.37 (dd, J=8.1, 1.5 Hz, 1H), 7.33 (d, J=2.4 Hz, 2H), 6.92 (d, J=8.1 Hz, 1H), 6.59 (t, J=2.4 Hz, 1H), 6.08 (s, 2H), 3.95 (s, 2H), 3.85 (s, 6H).

### EXAMPLE 3

### 4-Amino-5-(3-chloro-4,5-dimethoxyphenyl)-3-mercapto-(4H)-1,2,4-triazole

The title compound was prepared in a manner similar to example 1. From 3-chloro-4,5-dimethoxybenzoic acid (2.16 g, 10 mmol) and thiocarbohydrazide (1.06 g, 10 mmol) was obtained 2.75 g of solid without further purification.

### EXAMPLE 4

### 3-(3-Chloro-4,5-dimethoxyphenyl)-6-(3,4-methylenedioxyphenyl)-7H-[1,2,4]triazolo[3,4-b][1,3,4]thiadiazine

The title compound was prepared in a manner similar to example 2. From 2-bromo-1-(3,4-methylenedioxyphenyl)ethanone (486 mg, 2 mmol) and 4-amino-5-(3-chloro-4,5-dimethoxyphenyl)-3-mercapto-(4H)-1,2,4-triazole HCl salt (572 mg, 1.77 mmol) was obtained 70 mg (9.2%) of the title compound. ¹H NMR (CDCl₃): 7.80 (bs, 1H), 7.73 (d, J=1.8 Hz, 1H), 7.49 (d, J=2.1 Hz, 1H), 7.41-7.38 (dd, J=8.1, 1.8, 1H), 6.94 (d, J=8.4 Hz, 1H), 6.10 (s, 2H), 3.96 (s, 2H), 3.95 (s, 6H).

### EXAMPLE 5

### 3-(3-Chloro-4,5-dimethoxyphenyl)-6-(4-methoxyphenyl)-7H-[1,2,4]triazolo[3,4-b][1,3,4]thiadiazine

The title compound was prepared in a manner similar to example 2. From 2-bromo-1-(4-methoxyphenyl)ethanone (170 mg, 0.81 mmol) and 4-amino-5-(3-chloro-4,5-dimethoxyphenyl)-3-mercapto-(4H)-1,2,4-triazole HCl salt (263 mg, 0.81 mmol) was obtained 98 mg (29%) of the title compound. ¹H NMR (CDCl₃): 7.91 (d, J=9.0 Hz, 2H), 7.85 (bs, 1H), 7.74 (bs, 1H), 7.04 (d, J=9.0 Hz, 1H), 3.98 (s, 2H), 3.95 (s, 6H), 3.91 (s, 3H).

### EXAMPLE 6

### 3-(4-Chlorophenyl)-6-(4-hydroxyphenyl)-7H-[1,2,4]triazolo[3,4-b][1,3,4]thiadiazine hydrobromide

A mixture of 2-bromo-1-(4-hydroxyphenyl)ethanone (645 mg, 3.0 mmol) and 4-amino-5-(4-chlorophenyl)-3-mercapto-(4H)-1,2,4-triazole (681 mg, 3.0 mmol) in isopropanol was refluxed for 4 h and was then cooled to room temperature (rt). The precipitated solid was collected through filtration and dried to give 940 mg (74%) of the title compound. ¹H NMR (CD₃OD): 8.08 (d, J=8.7 Hz, 2H), 7.96 (d, J=9.0 Hz, 2H), 7.69 (d, J=8.7 Hz, 2H), 6.96 (d, J=9.0 Hz, 2H), 4.44 (s, 2H).

### EXAMPLE 7

### 6-(4-Hydroxyphenyl)-3-(2-methoxyphenyl)-7H-[1,2,4]triazolo[3,4-b][1,3,4]thiadiazine hydrobromide

The title compound was prepared in a manner similar to example 6. From 2-bromo-1-(4-hydroxyphenyl)ethanone (323 mg, 1.5 mmol) and 4-amino-5-(2-methoxyphenyl)-3-mercapto-(4H)-1,2,4-triazole (333 mg, 1.5 mmol) in isopropanol was obtained 510 mg (81%) of the title compound. ¹H NMR (DMSO-d₆): 7.74 (d, J=8.7 Hz, 2H), 7.65 (t, J=7.5 Hz, 1H), 7.55 (d, J=7.5 Hz, 1H), 7.23 (d, J=7.5 Hz, 1H), 7.13 (t, J=7.5 Hz, 1H), 6.87 (d, J=8.7 Hz, 2H), 4.36 (s, 2H), 3.75 (s, 3H).

### EXAMPLE 8

### 3-(2-Methoxyphenyl)-6-(4-methylphenyl)-7H-[1,2,4]triazolo[3,4-b][1,3,4]thiadiazine hydrochloride

A mixture of 2-bromo-1-(4-methylphenyl)ethanone (1.12 g, 5.25 mmol) and 4-amino-5-(2-methoxyphenyl)-3-mercapto-(4H)-1,2,4-triazole (1.17 g, 5.25 mmol) in isopropanol was refluxed for 4 h. It was neutralized with saturated aqueous sodium carbonate (5 mL), and the precipitate was collected through filtration. The solids were dissolved in methanol (30 mL) and acidified with HCl in ether (2 M, 4 mL). The solution was evaporated and the residue was mixed with ether (20 mL). The precipitates were collected by filtration to give 1.7 g (83%) of the title compound. ¹H NMR (CD₃OD): 7.91 (d, J=7.8 Hz, 2H), 7.89 (d, J=7.8 Hz, 1H), 7.76 (t, J=7.8 Hz, 1H), 7.38 (d, J=7.8 Hz, 2H), 7.36 (d, J=7.8 Hz, 1H), 7.25 (t, J=7.8 Hz, 1H), 4.48 (s, 2H), 3.98 (s, 3H), 2.44 (s, 3H).

### EXAMPLE 9

### 3-(2-Methoxyphenyl)-6-(4-(2-dimethylaminoethoxy)phenyl)-7H-[1,2,4]triazolo[3,4-b][1,3,4]thiadiazine

A mixture of 3-(2-methoxyphenyl)-6-(4-hydroxyphenyl)-7H-[1,2,4]triazolo[3,4-b][1,3,4]thiadiazine hydrobromide (120 mg, 0.29 mmol), 2-chloro-N,N-dimethylethanamine hydrochloride (102 mg, 0.71 mmol), potassium carbonate (118 mg, 0.71 mmol) in acetone (20 mL) was refluxed for 8 h. It was evaporated, and the residue was diluted with water (30 mL) and extracted with ethyl acetate (3×20 mL). The organic extracts were dried, concentrated to give 54 mg (46%) of the title compound. ¹H NMR (CDCl₃): 7.74 (d, J=9.0 Hz, 2H), 7.652 (dd, J1=7.5 Hz, J2=1.5 Hz, 1H), 7.52 (m, 1H), 7.10 (dt, J1=7.5 Hz, J2=0.9 Hz, 1H), 7.02 (d, J=8.4 Hz, 1H), 6.96 (d, J=8.7 Hz, 2H), 4.11 (t, J=6.0 Hz, 2H), 3.95 (s, 2H), 3.75 (s, 3H), 2.75 (t, J=6.0 Hz, 2H), 2.34 (s, 6H).

### EXAMPLE 10

### 6-(4-(2-Hydroxyethoxy)phenyl)-3-(2-methoxyphenyl)-7H-[1,2,4]triazolo[3,4-b][1,3,4]thiadiazine

A mixture of 3-(2-methoxyphenyl)-6-(4-hydroxyphenyl)-7H-[1,2,4]triazolo[3,4-b][1,3,4]thiadiazine hydrobromide (120 mg, 0.29 mmol), 2-iodoethanol (197 mg, 1.14 mmol), potassium carbonate (188 mg, 1.14 mmol) in acetone (20 mL) was refluxed for 8 h. It was evaporated, and the residue was diluted with water (30 mL) and extracted with ethyl acetate (3×20 mL). The organic extracts were dried, concentrated and applied to column chromatography to give 36 mg (33%) of the title compound. ¹H NMR (CD₃OD): 7.87 (d, J=9.0 Hz, 2H), 7.58 (m, 1H), 7.10 (dd, J1=7.8 Hz, J2=2.1 Hz, 1H), 7.18 (d, J=8.1 Hz, 1H), 7.12 (dt, J1=7.2 Hz, J2=0.9 Hz, 1H), 7.04 (d, J=9.0 Hz, 2H), 4.28 (s, 2H), 4.11 (t, J=4.5 Hz, 2H), 3.88 (t, J=4.5 Hz, 2H), 3.80 (s, 3H).

### EXAMPLE 11

### 3-(2-Chlorophenyl)-6-(4-methylphenyl)-7H-[1,2,4]triazolo[3,4-b][1,3,4]thiadiazine hydrochloride

The title compound was prepared in a manner similar to example 8. From 2-bromo-1-(4-methylphenyl)ethanone (213 mg, 1.0 mmol) and 4-amino-5-(2-chlorophenyl)-3-mercapto-(4H)-1,2,4-triazole (227 mg, 1.0 mmol) in isopropanol was obtained 210 mg (53%) of the title compound. ¹H NMR (CDCl₃): 7.70 (d, J=8.1 Hz, 2H), 7.56-7.40 (m, 3H), 7.28-7.25 (m, 3H), 3.99 (s, 2H), 2.41 (s, 3H).

### EXAMPLE 12

### 3-(3,5-Dimethoxyphenyl)-6-(4-methylphenyl)-7H-[1,2,4]triazolo[3,4-b][1,3,4]thiadiazine hydrochloride

The title compound was prepared in a manner similar to example 8. From 2-bromo-1-(4-methylphenyl)ethanone (213 mg, 1.0 mmol) and 4-amino-5-(3,5-dimethoxyphenyl)-3-mercapto-(4H)-1,2,4-triazole (252 mg, 1.0 mmol) in isopropanol was obtained 221 mg (55%) of the title compound. ¹H NMR (CD₃OD): 8.09 (d, J=8.1 Hz, 2H), 7.54 (d, J=8.7 Hz, 2H), 7.41 (bs, 2H), 76.86 (bs, 1H), 4.55 (s, 2H), 4.00 (s, 3H), 2.59 (s, 3H).

### EXAMPLE 13

### 3-(3,5-Dimethoxy-4-hydroxyphenyl)-6-(4-methylphenyl)-7H-[1,2,4]triazolo[3,4-b][1,3,4]thiadiazine hydrobromide

The title compound was prepared in a manner similar to example 6. From 2-bromo-1-(4-methylphenyl)ethanone (213 mg, 1.0 mmol) and 4-amino-5-(3,5-dimethoxy-4-hydroxyphenyl)-3-mercapto-(4H)-1,2,4-triazole (282 mg, 1.0 mmol) in isopropanol was obtained 225 mg (49%) of the title compound. ¹H NMR (DMSO-d₆): 7.97 (d, J=8.1 Hz, 2H), 7.42 (d, J=8.1 Hz, 2H), 7.40 (s, 2H), 4.43 (s, 2H), 3.83 (s, 6H), 2.40 (s, 3H).

### EXAMPLE 14

### 3-(3,4-Dimethoxyphenyl)-6-(4-methylphenyl)-7H-[1,2,4]triazolo[3,4-b][1,3,4]thiadiazine hydrobromide

The title compound was prepared in a manner similar to example 6. From 2-bromo-1-(4-methylphenyl)ethanone (213 mg, 1.0 mmol) and 4-amino-5-(3,4-dimethoxyphenyl)-3-mercapto-(4H)-1,2,4-triazole (252 mg, 1.0 mmol) in isopropanol was obtained 201 mg (45%) of the title compound. ¹H NMR (DMSO-d₆): 7.93 (d, J=8.4 Hz, 2H), 7.64 (s, 1H), 7.65-7.61 (q, J1=8.1 Hz, J2=1.8 Hz, 1H), 7.40 (d, J=7.8 Hz, 2H), 7.16 (d, J=7.8 Hz, 1H), 4.42 (s, 2H), 3.84 (s, 3H), 3.83 (s, 3H), 2.40 (s, 3H).

### EXAMPLE 15

### 3-(2,4-Dimethoxyphenyl)-6-(4-methylphenyl)-7H-[1,2,4]triazolo[3,4-b][1,3,4]thiadiazine hydrobromide

The title compound was prepared in a manner similar to example 2. From 2-bromo-1-(4-methylphenyl)ethanone (213 mg, 1.0 mmol) and 4-amino-5-(2,4-dimethoxyphenyl)-3-mercapto-(4H)-1,2,4-triazole (252 mg, 1.0 mmol) in isopropanol was obtained 245 mg (67%) of the title compound. ¹H NMR (CDCl₃): 7.70 (d, J=8.4 Hz, 2H), 7.55 (d, J=8.4 Hz, 1H), 7.26 (d, J=8.4 Hz, 2H), 6.64-6.60 (q, J1=8.7 Hz, J2=2.1 Hz, 1H), 6.55 (d, J=2.1 Hz, 1H), 3.95 (s, 2H), 3.88 (s, 3H), 3.72 (s, 3H), 2.41 (s, 3H).

### EXAMPLE 16

### 3-(3,5-Dimethoxyphenyl-4-(2-dimethylaminoethoxy)phenyl)-6-(4-methylphenyl)-7H-[1,2,4]triazolo[3,4-b][1,3,4]thiadiazine

A mixture of 3-(3,5-dimethoxy-4-hydroxyphenyl)-6-(4-methylphenyl)-7H-[1,2,4]triazolo[3,4b][1,3,4]thiadiazine hydrobromide (80 mg, 0.21 mmol), 2-chloro-N,N-dimethylethanamine hydrochloride (60 mg, 0.42 mmol), potassium carbonate (116 mg, 0.84 mmol), and potassium iodide (70 mg, 0.42 mmol) in acetone (20 mL) was refluxed for 8 h. It was evaporated, and the residue was diluted with water (30 mL) and extracted with ethyl acetate (3×20 mL). The organic extracts were dried and concentrated, and the residue was purified by column chromatography (EtOAc/MeOH 10:1) to give 13 mg (14%) of the title compound. ¹H NMR (CDCl₃): 7.83 (d, J=8.4 Hz, 2H), 7.50 (s, 2H), 7.32 (d, J=8.4 Hz, 2H), 4.17 (t, J=6.3 Hz, 2H), 3.99 (s, 2H), 3.89 (s, 6H), 2.80 (t, J=6.3 Hz, 2H), 2.45 (s, 3H), 2.42 (s, 6H).

### EXAMPLE 17

### 3-(3,4,5-Trimethoxyphenyl)-6-(4-methylphenyl)-7H-[1,2,4]triazolo[3,4-b][1,3,4]thiadiazine

The title compound was prepared in a manner similar to example 16. From 3-(3,5-dimethoxy-4-hydroxyphenyl)-6-(4-methylphenyl)-7H-[1,2,4]triazolo[3,4-b][1,3,4]thiadiazine hydrobromide (80 mg, 0.21 mmol) and methyl iodide (59 mg, 2.0 mmol) was obtained 14 mg (17%) of the title compound. ¹H NMR (CDCl₃): 7.80 (d, J=8.1 Hz, 2H), 7.51 (s, 2H), 7.32 (d, J=8.1 Hz, 2H), 4.00 (s, 2H), 3.93 (s, 3H), 3.92 (s, 6H), 2.45 (s, 3H).

### EXAMPLE 18

### 3-(2,4-Dimethoxyphenyl)-6-(4-methoxyphenyl)-7H-[1,2,4]triazolo[3,4-b][1,3,4]thiadiazine hydrochloride

The title compound was prepared in a manner similar to example 8. From 2-bromo-1-(4-methoxyphenyl)ethanone (688 mg, 3.0 mmol) and 4-amino-5-(2,4-dimethoxyphenyl)-3-mercapto-(4H)-1,2,4-triazole (756 mg, 3.0 mmol) in isopropanol was obtained 1.14 g (91%) of the title compound. ¹H NMR (CD₃OD): 8.05 (d, J=9.3 Hz, 2H), 8.02 (d, J=9.0 Hz, 1H), 7.12 (d, J=9.0 Hz, 2H), 6.87 (s, 1H), 6.88-6.83 (m, 1H), 4.46 (s, 2H), 4.03 (s, 3H), 3.96 (s, 3H), 3.91 (s, 3H).

### EXAMPLE 19

### 3-(2-Ethoxyphenyl)-6-(4-methylphenyl)-7H-[1,2,4]triazolo[3,4-b][1,3,4]thiadiazine hydrobromide

The title compound was prepared in a manner similar to example 6. From 2-bromo-1-(4-methylphenyl)ethanone (213 mg, 1.0 mmol) and 4-amino-5-(2-ethoxyphenyl)-3-mercapto-(4H)-1,2,4-triazole (236 mg, 1.0 mmol) in isopropanol was obtained 1.06 g (100%) of the title compound. ¹H NMR (CD₃OD): 7.91 (d, J=8.1 Hz, 2H), 7.82 (dd, J=7.8, 1.8 Hz, 1H), 7.77-7.71 (m, 1H), 7.38 (d, J=8.1 Hz, 2H), 7.33 (d, J=8.1 Hz, 1H), 7.26-7.21 (m, 1H), 4.50 (s, 2H), 4.25 (q, J=7.2 Hz, 2H), 2.44 (s, 3H), 1.33 (t, J=7.2 Hz, 3H).

### EXAMPLE 20

### 3-(3,5-Dimethoxyphenyl)-6-(4-methoxyphenyl)-7H-[1,2,4]triazolo[3,4-b][1,3,4]thiadiazine hydrobromide

The title compound was prepared in a manner similar to example 6. From 2-bromo-1-(4-methoxyphenyl)ethanone (229 mg, 1.0 mmol) and 4-amino-5-(3,5-dimethoxyphenyl)-3-mercapto-(4H)-1,2,4-triazole (252 mg, 1.0 mmol) in isopropanol was obtained 0.32 g (69%) of the title compound. ¹H NMR (CD₃OD): 8.07 (d, J=9.0 Hz, 2H), 7.13 (d, J=9.0 Hz, 2H), 6.83 (t, J=2.4 Hz, 1H), 4.46 (s, 2H), 3.91 (s, 3H), 3.87 (s, 6H).

### EXAMPLE 21

### 6-(4-Diethylaminophenyl)-3-(3,4,5-trimethoxyphenyl)-7H-[1,2,4]triazolo[3,4-b][1,3,4]thiadiazine hydrochloride

The title compound was prepared in a manner similar to example 8. From 2-bromo-1-(4-diethylaminophenyl)ethanone (189 mg, 0.70 mmol) and 4-amino-5-(3,4,5-trimethoxyphenyl)-3-mercapto-(4H)-1,2,4-triazole (223 mg, 0.70 mmol) in isopropanol was obtained 0.26 g (77%) of the title compound. ¹H NMR (CD₃OD): 8.20 (d, J=8.7 Hz, 2H), 7.46 (s, 2H), 7.44 (d, J=8.7 Hz, 2H), 4.48 (s, 2H), 3.92 (s, 6H), 3.89 (s, 3H), 3.66 (q, J=6.9 Hz, 4H), 1.20 (t, J=6.9 Hz, 6H).

### EXAMPLE 22

### 6-(4-Diethylaminophenyl)-3-(3,4-dimethoxyphenyl)-7H-[1,2,4]triazolo[3,4-b][1,3,4]thiadiazine

A mixture of 2-bromo-1-(4-diethylaminophenyl)ethanone (470 mg, 1.74 mmol) and 4-amino-5-(3,4-dimethoxyphenyl)-3-mercapto-(4H)-1,2,4-triazole (402 mg, 1.59 mmol) in 15 mL of THF was heated in a sealed tube at 100° C. for 4 h. The mixture was cooled to rt, diluted with of ethyl acetate (150 mL), and washed with saturated NaHCO₃ (150 mL). The organic layer was dried over anhydrous Na₂SO₄, filtered and concentrated. The residue was purified by chromatography (90% ethyl acetate/hexane, 0.5 mL methanol/100 mL) to give the title compound (495 mg, 1.17 mmol, 73%). ¹H NMR (CDCl₃): 7.78-7.83 (m, 4H), 6.97 (d, J=9.0 Hz, 1H), 6.71 (m, 2H), 3.96 (s, 3H), 3.95 (s, 3H), 3.92 (s, 2H), 3.44 (q, J=6.9 Hz, 4H), 1.22 (t, J=7.2 Hz, 6H).

### EXAMPLE 23

### 6-(4-Diethylaminophenyl)-3-(2-methoxyphenyl)-7H-[1,2,4]triazolo[3,4-b][1,3,4]thiadiazine

A mixture of 2-bromo-1-(4-diethylaminophenyl)ethanone (130 mg, 0.48 mmol) and 4-amino-5-(2-methoxyphenyl)-3-mercapto-(4H)-1,2,4-triazole (100 mg, 0.45 mmol) in 1.5 mL of THF was refluxed for 4 h. The mixture was cooled to room temperature, diluted with 100 mL of ethyl acetate, and washed with saturated NaHCO₃ (50 mL). The organic layer was dried over anhydrous Na₂SO₄, filtered and concentrated. The residue was purified by chromatography (90% ethyl acetate/hexane, 0.5 mL methanol/100 mL). The NMR indicated that the product was a 1:1 mixture of noncyclized 2-(5-(2-methoxyphenyl)-4-amino-4H-1,2,4-triazol-3-ylthio)-1-(4-(diethylamino)phenyl)ethanone and the title compound. The mixture was suspended in 20 mL of toluene and few crystals of p-toluene sulfonic acid (PTSA) in a 50 mL round bottomed flask equipped with a Dean-Stark apparatus and refluxed for 3 h. The mixture was cooled to room temperature, diluted with 100 mL of ethyl acetate, and washed with saturated NaHCO₃ (50 mL). The organic layer was dried over anhydrous Na₂SO₄, filtered and concentrated. The residue was purified by chromatography (90% ethyl acetate/hexane, 0.5 mL methanol/100 mL) to give the title product (78 mg, 0.20 mmol, 44%). ¹H NMR (CDCl₃): 7.67 (m, 2H), 7.62 (dd, J=2.1, 7.2 Hz, 1H), 7.48 (m, 1H), 7.08 (dt, J=0.6 Hz, 7.5, 1H), 7.01 (d, J=8.4 Hz, 1H), 6.63 (m, 2H), 3.91 (s, 2H), 3.76 (s, 3H), 3.40 (q, J=7.2 Hz, 4H), 1.19 (t, J=6.9 Hz, 6H).

### EXAMPLE 24

### 3-(2-Methoxyphenyl)-6-(4-(pyrrolidin-1-yl)phenyl)-7H-[1,2,4]triazolo[3,4-b][1,3,4]thiadiazine

The title compound was prepared in a manner similar to example 23. From 2-bromo-1-(4-(pyrrolidin-1-yl)phenyl)ethanone (130 mg, 0.48 mmol) and 4-amino-5-(2-methoxyphenyl)-3-mercapto-(4H)-1,2,4-triazole (100 mg, 0.45 mmol) was obtained the title compound as a yellow solid (94 mg, 0.24 mmol, 53%). ¹H NMR (CDCl₃): 7.69 (m, 2H), 7.62 (dd, J=2.1, 7.2 Hz, 1H), 7.48 (m, 1H), 7.08 (m, 1H), 7.01 (d, J=8.1 Hz, 1H), 6.53 (m, 2H), 3.91 (s, 2H), 3.78 (s, 3H), 3.34 (m, 2H), 2.02 (m, 2H).

### EXAMPLE 25

### 3-(2-Methoxyphenyl)-6-(4-morpholinophenyl)-7H-[1,2,4]triazolo[3,4-b][1,3,4]thiadiazine

The title compound was prepared in a manner similar to example 23. From 2-bromo-1-(4-morpholinophenyl)ethanone (135 mg, 0.48 mmol) and 4-amino-5-(2-methoxyphenyl)-3-mercapto-(4H)-1,2,4-triazole (100 mg, 0.45 mmol) was obtained the title compound as a yellow solid (71 mg, 0.17 mmol, 39%). ¹H NMR (CDCl₃): 8.87 (m, 2H), 7.76 (dd, J=1.5, 7.5 Hz, 1H), 7.63 (dt, J=1.8, 7.5 Hz, 1H), 7.23 (dt, J=0.9, 7.5 Hz, 1H), 7.16 (d, J=8.4 Hz, 1H), 7.02 (m, 2H), 4.08 (s, 2H), 4.00 (m, 2H), 3.89 (s, 3H), 3.41 (m, 2H).

### EXAMPLE 26

### 6-(4-Diethylaminophenyl)-3-(3,5-dimethoxyphenyl)-7H-[1,2,4]triazolo[3,4-b][1,3,4]thiadiazine

The title compound was prepared in a manner similar to example 22. From 2-bromo-1-(4-diethylaminophenyl)ethanone (175 mg, 0.65 mmol) and 4-amino-5-(3,5-dimethoxyphenyl)-3-mercapto-(4H)-1,2,4-triazole (150 mg, 0.59 mmol) was obtained the title compound as a yellow solid (101 mg, 0.24 mmol, 40%). ¹H NMR (CDCl₃): 8.81 (m, 2H), 7.41 (d, J=2.4 Hz, 2H), 6.70 (m, 2H), 6.58 (t, J=2.4 Hz, 1H), 3.91 (s, 2H), 3.85 (s, 3H), 3.43 (q, J=7.2 Hz, 4H), 1.22 (t, J=7.2 Hz, 6H).

### EXAMPLE 27

### 6-(4-Diethylaminophenyl)-3-(2,4-dimethoxyphenyl)-7H-[1,2,4]triazolo[3,4-b][1,3,4]thiadiazine

The title compound was prepared in a manner similar to example 22. From 2-bromo-1-(4-diethylaminophenyl)ethanone (175 mg, 0.65 mmol) and 4-amino-5-(2,4-dimethoxyphenyl)-3-mercapto-(4H)-1,2,4-triazole (150 mg, 0.59 mmol) was obtained the title compound as a yellow solid (198 mg, 0.47 mmol, 79%). ¹H NMR (CDCl₃): 7.68 (m, 2H), 7.54 (d, J=8.7 Hz, 1H), 6.55-6.66 (m, 4H), 3.89 (s, 3H), 3.88 (s, 3H), 3.38 (q, J=6.9 Hz, 4H), 1.19 (t, J=6.9 Hz, 6H).

### EXAMPLE 28

### 3-(3,4-Dimethoxyphenyl)-6-(4-nitrophenyl)-7H-[1,2,4]triazolo[3,4-b][1,3,4]thiadiazine

The title compound was prepared in a manner similar to example 22. From 2-bromo-1-(4-nitrophenyl)ethanone (320 mg, 1.3 mmol) and 4-amino-5-(3,4-dimethoxyphenyl)-3-mercapto-(4H)-1,2,4-triazole (300 mg, 1.2 mmol) was obtained the title compound as a yellow solid (258 mg, 0.65 mmol, 55%). ¹H NMR (CDCl₃): 8.38 (m, 2H), 8.11 (m, 2H), 7.72 (d, J=1.8 Hz, 1H), 7.63 (dd, J=1.8, 8.1 Hz, 1H), 6.98 (d, J=8.4 Hz, 1H), 4.07 (s, 2H), 3.96 (s, 3H), 3.95 (s, 3H).

### EXAMPLE 29

### 6-(4-Aminophenyl)-3-(3,4-dimethoxyphenyl)-7H-[1,2,4]triazolo[3,4-b][1,3,4]thiadiazine

A mixture of 3-(3,4-dimethoxyphenyl)-6-(4-nitrophenyl)-7H-[1,2,4]triazolo[3,4-b][1,3,4]thiadiazine (241 mg, 0.61 mmol) in THF (10 mL), methanol (20 mL) and HCl (1N, 1 mL) was hydrogenated at 60 psi for 6 h using Palladium on carbon (5% Pd content, 80 mg). The resulting mixture was filtered through a pad of Celite and the filtrate was concentrated under vacuum to obtain the title compound (238 mg, 0.59 mmol, 97%). ¹H NMR (DMSO-d₆): 7.84-7.92 (m, 2H), 7.68 (m, 2H), 7.19 (d, J=8.7 Hz, 1H), 6.85-6.94 (m, 2H), 4.57 (bs, 1H), 4.32 (s, 2H), 3.85 (s, 3H), 3.83 (s, 3H).

### EXAMPLE 30

### 6-(4-Dimethylminophenyl)-3-(3,4-dimethoxyphenyl)-7H-[1,2,4]triazolo[3,4-b][1,3,4]thiadiazine

To a solution of 6-(4-aminophenyl)-3-(3,4-dimethoxyphenyl)-7H-[1,2,4]triazolo[3,4-b][1,3,4]thiadiazine (50 mg, 0.12 mmol) in methanol (4 mL) was added formaldehyde (35% aqueous, 1 mL). The reaction mixture was cooled to 0° C., then was added sodium cyanoborohydride (25 mg×3). The reaction mixture was stirred at 0° C. for 1 h and warmed to room temperature. The mixture was diluted with 50 mL of ethyl acetate and washed with saturated NaHCO₃ (50 mL). The organic layer was dried over anhydrous Na₂SO₄, filtered and concentrated. The residue was purified by chromatography (4% isopropanol/dichloromethane) to give the title compound (24 mg, 0.061 mmol, 49%). ¹H NMR (CDCl₃): 8.81-7.85 (m, 4H), 6.97 (d, J=8.7 Hz, 1H), 6.73 (m, 2H), 3.96 (s, 3H), 3.95 (s, 3H), 3.93 (s, 2H), 3.08 (s, 6H).

### EXAMPLE 31

### 6-(4-Azidophenyl)-3-(3,4-dimethoxyphenyl)-7H-[1,2,4]triazolo[3,4-b][1,3,4]thiadiazine

An aqueous solution of NaNO₂ (30 mg, 0.43 mmol, in 250 μL water) was added dropwise at 0° C. to a solution of 6-(4-aminophenyl)-3-(3,4-dimethoxyphenyl)-7H-[1,2,4]triazolo[3,4-b][1,3,4]thiadiazine (50 mg, 0.12 mmol) in methanol (4 mL) and HCl (1N, 2 drops). The mixture was stirred at the same temperature for 15 min, then an aqueous solution of NaN₃ (30 mg, 0.46 mmol, in 250 μL water) was added and the solution was warmed to room temperature and stirred for 1 h. The mixture was diluted with 50 mL of ethyl acetate and washed with saturated NaHCO₃ (50 mL). The organic layer was dried over anhydrous Na₂SO₄, filtered and concentrated. The residue was purified by chromatography (90% ethyl acetate/hexane) to give the title compound (20 mg, 0.051 mmol, 41%). ¹H NMR (CDCl₃): 7.93 (m, 2H), 7.69-7.56 (m, 2H), 7.16 (m, 2H), 6.97 (d, J=8.7 Hz, 1H), 3.98 (s, 2H), 3.95 (s, 6H).

### EXAMPLE 32

### 6-(4-Acetamidophenyl)-3-(3,4-dimethoxyphenyl)-7H-[1,2,4]triazolo[3,4-b][1,3,4]thiadiazine

To a solution of 6-(4-aminophenyl)-3-(3,4-dimethoxyphenyl)-7H-[1,2,4]triazolo[3,4-b][1,3,4]thiadiazine (52 mg, 0.13 mmol) in THF (3 mL) at 0° C. was added acetic anhydride (200 μL, 2.1 mmol), triethylamine (500 μL, 3.6 mmol), and a few crystals of 4-(dimethylamino)pyridine (DMAP). The reaction mixture was stirred for 2 h and it was warmed to room temperature. The mixture was diluted with 50 mL of ethyl acetate and washed with saturated NaHCO₃ (50 mL). The organic layer was dried over anhydrous Na₂SO₄, filtered and concentrated. The residue was purified by chromatography (90% ethyl acetate/hexane) to obtain the title compound (19 mg, 0.046 mmol, 36%). ¹H NMR (CDCl₃): 7.90 (m, 2H), 7.69-7.76 (m, 4H), 7.52 (bs, 1H), 6.98 (d, J=8.4 Hz, 1H), 3.98 (s, 2H), 3.96 (s, 3H), 3.94 (s, 3H), 2.24 (s, 3H).

### EXAMPLE 33

### 6-(2,3-Dihydro-2-oxobenzo[d]oxazol-6-yl)-3-(2,4-dimethoxyphenyl)-7H-[1,2,4]triazolo[3,4-b][1,3,4]thiadiazine

(a) 6-(2-(5-(2,4-Dimethoxyphenyl)-4-amino-4H-1,2,4-triazol-3-ylthio)acetyl)benzo[d]oxazol-2(3H)-one. To an oven-dried round bottom reaction flask charged with a magnetic stir bar under argon at rt with 6-(2-chloroacetyl)benzo[d]oxazol-2(3H)-one (0.200 g, 0.945 mmol) and 4-amino-5-(2,4-dimethoxyphenyl)-3-mercapto-(4H)-1,2,4-triazole (0.238 g, 0.945 mmol) was added anhydrous isopropanol (4.7 mL). The resulting white suspension was heated to reflux for 2.5 h and then cooled to rt. The white suspension was diluted with isopropanol and then filtered through a Buchner funnel to give 0.356 g (92%) of the title compound as a white solid. ¹H NMR (DMSO-d₆): 12.19 (br s, 1H), 7.88-7.84 (m, 2H), 7.32 (d, J=8.4 Hz, 1H), 7.23 (d, J=8.1 Hz, 1H), 6.71-6.62 (m, 2H), 5.16 (s, 2H), 3.84 (s, 3H), 3.80 (s, 3H).

(b) 6-(2,3-Dihydro-2-oxobenzo[d]oxazol-6-yl)-3-(2,4-dimethoxyphenyl)-7H-[1,2,4]triazolo[3,4-b][1,3,4]thiadiazine. To an oven-dried one-neck reaction flask charged with a magnetic stir bar at rt under argon with a Dean-Stark apparatus was added 6-(2-(5-(2,4-dimethoxyphenyl)-4-amino-4H-1,2,4-triazol-3-ylthio)acetyl)benzo[d]oxazol-2(3H)-one (0.356 g, 0.833 mmol), p-toluenesulfonic acid (catalytic) and anhydrous toluene (8.5 mL). The white suspension was heated to 150° C. for 3 h and then cooled to rt. The resulting precipitate was filtered on a Buchner funnel to give 0.307 g of crude product as a white solid. It was purified by flash column chromatography (silica gel, elution with iPrOH:CH₂Cl₂, 5:95) to give 0.050 g (15%) of the title compound as a white solid. ¹H NMR (DMSO-d₆): 7.84-7.81 (m, 2H), 7.48 (d, J=8.4 Hz, 1H), 7.27 (d, J=8.1 Hz, 2H), 6.72-6.69 (m, 2H), 4.38 (s, 2H), 3.90 (s, 3H), 3.78 (s, 3H).

### EXAMPLE 34

### 6-(2,3-Dihydro-2-oxobenzo[d]oxazol-6-yl)-3-(3,5-dimethoxyphenyl)-7H-[1,2,4]triazolo[3,4-b][1,3,4]thiadiazine

(a) 6-(2-(5-(3,5-Dimethoxyphenyl)-4-amino-4H-1,2,4-triazol-3-ylthio)acetyl)benzo[d]oxazol-2(3H)-one. The title compound was prepared in a manner similar to example 33a. From 6-(2-chloroacetyl)benzo[d]oxazol-2(3H)-one (0.200 g, 0.945 mmol) and 4-amino-5-(3,5-dimethoxyphenyl)-3-mercapto-(4H)-1,2,4-triazole (0.238 g, 0.945 mmol) was obtained 0.302 g (78%) of the title compound as a white solid. ¹H NMR (DMSO-d₆): 12.16 (br s, 1H), 7.96-7.84 (m, 2H), 7.26-7.20 (m, 2H), 7.14-7.13 (m, 1H), 6.67-6.66 (m, 1H), 5.16 (s, 1H), 4.92 (s, 1H), 3.80 (s, 3H), 3.79 (s, 3H).

(b) 6-(2,3-Dihydro-2-oxobenzo[d]oxazol-6-yl)-3-(3,5-dimethoxyphenyl)-7H-[1,2,4]triazolo[3,4-b][1,3,4]thiadiazine. The title compound was prepared in a manner similar to example 33b. From 6-(2-(5-(3,5-dimethoxyphenyl)-4-amino-4H-1,2,4-triazol-3-ylthio)acetyl)benzo[d]oxazol-2(3H)-one (0.299 g, 0.699 mmol) was obtained 0.020 g (7%) of the title compound as a white solid. ¹H NMR (DMSO-d₆): 12.09 (br s, 1H), 7.93 (d, J=1.5 Hz, 1H), 7.87 (dd, J=8.4 Hz, 1H), 7.29 (d, J=8.4 Hz, 1H), 7.22 (d, J=2.6 Hz, 2H), 6.69 (t, J=2.6 Hz, 1H), 4.43 (s, 2H), 3.82 (s, 6H).

### EXAMPLE 35

### 6-(2,3-Dihydro-2-oxobenzo[d]oxazol-6-yl)-3-(3,4-dimethoxyphenyl)-7H-[1,2,4]triazolo[3,4-b][1,3,4]thiadiazine

(a) 6-(2-(5-(3,4-Dimethoxyphenyl)-4-amino-4H-1,2,4-triazol-3-ylthio)acetyl)-benzo[d]oxazol-2(3H)-one. The title compound was prepared in a manner similar to example 33a. From 6-(2-chloroacetyl)benzo[d]oxazol-2(3H)-one (0.200 g, 0.945 mmol) and 4-amino-5-(3,4-dimethoxyphenyl)-3-mercapto-(4H)-1,2,4-triazole (0.238 g, 0.945 mmol) was 0.050 g (13%) of the title compound as a white solid. ¹H NMR (DMSO-d₆): 13.82 (br s, 1H), 12.17 (br s, 1H), 7.87-7.83 (m, 2H), 7.66 (dd, J=8.4 and 1.8 Hz, 1H), 7.57 (d, J=1.8 Hz, 1H), 7.22 (d, J=8.1 Hz, 1H), 7.10 (d, J=8.8 Hz, 1H), 5.16 (s, 2H), 3.82 (s, 3H), 3.80 (s, 3H).

(b) 6-(2,3-Dihydro-2-oxobenzo[d]oxazol-6-yl)-3-(3,4-dimethoxyphenyl)-7H-[1,2,4]triazolo[3,4-b][1,3,4]thiadiazine. The title compound was prepared in a manner similar to example 33b. From 6-(2-(5-(3,4-dimethoxyphenyl)-4-amino-4H-1,2,4-triazol-3-ylthio)acetyl)-benzo[d]oxa-zol-2(3H)-one (0.050 g, 0.117 mmol) was obtained 0.005 g (10%) of the title compound as a white solid. ¹H NMR (DMSO-d₆): 7.93 (s, 1H), 7.87 (d, J=8.1 Hz, 1H), 7.64-7.61 (m, 2H), 7.26 (d, J=8.4 Hz, 1H), 7.17 (d, J=9.2 Hz, 1H), 4.42 (s, 2H), 3.85 (s, 3H), 3.83 (s, 3H).

### EXAMPLE 36

### 3-(2-Methoxyphenyl)-6-(4-methoxyphenyl)-[1,2,4]triazolo[4,3-b][1,2,4]triazin-7(8H)-one

(a) 6-(4-Methoxyphenyl)-3-(methylthio)-1,2,4-triazin-5(2H)-one. To an oven-dried round bottom flask charged with a magnetic stir bar at rt under argon with 3,4-dihydro-6-(4-methoxyphenyl)-3-thioxo-1,2,4-triazin-5(2H)-one (2.00 g, 8.50 mmol) and 1N NaOH (8.5 mL) at 0° C. was added iodomethane (0.69 mL, 11 mmol) dropwise over 2 minutes. The resulting suspension was stirred at 0° C. and then equilibrated to rt. The suspension was diluted with water and the precipitate was filtered and collected to give 2.11 g (99%) of the title compound as a yellow solid. ¹H NMR (DMSO-d₆): 14.06 (br s, 1H), 8.06 (d, J=9.2 Hz, 2H), 7.01 (d, J=9.2 Hz, 2H), 3.81 (s, 3H), 2.53 (s, 3H).

(b) 3-Hydrazinyl-6-(4-methoxyphenyl)-1,2,4-triazin-5(2H)-one. To an oven-dried round bottom flask charged with a magnetic stir bar at rt under argon with 6-(4-methoxyphenyl)-3-(methylthio)-1,2,4-triazin-5(2H)-one (1.87 g, 7.50 mmol) and ethanol (16.0 mL) was added hydrazine hydrate (4.0 mL, 82 mmol). The resulting suspension was heated to 90° C. for 2.5 h and then cooled to rt. The reaction solution was stirred in an ice bath and the resulting precipitate was filtered and collected to give 1.09 g (62%) of the title compound as a white solid. ¹H NMR (DMSO-d₆): 8.63 (br s, 1H), 7.99 (d, J=8.7 Hz, 2H), 6.96 (d, J=9.0 Hz, 2H), 4.59 (br s, 2H), 3.79 (s, 3H).

(c) 3-(2-Methoxyphenyl)-6-(4-methoxyphenyl)-[1,2,4]triazolo[4,3-b][1,2,4]triazin-7(8H)-one. To an oven-dried round bottom flask charged with a magnetic stir bar at rt under argon with 3-hydrazinyl-6-(4-methoxyphenyl)-1,2,4-triazin-5(2H)-one (0.100 g, 0.429 mmol) and anhydrous pyridine (2.0 ml) at 0° C. was added in one portion 2-methoxybenzoyl chloride (0.110 g, 0.644 mmol). The yellow suspension was heated to reflux for 3 h and then cooled to rt. The resulting suspension was diluted with ethanol and the precipitate was removed by filtering through a Buchner funnel. The filtrate was collected and concentrated by rotary evaporation. The resulting residue was diluted with EtOAc (50 mL), washed with 1M HCl (15 mL), H₂O (20 mL), brine (10 mL), dried over MgSO₄, filtered and concentrated to a yellow solid. Purification by flash chromatography (silica gel, gradient elution with 1:1, Hexanes:EtOAc to iPrOH:CH₂Cl₂, 4:96) gave 0.006 g (4%) as a white solid. ¹H NMR (DMSO-d₆): 13.99 (br s, 1H), 7.96 (d, J=8.8 Hz, 1H), 7.65-7.55 (m, 2H), 7.27 (d, J=8.4 Hz, 1H), 7.14 (t, J=7.5 Hz, 1H), 7.02 (d, J=8.8 Hz, 2H), 3.84 (s, 3H), 3.80 (s, 3H).

### EXAMPLE 37

### 3-(3,4,5-Trimethoxyphenyl)-6-(4-methylphenyl)-1H-[1,2,4]triazolo[4,3-b][1,2,4]triazin-7-one

(a) 6-(4-Methylphenyl)-3-(methylthio)-2H-[1,2,4]triazin-5-one. The title compound was prepared in a manner similar to example 36a. From 6-(4-methylphenyl)-3-thioxo-3,4-dihydro-2H-[1,2,4]triazin-5-one (2.00 g, 9.12 mmol) and iodomethane (0.749 mL, 11.9 mmol) was obtained 1.10 g (52%) of the title compound as a yellow solid. ¹H NMR (DMSO-d₆): 14.07 (br s, 1H), 7.95 (d, J=8.4 Hz, 2H), 7.26 (d, J=8.1 Hz, 2H), 2.53 (s, 3H), 2.35 (s, 3H).

(b) 3-Hydrazino-6-(4-methylphenyl)-2H-[1,2,4]triazin-5-one. The title compound was prepared in a manner similar to example 36b. From 6-(4-methylphenyl)-3-(methylthio)-2H-[1,2,4]triazin-5-one (1.10 g, 4.78 mmol) and hydrazine hydrate (2.4 mL, 49 mmol) was obtained 0.793 g (76%) of the title compound as a white solid. ¹H NMR (DMSO-d₆): 7.89 (d, J=8.1 Hz, 2H), 7.21 (d, J=8.1 Hz, 2H), 2.33 (s, 3H).

(c) 3-(3,4,5-Trimethoxyphenyl)-6-(4-methylphenyl)-1H-[1,2,4]triazolo[4,3-b][1,2,4]triazin-7-one. The title compound was prepared in a manner similar to example 36c. From 3-hydrazino-6-(4-methylphenyl)-2H-[1,2,4]triazin-5-one (0.093 g, 0.43 mmol) and 3,4,5-trimethoxybenzoyl chloride (0.149 g, 0.644 mmol) was obtained 0.006 g (4%) of the title compound as a yellow solid. ¹H NMR (DMSO-d₆): 8.12 (d, J=8.1 Hz, 2H), 7.66 (s, 2H), 7.26 (d, J=7.3 Hz, 2H), 3.87 (s, 6H), 3.72 (s, 3H), 2.36 (s, 3H).

### EXAMPLE 38

### 3-(2-Methoxyphenyl)-6-(4-methylphenyl)-1H-[1,2,4]triazolo[4,3-b][1,2,4]triazin-7-one

(a) 6-(4-Methylphenyl)-3-(methylthio)-2H-[1,2,4]triazin-5-one. The title compound was prepared in a manner similar to example 36a. From 6-(4-methylphenyl)-3-thioxo-3,4-dihydro-2H-[1,2,4]triazin-5-one (2.00 g, 9.12 mmol) and iodomethane (0.749 mL, 11.9 mmol) was obtained 1.10 g (52%) of the title compound as a yellow solid. ¹H NMR DMSO-d₆): 14.07 (br s, 1H), 7.95 (d, J=8.4 Hz, 2H), 7.26 (d, J=8.1 Hz, 2H), 2.53 (s, 3H), 2.35 (s, 3H).

(b) 3-Hydrazino-6-(4-methylphenyl)-2H-[1,2,4]triazin-5-one. The title compound was prepared in a manner similar to example 36b. From 6-(4-methylphenyl)-3-(methylthio)-2H-[1,2,4]triazin-5-one (1.10 g, 4.78 mmol) and hydrazine hydrate (2.4 mL, 49 mmol) was obtained 0.793 g (76%) of the title compound as a white solid. ¹H NMR (DMSO-d₆): 7.89 (d, J=8.1 Hz, 2H), 7.21 (d, J=8.1 Hz, 2H), 2.33 (s, 3H).

(c) 3-(2-methoxyphenyl)-6-(4-methylphenyl)-1H-[1,2,4]triazolo[4,3-b][1,2,4]triazin-7-one. The title compound was prepared in a manner similar to example 36c. From 3-hydrazino-6-(4-methylphenyl)-2H-[1,2,4]triazin-5-one (0.093 g, 0.43 mmol) and 2-methoxybenzoyl chloride (0.110 g, 0.644 mmol) was obtained 0.008 g (6%) of the title compound as a yellow solid. ¹H NMR (DMSO-d₆): 7.84 (d, J=8.1 Hz, 2H), 7.59-7.51 (m, 2H), 7.23 (d, J=8.4 Hz, 3H), 7.11 (t, J=7.0 Hz, 1H), 3.81 (s, 3H), 2.33 (s, 3H).

### EXAMPLE 39

### 3-(2-Methoxyphenyl)-6-(2,4-dimethylphenyl)-7H-[1,2,4]triazolo[3,4-b][1,3,4]thiadiazine

(a) 2-(3-(2-Methoxyphenyl)-1,2,4-triazole-4-amino-5-ylthio)-1-(2,4-dimethylphenyl)ethanone. The title compound was prepared in a manner similar to example 33a. From 2-bromo-1-(2,4-dimethylphenyl)ethanone (0.500 g, 2.20 mmol) and 4-amino-5-(2-methoxyphenyl)-3-mercapto-(4H)-1,2,4-triazole (0.489 g, 2.20 mmol) was obtained 0.747 g (92%) of the title compound as a white solid. ¹H NMR (DMSO-d₆): 7.91 (d, J=7.7 Hz, 1H), 7.66-7.58 (m, 2H), 7.27 (d, J=8.4 Hz, 1H), 7.21-7.13 (m, 3H), 4.91 (s, 2H), 3.86 (s, 3H), 2.42 (s, 3H), 2.35 (s, 3H).

(b) 3-(2-Methoxyphenyl)-6-(2,4-dimethylphenyl)-7H-[1,2,4]triazolo[3,4-b]-[1,3,4]thiadiazine. The title compound was prepared in a manner similar to example 33b. From 2-(3-(2-methoxyphenyl)-1,2,4-triazole-4-amino-5-ylthio)-1-(2,4-dimethylphenyl)ethanone (0.747 g, 2.03 mmol) was obtained 0.195 g (27%) of the title compound as a yellow solid. ¹H NMR (DMSO-d₆): 7.53 (td, J=7.8 and 1.3 Hz, 1H), 7.45 (dd, J=7.5 and 1.6 Hz, 1H), 7.35 (d, J=8.4 Hz, 1H), 7.16-7.07 (m, 4H), 4.42 (s, 2H), 3.73 (s, 3H), 2.29 (s, 3H), 2.27 (s, 3H).

### EXAMPLE 40

### 3-(2-Methoxyphenyl)-6-(3,4-dimethylphenyl)-7H-[1,2,4]triazolo[3,4-b][1,3,4]thiadiazine

To an oven-dried round bottom reaction flask charged with a magnetic stir bar under argon at rt with 2-bromo-1-(3,4-dimethylphenyl)ethanone (0.200 g, 0.881 mmol) and 4-amino-5-(2-methoxyphenyl)-3-mercapto-(4H)-1,2,4-triazole (0.196 g, 0.881 mmol) was added anhydrous isopropanol (4.4 mL). The resulting white suspension was refluxed at 130° C. for 14 h and then cooled to rt. The solvent was removed under rotary evaporation to leave a yellow solid. The yellow solid was diluted with EtOAc (75 mL), washed with NaHCO_{3(aq)} (2×25 mL) and brine (20 mL), dried over MgSO₄, filtered and concentrated to a brown residue. It was purified by flash column chromatography (silica gel, elution with EtOAc:hexanes, 1:1) to give 0.090 g (29%) of the title compound as a yellow solid. ¹H NMR (DMSO-d₆): 7.65 (s, 1H), 7.60-7.55 (m, 2H), 7.51 (dd, J=7.7 and 1.5 Hz, 1H), 7.28 (d, J=7.7 Hz, 1H), 7.23 (d, J=8.4 Hz, 1H), 7.12 (t, J=7.3 Hz, 1H), 4.40 (s, 2H), 3.74 (s, 3H), 2.27 (s, 3H), 2.26 (s, 3H).

### EXAMPLE 41

### 6-(2-Methoxy-4-methylphenyl)-3-(2-methoxyphenyl)-7H-[1,2,4]triazolo[3,4-b][1,3,4]thiadiazine

The title compound was prepared in a manner similar to example 40. From 2-bromo-1-(2-methoxy-4-methylphenyl)ethanone (0.214 g, 0.881 mmol) and 4-amino-5-(2-methoxyphenyl)-3-mercapto-(4H)-1,2,4-triazole (0.196 g, 0.881 mmol) was obtained 0.177 g (55%) of the title compound as a white solid. ¹H NMR (DMSO-d₆): 7.56-7.49 (m, 1H), 7.47 (dd, J=7.7 and 1.7 Hz, 1H), 7.25 (d, J=8.0 Hz, 1H), 7.17 (d, J=8.0 Hz, 1H), 7.08 (td, J=7.5 and 0.9 Hz, 1H), 7.03 (s, 1H), 6.83 (dd, J=7.7 and 0.5 Hz, 1H), 4.18 (s, 2H), 3.86 (s, 3H), 3.74 (s, 3H), 2.35 (s, 3H).

### EXAMPLE 42

### 6-(2,3,4-Trimethoxyphenyl)-3-(2-methoxyphenyl)-7H-[1,2,4]triazolo[3,4-b][1,3,4]thiadiazine

The title compound was prepared in a manner similar to example 40. From 2-bromo-1-(2,3,4-trimethoxyphenyl)ethanone (0.128 g, 0.441 mmol) and 4-amino-5-(2-methoxyphenyl)-3-mercapto-(4H)-1,2,4-triazole (0.098 g, 0.44 mmol) was obtained 0.018 g (10%) of the title compound as a white solid. ¹H NMR (CDCl₃): 7.63-7.61 (m, 1H), 7.50-7.45 (m, 1H), 7.22 (d, J=8.5 Hz, 1H), 7.08 (t, J=7.6 Hz, 1H), 6.99 (d, J=8.5 Hz, 1H), 6.69 (d, J=8.5 Hz, 1H), 3.97 (s, 2H), 3.94 (s, 3H), 3.89 (s, 6H), 3.79 (s, 3H).

### EXAMPLE 43

### 6-(2,3-Dihydrobenzo[b][1,4]dioxin-6-yl)-3-(2-methoxyphenyl)-7H-[1,2,4]triazolo[3,4-b][1,3,4]thiadiazine

The title compound was prepared in a manner similar to example 40. From 2-bromo-1-(2,3-dihydro[b][1,4]dioxin-6-yl)ethanone (0.113 g, 0.441 mmol) and 4-amino-5-(2-methoxyphenyl)-3-mercapto-(4H)-1,2,4-triazole (0.098 g, 0.44 mmol) was obtained 0.025 g (15%) of the title compound as a white solid. ¹H NMR (CDCl₃): 7.61 (dd, J=7.6 and 1.8 Hz, 1H), 7.50 (td, J=7.8 and 1.6 Hz, 1H), 7.34 (d, J=1.9 Hz, 1H), 7.30 (dd, J=8.5 and 2.2 Hz, 1H), 7.12-7.07 (m, 1H), 7.02 (d, J=8.2 Hz, 1H), 6.92 (d, J=8.5 Hz, 1H), 4.32-4.27 (m, 4H), 3.92 (s, 2H), 3.76 (s, 3H).

### EXAMPLE 44

### 6-(4-Isopropylphenyl)-3-(2-methoxyphenyl)-7H-[1,2,4]triazolo[3,4-b][1,3,4]thiadiazine

(a) 2-Bromo-1-(4-isopropylphenyl)ethanone. To an oven-dried round bottom reaction flask charged with a magnetic stir bar under argon at rt was added 4′-isopropylacetophenone (0.831 g, 5.12 mmol) and anhydrous CH₂Cl₂ (12.8 mL). The clear solution was cooled to 0° C. and then bromine (0.26 mL, 5.1 mmol) was added dropwise over 3 minutes forming a red solution. After 1 h of stirring at 0° C. the resulting red solution was concentrated by rotary evaporation to yield a yellow solid. It was purified by flash column chromatography (silica gel, elution with EtOAc:hexanes, 1:80), gave 0.112 g (9%) of the title compound as a white solid. ¹H NMR (CDCl₃): 7.93 (d, J=8.2 Hz, 2H), 7.34 (d, J=8.2 Hz, 2H), 4.44 (s, 2H), 2.98 (septet, J=6.9 Hz, 1H), 1.28 (d, J=6.9 Hz, 6H).

(b) 6-(4-isopropylphenyl)-3-(2-methoxyphenyl)-7H-[1,2,4]triazolo[3,4-b][1,3,4]thiadiazine. The title compound was prepared in a manner similar to example 40. From 2-bromo-1-(4-isopropylphenyl)ethanone (0.112 g, 0.464 mmol) and 4-amino-5-(2-methoxyphenyl)-3-mercapto-(4H)-1,2,4-triazole (0.103 g, 0.464 mmol) was obtained 0.040 g (23%) of the title compound as a white solid. ¹H NMR (DMSO-d₆): 7.78 (d, J=8.2 Hz, 2H), 7.60-7.54 (m, 1H), 7.49 (dd, J=7.4 and 1.6 Hz, 1H), 7.39 (d, J=8.5 Hz, 2H), 7.22 (d, J=8.2 Hz, 1H), 7.11 (t, J=7.4 Hz, 1H), 4.41 (s, 2H), 3.74 (s, 3H), 2.94 (septet, J=6.9 Hz, 1H), 1.20 (d, J=6.9 Hz, 6H).

### EXAMPLE 45

### 6-(3-Methoxy-4-nitrophenyl)-3-(2-methoxyphenyl)-7H-[1,2,4]triazolo[3,4-b][1,3,4]thiadiazine

(a) 2-Bromo-1-(3-methoxy-4-nitrophenyl)ethanone. The title compound was prepared in a manner similar to example 44a. From 1-(3-methoxy-4-nitrophenyl)-1-ethanone (1.00 g, 5.12 mmol) and bromine (0.26 mL, 5.1 mmol) was obtained 0.661 g (47%) of the title compound as a white solid. ¹H NMR (CDCl₃): 7.89 (d, J=8.2 Hz, 1H), 7.72 (d, J=1.6 Hz, 1H), 7.61 (dd, J=8.5 and 1.6 Hz, 1H), 4.43 (s, 2H), 4.04 (s, 3H).

(b) 6-(3-Methoxy-4-nitrophenyl)-3-(2-methoxyphenyl)-7H-[1,2,4]triazolo[3,4-b]-[1,3,4]thiadiazine. To an oven-dried round bottom reaction flask charged with a magnetic stir bar under argon at rt with 2-bromo-1-(3-methoxy-4-nitrophenyl)ethanone (0.241 g, 0.881 mmol) and 4-amino-5-(2-methoxyphenyl)-3-mercapto-(4H)-1,2,4-triazole (0.196 g, 0.881 mmol) was added anhydrous isopropanol (4.4 mL). The resulting yellow solution was refluxed at 130° C. for 6 h and then cooled to rt. The resulting precipitate was filtered and collected on a Buchner funnel to give 0.355 g (84%) of the title compound as a yellow solid. ¹H NMR (DMSO-d₆): 8.02 (d, J=8.5 Hz, 1H), 7.74 (d, J=1.7 Hz, 1H), 7.62-7.52 (m, 3H), 7.23 (d, J=8.5 Hz, 1H), 7.12 (t, J=7.4 Hz, 1H), 4.51 (s, 2H), 3.96 (s, 3H), 3.76 (s, 3H).

### EXAMPLE 46

### 3-(2-Methoxyphenyl)-6-(4-methoxyphenyl)-7H-[1,2,4]triazolo[3,4-b][1,3,4]thiadiazine

The title compound was prepared in a manner similar to example 45b. From 4-methoxyphenacyl bromide (0.202 g, 0.881 mmol) and 4-amino-5-(2-methoxyphenyl)-3-mercapto-(4H)-1,2,4-triazole (0.196 g, 0.881 mmol) was obtained 0.234 g (61%) of the title compound as a white solid. ¹H NMR (DMSO-d₆): 7.85 (dd, J=7.0 and 2.1 Hz, 2H), 7.63-7.57 (m, 1H), 7.54 (dd, J=7.6 and 1.8 Hz, 1H), 7.24 (d, J=8.2 Hz, 1H), 7.14 (td, J=7.5 and 0.9 Hz, 1H), 7.08 (dd, J=7.0 and 2.1 Hz, 2H), 4.43 (s, 2H), 3.82 (s, 3H), 3.76 (s, 3H).

### EXAMPLE 47

### 6-(2,3-Dihydro-2-oxobenzo[d]oxazol-6-yl)-3-(2-methoxyphenyl)-7H-[1,2,4]triazolo[3,4-b][1,3,4]thiadiazine

The title compound was prepared in a manner similar to example 45b. From 6-(2-chloroacetyl)benzo[d]oxazol-2(3H)-one (0.093 g, 0.44 mmol) and 4-amino-5-(2-methoxyphenyl)-3-mercapto-(4H)-1,2,4-triazole (0.098 g, 0.44 mmol) was obtained 0.158 g (78%) of the title compound as a white solid. ¹H NMR (DMSO-d₆): 12.1 (br s, 1H), 7.76 (d, J=1.5 Hz, 1H), 7.71 (dd, J=8.2 and 1.7 Hz, 1H), 7.62-7.56 (m, 1H), 7.52 (dd, J=7.7 and 1.8 Hz, 1H), 7.23 (dd, J=8.2 and 1.7 Hz, 2H), 7.13 (td, J=7.5 and 1.0 Hz, 1H), 4.43 (s, 2H), 3.75 (s, 3H).

### EXAMPLE 48

### 6-(4-(Trifluoromethyl)phenyl)-3-(2-methoxyphenyl)-7H-[1,2,4]triazolo[3,4-b][1,3,4]thiadiazine

The title compound was prepared in a manner similar to example 45b. From 4-trifluoromethylphenacyl bromide (0.118 g, 0.441 mmol) and 4-amino-5-(2-methoxyphenyl)-3-mercapto-(4H)-1,2,4-triazole (0.098 g, 0.44 mmol) was obtained 0.091 g (44%) of the title compound as a yellow solid. ¹H NMR (DMSO-d₆): 8.06 (d, J=8.2 Hz, 2H), 7.91 (d, J=8.2 Hz, 2H), 7.62-7.56 (m, 1H), 7.51 (dd, J=7.4 and 1.7 Hz, 1H), 7.22 (d, J=8.0 Hz, 1H), 7.12 (td, J=7.5 and 0.9 Hz, 1H), 4.50 (s, 2H), 3.75 (s, 3H).

### EXAMPLE 49

### 6-(5-Chloro-2-methoxy-4-methylphenyl)-3-(2-methoxyphenyl)-7H-[1,2,4]triazolo[3,4-b][1,3,4]thiadiazine

The title compound was prepared in a manner similar to example 45b. From 2-bromo-1-(5-chloro-2-methoxy-4-methylphenyl)ethanone (0.122 g, 0.441 mmol) and 4-amino-5-(2-methoxyphenyl)-3-mercapto-(4H)-1,2,4-triazole (0.098 g, 0.44 mmol) was obtained 0.121 g (57%) of the title compound as a yellow solid. ¹H NMR (DMSO-d₆): 7.57-7.51 (m, 1H), 7.48 (dd, J=7.3 and 1.8 Hz, 1H), 7.35 (s, 1H), 7.25 (br s, 1H), 7.19 (d, J=8.4 Hz, 1H), 7.09 (td, J=7.5 and 0.9 Hz, 1H), 4.21 (s, 2H), 3.88 (s, 3H), 3.76 (s, 3H), 2.37 (s, 3H).

### EXAMPLE 50

### 3-(2-Methoxyphenyl)-6-(4-(trifluoromethoxy)phenyl)-7H-[1,2,4]triazolo[3,4-b][1,3,4]thiadiazine

The title compound was prepared in a manner similar to example 45b. From 4-(trifluoromethoxy)phenacyl bromide (0.125 g, 0.441 mmol) and 4-amino-5-(2-methoxyphenyl)-3-mercapto-(4H)-1,2,4-triazole (0.098 g, 0.44 mmol) was obtained 0.106 g (49%) of the title compound as a white solid. ¹H NMR (DMSO-d₆): 7.98 (dd, J=6.9 and 2.2 Hz, 2H), 7.61-7.49 (m, 4H), 7.22 (d, J=8.5 Hz, 1H), 7.12 (td, J=7.5 and 0.9 Hz, 1H), 4.46 (s, 2H), 3.75 (s, 3H).

### EXAMPLE 51

### 6-(4-tert-Butylphenyl)-3-(2-methylphenyl)-7H-[1,2,4]triazolo[3,4-b][1,3,4]thiadiazine

The title compound was prepared in a manner similar to example 45b. From 2-bromo-1-(4-tert-butylphenyl)ethanone (0.123 g, 0.485 mmol) and 4-amino-5-(2-methylphenyl)-3-mercapto-(4H)-1,2,4-triazole (0.100 g, 0.485 mmol) was obtained 0.122 g (57%) of the title compound as a white solid. ¹H NMR (DMSO-d₆): 7.80 (d, J=8.3 Hz, 2H), 7.54 (d, J=8.5 Hz, 2H), 7.51-7.34 (m, 4H), 4.45 (s, 2H), 2.36 (s, 3H), 1.29 (s, 9H).

### EXAMPLE 52

### 6-(4-(N,N-Diethylaminosulfonyl)phenyl)-3-(2-methylphenyl)-7H-[1,2,4]triazolo[3,4-b][1,3,4]thiadiazine

The title compound was prepared in a manner similar to example 45b. From 4-(bromoacetyl)-N,N-diethyl benzenesulfonamide (0.162 g, 0.485 mmol) and 4-amino-5-(2-methylphenyl)-3-mercapto-(4H)-1,2,4-triazole (0.100 g, 0.485 mmol) was obtained 0.184 g (73%) of the title compound as a yellow solid. ¹H NMR (DMSO-d₆): 7.15 (d, J=8.8 Hz, 2H), 7.93 (d, J=8.8 Hz, 2H), 7.52-7.34 (m, 4H), 4.51 (s, 2H), 3.18 (q, J=7.1 Hz, 4H), 2.37 (s, 3H), 1.04 (t, J=7.1 Hz, 6H).

### EXAMPLE 53

### 6-(4-Methyl-3-nitrophenyl)-3-(2-methylphenyl)-7H-[1,2,4]triazolo[3,4-b][1,3,4]thiadiazine

The title compound was prepared in a manner similar to example 45b. From 2-bromo-1-(4-methyl-3-nitrophenyl)ethanone (0.250 g, 0.970 mmol) and 4-amino-5-(2-methylphenyl)-3-mercapto-(4H)-1,2,4-triazole (0.200 g, 0.970 mmol) was obtained 0.429 g (99%) of the title compound as a white solid. ¹H NMR (DMSO-d₆): 8.44 (d, J=1.4 Hz, 1H), 8.07 (dd, J=8.1 and 1.8 Hz, 1H), 7.68 (d, J=8.2 Hz, 1H), 7.52-7.34 (m, 4H), 4.51 (s, 2H), 2.57 (s, 3H), 2.37 (s, 3H).

### EXAMPLE 54

### 3-(2-Methoxyphenyl)-6-(4-methyl-3-nitrophenyl)-7H-[1,2,4]triazolo[3,4-b][1,3,4]thiadiazine

The title compound was prepared in a manner similar to example 45b. From 2-bromo-1-(4-methyl-3-nitrophenyl)ethanone (0.250 g, 0.970 mmol) and 4-amino-5-(2-methoxyphenyl)-3-mercapto-(4H)-1,2,4-triazole (0.215 g, 0.970 mmol) was obtained 0.395 g (88%) of the title compound as a white solid. ¹H NMR (DMSO-d₆): 8.44 (d, J=1.7 Hz, 1H), 8.07 (dd, J=8.1 and 2.1 Hz, 1H), 7.68 (d, J=8.2 Hz, 1H), 7.62-7.56 (m, 1H), 7.52 (dd, J=7.7 and 1.7 Hz, 1H), 7.24 (d, J=7.7 Hz, 1H), 7.12 (td, J=7.4 and 0.8 Hz, 1H), 4.49 (s, 2H), 3.76 (s, 3H), 2.57 (s, 3H).

### EXAMPLE 55

### 6-(4-Chloro-3-nitrophenyl)-3-(2-methoxyphenyl)-7H-[1,2,4]triazolo[3,4-b][1,3,4]thiadiazine

The title compound was prepared in a manner similar to example 45b. From 2-bromo-1-(4-chloro-3-nitrophenyl)ethanone (0.270 g, 0.970 mmol) and 4-amino-5-(2-methoxyphenyl)-3-mercapto-(4H)-1,2,4-triazole (0.215 g, 0.970 mmol) was obtained 0.383 g (82%) of the title compound as a white solid. ¹H NMR (DMSO-d₆): 8.53 (d, J=2.2 Hz, 1H), 8.11 (dd, J=8.5 and 2.2 Hz, 1H), 7.96 (d, J=8.5 Hz, 1H), 7.62-7.56 (m, 1H), 7.52 (dd, J=7.6 and 1.8 Hz, 1H), 7.23 (d, J=7.7 Hz, 1H), 7.12 (td, J=7.5 and 0.9 Hz, 1H), 4.49 (s, 2H), 3.75 (s, 3H).

### EXAMPLE 56

### 6-(4-Acetamido-3-nitrophenyl)-3-(2-methoxyphenyl)-7H-[1,2,4]triazolo[3,4-b][1,3,4]thiadiazine

The title compound was prepared in a manner similar to example 45b. From 4-(chloroacetyl)-2-nitro-acetanilide (0.249 g, 0.970 mmol) and 4-amino-5-(2-methoxyphenyl)-3-mercapto-(4H)-1,2,4-triazole (0.215 g, 0.970 mmol) was obtained 0.414 g (93%) of the title compound as a brown solid. ¹H NMR (DMSO-d₆): 10.60 (br s, 1H), 8.39 (d, J=2.2 Hz, 1H), 8.12 (dd, J=8.5 and 2.2 Hz, 1H), 7.80 (d, J=8.5 Hz, 1H), 7.59 (td, J=7.9 and 1.5 Hz, 1H), 7.52 (dd, J=7.6 and 1.8 Hz, 1H), 7.23 (d, J=8.2 Hz, 1H), 7.12 (t, J=7.1 Hz, 1H), 4.47 (s, 2H), 3.76 (s, 3H), 2.10 (s, 3H).

### EXAMPLE 57

### 6-(4-Amino-3-methoxyphenyl)-3-(2-methoxyphenyl)-7H-[1,2,4]triazolo[3,4-b][1,3,4]thiadiazine

To a hydrogenation reaction flask was added 6-(3-methoxy-4-nitrophenyl)-3-(2-methoxyphenyl)-7H-[1,2,4]triazolo[3,4-b][1,3,4]thiadiazine (0.014 g, 0.035 mmol), EtOH (0.35 mL), ethyl acetate (0.35 mL) and 5% Pd/C (0.014 g). While being shaken at rt the reaction flask was degassed three times and filled with H_{2(g)} (45 psi). The reaction mixture was shaken under H_{2(g)} for 3 h and then the mixture was diluted with methanol and filtered through celite. The organic filtrate was collected and concentrated to a green solid. It was purified by flash column chromatography (silica gel, elution with EtOAc) to give 0.006 g (46%) of the title compound as a white solid. ¹H NMR (CDCl₃): 7.64 (dd, J=7.6 and 1.8 Hz, 1H), 7.51-7.45 (m, 1H), 7.33 (d, J=1.9 Hz, 1H), 7.22 (dd, J=8.2 and 1.9 Hz, 1H), 7.08 (td, J=7.6 and 1.0 Hz, 1H), 7.02-6.99 (m, 1H), 6.69 (d, J=8.2 Hz, 1H), 3.93 (s, 2H), 3.82 (s, 3H), 3.76 (s, 3H).

### EXAMPLE 58

### 6-(3-Amino-4-methylphenyl)-3-(2-methoxyphenyl)-7H-[1,2,4]triazolo[3,4-b][1,3,4]thiadiazine

To an oven-dried round bottom reaction flask charged with a magnetic stir bar under argon at rt with 3-(2-methoxyphenyl)-6-(4-methyl-3-nitrophenyl)-7H-[1,2,4]triazolo[3,4-b][1,3,4]thiadiazine (0.100 g, 0.262 mmol) and tin (II) chloride dihydrate (0.266 g, 1.18 mmol) was added anhydrous ethanol (1.3 mL). The resulting white suspension was heated at 90° C. for 7 h and then cooled to rt. The solvent was removed by rotary evaporation, diluted with H₂O (70 mL) and then basified using 2N NaOH until the pH=9. The aqueous layer was then extracted with EtOAc (2×125 mL), washed with brine (30 mL), dried over MgSO₄, filtered and concentrated to give 0.035 g (38%) of the title compound as a brown solid. ¹H NMR (CD₃OD): 7.60-7.53 (m, 2H), 7.30 (d, J=1.4 Hz, 1H), 7.25-7.10 (m, 4H), 4.24 (s, 2H), 3.77 (s, 3H), 2.22 (s, 3H).

### EXAMPLE 59

### 6-(3-Amino-4-chlorophenyl)-3-(2-methoxyphenyl)-7H-[1,2,4]triazolo[3,4-b][1,3,4]thiadiazine

The title compound was prepared in a manner similar to example 58. From 6-(4-chloro-3-nitrophenyl)-3-(2-methoxyphenyl)-7H-[1,2,4]triazolo[3,4-b][1,3,4]thiadiazine (0.075 g, 0.187 mmol) and tin (II) chloride dihydrate (0.189 g, 0.840 mmol) was obtained 0.025 g (36%) of the title compound as a yellow solid. ¹H NMR (DMSO-d₆): 7.60-7.55 (m, 1H), 7.49 (dd, J=7.6 and 1.8 Hz, 1H), 7.34 (d, J=8.5 Hz, 1H), 7.22-7.19 (m, 2H), 7.14-7.09 (m, 1H), 7.03 (dd, J=8.5 and 2.2 Hz, 1H), 5.66 (br s, 2H), 4.33 (s, 2H), 3.74 (s, 3H).

### EXAMPLE 60

### 6-(3,4-Diaminophenyl)-3-(2-methoxyphenyl)-7H-[1,2,4]triazolo[3,4-b][1,3,4]thiadiazine

The title compound was prepared in a manner similar to example 58. From 6-(4-acetamido-3-nitrophenyl)-3-(2-methoxyphenyl)-7H-[1,2,4]triazolo[3,4-b][1,3,4]thiadiazine (0.250 g, 0.590 mmol) and tin (II) chloride dihydrate (0.598 g, 2.65 mmol) was obtained the crude product. It was purified by flash column chromatography (silica gel, elution with EtOAc) to give 0.025 g (10%) of the title compound as a yellow solid. ¹H NMR (CD₃OD): 7.59-7.56 (m, 1H), 7.53 (dd, J=7.3 and 1.8 Hz, 1H), 7.27 (d, J=2.2 Hz, 1H), 7.19 (dd, J=8.2 and 2.2 Hz, 1H), 7.17 (d, J=8.2 Hz, 1H), 7.12 (td, J=7.6 and 1.0 Hz, 1H), 6.68 (d, J=8.2 Hz, 1H), 4.16 (s, 2H), 3.77 (s, 3H).

### EXAMPLE 61

### 6-(3-Amino-4-methylphenyl)-3-(2-methylphenyl)-7H-[1,2,4]triazolo[3,4-b][1,3,4]thiadiazine

The title compound was prepared in a manner similar to example 58. From 6-(4-methyl-3-nitrophenyl)-3-(2-methylphenyl)-7H-[1,2,4]triazolo[3,4-b][1,3,4]thiadiazine (0.120 g, 0.328 mmol) and tin (II) chloride dihydrate (0.333 g, 1.48 mmol) was obtained 0.030 g (27%) of the title compound as a yellow solid. ¹H NMR (DMSO-d₆): 7.50-7.33 (m, 4H), 7.08-7.05 (m, 2H), 6.99 (dd, J=7.7 and 1.6 Hz, 1H), 5.13 (br s, 2H), 4.34 (s, 2H), 2.35 (s, 3H), 2.09 (s, 3H).

### EXAMPLE 62

### 3-(5-Chloro-2-methoxyphenyl)-6-(4-methylphenyl)-7H-[1,2,4]triazolo[3,4-b][1,3,4]thiadiazine

The title compound was prepared in a manner similar to example 2. From 2-bromo-1-(4-methylphenyl)ethanone (426 mg, 2 mmol) and 4-amino-5-(5-chloro-2-methoxyphenyl)-3-mercapto-(4H)-1,2,4-triazole (472 mg, 2 mmol) was obtained 252 mg (34%) of the title compound as solids. ¹H NMR (CDCl₃): 7.70 (d, J=8.4 Hz, 2H), 7.62 (d, J=2.7 Hz, 1H), 7.62 (dd, J1=8.7 Hz, J2=2.7 Hz, 1H), 7.26 (d, J=8.7 Hz, 2H), 6.95 (d, J=9.0 Hz, 1H), 3.98 (s, 2H), 3.73 (s, 3H), 2.42 (s, 3H).

### EXAMPLE 63

### 3-(2-Methoxyphenyl)-6-(3-methoxylphenyl)-7H-[1,2,4]triazolo[3,4-b][1,3,4]thiadiazine

The title compound was prepared in a manner similar to example 2. From 2-bromo-1-(3-methoxyphenyl)ethanone (229 mg, 1 mmol) and 4-amino-5-(2-methoxyphenyl)-3-mercapto-(4H)-1,2,4-triazole (222 mg, 1 mmol) was obtained 236 mg (67%) of the title compound as solids. ¹H NMR (CDCl₃): 7.62 (dd, J1=7.2 Hz, J2=1.5 Hz, 1H), 7.50 (dt, J1=8.4 Hz, J2=1.5 Hz, 1H), 7.41-7.32 (m, 4H), 7.12-7.00 (m, 3H), 3.98 (s, 2H), 3.81 (s, 3H), 3.76 (s, 3H).

### EXAMPLE 64

### 3-(2-Methoxyphenyl)-6-(2-methoxylphenyl)-7H-[1,2,4]triazolo[3,4-b][1,3,4]thiadiazine

The title compound was prepared in a manner similar to example 2. From 2-bromo-1-(2-methoxyphenyl)ethanone (229 mg, 1 mmol) and 4-amino-5-(2-methoxyphenyl)-3-mercapto-(4H)-1,2,4-triazole (222 mg, 1 mmol) was obtained 314 mg (89%) of the title compound as solids. ¹H NMR (CDCl₃): 7.61 (dd, J1=7.5 Hz, J2=1.8 Hz, 1H), 7.55-7.42 (m, 3H), 7.08-7.03 (t, J=7.5 Hz, 1H), 7.02-6.96 (m, 3H), 3.95 (s, 2H), 3.92 (s, 3H), 3.78 (s, 3H).

### EXAMPLE 65

### 3-(2,3-Dimethoxyphenyl)-6-(4-methylphenyl)-7H-[1,2,4]triazolo[3,4-b][1,3,4]thiadiazine

(a) 2,3-dimethoxybenzoyl-2-dithiocarboxyhydrazide, potassium salt. To a solution of 2,3-dimethoxybenzohydrazide (1.5 g, 7.64 mmol) in ethanol (60 mL) was added potassium hydroxide (0.76 g, 11.46 mmol, 85% pure), followed by carbon disulfide (0.87 g, 11.46 mmol). The solution was stirred at rt for 3 h and then ethyl ether (100 mL) was added. The precipitates were collected by filtration and washed with ether three times, and dried to give the title compounds as solids.

(b) 4-amino-5-(2,3-dimethoxyphenyl)-3-mercapto-(4H)-1,2,4-triazole. A mixture of the above solids with hydrazine hydrate (0.39 g, 12.24 mmol) in water (2 mL) was heated to reflux for 1 h until the color of the solution become clear green. After cooling to room temperature, 100 mL of ice water was added to the solution and it was neutralized with 3N hydrochloric acid to form a precipitate, which was collected through filtration. The solids was purified by recrystallization from ethanol to give 305 mg (20%) of the title compound. ¹H NMR (DMSO-d₆): 7.29-7.25 (m, 1H), 7.212-7.16 (m, 1H), 7.03-7.00 (m, 1H), 5.49 (s, 2H), 3.87 (s, 3H), 3.72 (s, 3H).

(c) 3-(2,3-dimethoxyphenyl)-6-(4-methylphenyl)-7H-[1,2,4]triazolo[3,4-b][1,3,4]thiadiazines. The title compound was prepared in a manner similar to example 10. From 2-bromo-1-(4-methylphenyl)ethanone (169 mg, 0.79 mmol) and 4-amino-5-(2,3-dimethoxyphenyl)-3-mercapto-(4H)-1,2,4-triazole (200 mg, 0.79 mmol) was obtained 301 mg (95%) of the title compound as solids. ¹H NMR (CD₃OD): 8.88 (d, J=8.4 Hz, 2H), 7.45-7.30 (m, 5H), 4.45 (s, 2H), 4.03 (s, 3H), 3.96 (s, 3H), 3.85 (s, 3H), 2.43 (s, 3H).

### EXAMPLE 66

### 6-(4-Aminophenyl)-3-(3,4,5-trimethoxyphenyl)-7H-[1,2,4]triazolo[3,4-b][1,3,4]thiadiazine

A mixture of 3-(3,4,5-trimethoxyphenyl)-6-(4-nitrophenyl)-7H-[1,2,4]triazolo[3,4-b][1,3,4]thiadiazine (600 mg, 1.41 mmol), concentrated hydrochloride (0.5 mL) and 5% palladium on carbon (150 mg) in ethanol (100 mL) and THF (50 mL) was hydrogenated under 48 psi of hydrogen overnight. It was filtered and neutralized with aqueous sodium carbonate. The mixture was evaporated and washed with water (50 mL), the solid was collected through filtration and purified by column chromatography (EtOAc/CH₂Cl₂ 1:1) to give 105 mg (19%) of the title compound as solids. ¹H NMR (CDCl₃): 7.79 (d, J=8.7 Hz, 2H), 7.52 (s, 2H), 6.73 (d, J=8.7 Hz, 2H), 4.20 (s, 2H), 3.94 (s, 2H), 3.93-3.91 (s, 9H).

### EXAMPLE 67

### 6-(4-Dimethylaminophenyl)-3-(3,4,5-trimethoxyphenyl)-7H-[1,2,4]triazolo[3,4-b][1,3,4]thiadiazine

To a mixture of 6-(4-aminophenyl)-3-(3,4,5-trimethoxyphenyl)-7H-[1,2,4]triazolo[3,4-b][1,3,4]thiadiazine (75 mg, 0.19 mmol) in methanol (10 mL) was added 37% aqueous formaldehyde (56 mg, 1.86 mmol) and acetic acid (0.3 mL). It was stirred at rt for 1 h, then was cooled to 0° C. To the solution was added sodium cyanoborohydride (167 mg, 1.86 mmol) and it was stirred at rt for 2 h. It was evaporated and the residue was dissolved in ethyl acetate (30 mL). The solution was washed with water (30 mL), dried and evaporated and the residue was purified by column chromatography to give the solid, which was redissolved in methanol (8 mL) and acidified with 2 N hydrochloride in ether (1 mL), and the mixture was evaporated to give 50 mg (58%) of the title compound as solids. ¹H NMR (CD₃OD): 8.05 (d, J=9.0 Hz, 2H), 7.50 (s, 2H), 6.95 (d, J=9.0 Hz, 2H), 4.45 (s, 2H), 3.93 (s, 6H), 3.92 (s, 3H), 3.13 (s, 6H).

### EXAMPLE 68

### 3-(2-Fluorophenyl)-6-(4-methylphenyl)-7H-[1,2,4]triazolo[3,4-b][1,3,4]thiadiazine

The title compound was prepared in a manner similar to example 6. From 2-bromo-1-(4-methylphenyl)ethanone (319 mg, 1.5 mmol) and 4-amino-5-(2-fluorophenyl)-3-mercapto-(4H)-1,2,4-triazole (315 mg, 1.5 mmol) was obtained 0.42 g (70%) of the title compound as solids. ¹H NMR (CD₃OD): 7.96 (d, J=8.4 Hz, 2H), 7.94-7.91 (m, 1H), 7.88-7.84 (m, 1H), 7.72-7.65 (m, 1H), 7.48-7.40 (m, 1H), 7.41 (d, J=9.0 Hz, 2H), 4.47 (s, 2H), 2.45 (s, 3H).

### EXAMPLE 69

### 3-(2,5-Dimethoxyphenyl)-6-(4-methylphenyl)-7H-[1,2,4]triazolo[3,4-b][1,3,4]thiadiazine

The title compound was prepared in a manner similar to example 2. From 2-bromo-1-(4-methylphenyl)ethanone (151 mg, 0.71 mmol) and 4-amino-5-(2,5-dimethoxyphenyl)-3-mercapto-(4H)-1,2,4-triazole (151 mg, 0.71 mmol) was obtained 21 mg (8%) of the title compound as solids. ¹H NMR (CD₃OD): 7.95 (d, J=8.1 Hz, 2H), 7.56 (d, J=2.7 Hz, 1H), 7.40 (d, J=8.1 Hz, 2H), 7.38-7.30 (m, 1H), 7.31 (s, 1H), 4.47 (s, 2H), 3.94 (s, 3H), 3.81 (s, 3H), 2.45 (s, 3H).

### EXAMPLE 70

### 3-(2-Methylphenyl)-6-(4-methylphenyl)-7H-[1,2,4]triazolo[3,4-b][1,3,4]thiadiazine

The title compound was prepared in a manner similar to example 6. From 2-bromo-1-(4-methylphenyl)ethanone (319 mg, 1.5 mmol) and 4-amino-5-(2-methylphenyl)-3-mercapto-(4H)-1,2,4-triazole (310 mg, 1.5 mmol) was obtained 0.41 g (85%) of the title compound as solids. ¹H NMR (CD₃OD): 7.87 (d, J=8.7 Hz, 2H), 7.52-7.38 (m, 4H), 7.34 (d, J=8.7 Hz, 2H), 4.45 (s, 2H), 2.51 (s, 3H), 2.36 (s, 3H).

### EXAMPLE 71

### 3-(2-Methylthiophenyl)-6-(4-methylphenyl)-7H-[1,2,4]triazolo[3,4-b][1,3,4]thiadiazine

The title compound was prepared in a manner similar to example 2. From 2-bromo-1-(4-methylphenyl)ethanone (476 mg, 2 mmol) and 4-amino-5-(2-meththiophenyl)-3-mercapto-(4H)-1,2,4-triazole (426 mg, 2 mmol) was obtained 405 mg (58%) of the title compound as solids. ¹H NMR (CDCl₃): 7.71 (d, J=8.7 Hz, 2H), 7.60 (d, J=6.9 Hz, 1H), 7.49-7.46 (m, 2H), 7.33-7.24 (m, 3H), 3.98 (s, 2H), 2.41 (s, 3H), 2.39 (s, 3H).

### EXAMPLE 72 AND 73

### 3-(2-Methylsulfinylphenyl)-6-(4-methylphenyl)-7H-[1,2,4]triazolo[3,4-b][1,3,4]thiadiazine and 3-(2-Methylsulfonylphenyl)-6-(4-methylphenyl)-7H-[1,2,4]triazolo[3,4-b][1,3,4]thiadiazine

To a 0° C. solution of 3-(2-meththiophenyl)-6-(4-methylphenyl)-7H-[1,2,4]triazolo[3,4-b][1,3,4]thiadiazine (105 mg, 0.297 mmol) in dichloromethane (2 mL) was added meta-chloroperbenzoic acid (mCPBA, 134 mg, 0.60 mmol), and it was warmed to rt and stirred for 1 h. It was diluted with dichloromethane, washed with aqueous Na₂S₂O₃ and washed with water. The organic layer was dried and concentrated to give the crude mixture, which was purified by column chromatography to give 3-(2-methsulfinylphenyl)-6-(4-methylphenyl)-7H-[1,2,4]triazolo[3,4-b][1,3,4]thiadiazine (16 mg, 15%). ¹H NMR (CDCl₃) 8.36 (d, J=8.1 Hz, 1H), 7.87 (d, J=7.8 Hz, 1H), 7.80-7.75 (m, 1H), 7.77-7.74 (d, J=8.4 Hz, 2H), 7.64-7.59 (dt, J₁=7.8, J₂=0.6 Hz, 1H), 7.32-7.29 (d, J=8.4 Hz, 2H), 4.10-3.95 (m, 2H), 3.05 (s, 3H), 2.44 (s, 3H); and 3-(2-methsulfonylphenyl)-6-(4-methylphenyl)-7H-[1,2,4]triazolo[3,4-b][1,3,4]thiadiazine (30 mg, 26%). ¹H NMR (CDCl₃) 8.24-8.21 (m, 1H), 7.82-7.67 (m, 3H), 7.61-7.59 (d, J=8.4 Hz, 2H), 7.23-7.20 (d, J=8.4 Hz, 2H), 3.98 (s, 2H), 3.29 (s, 3H), 2.38 (s, 3H).

### EXAMPLE 74

### 3-(2-Aminophenyl)-6-(4-methylphenyl)-7H-[1,2,4]triazolo[3,4-b][1,3,4]thiadiazine

The title compound was prepared in a manner similar to example 2. From 2-bromo-1-(4-methylphenyl)ethanone (162 mg, 0.782 mmol) and 4-amino-5-(2-aminophenyl)-3-mercapto-(4H)-1,2,4-triazole (167 mg, 0.782 mmol) was obtained a crude product which was purified by column chromatography (EtOAc/Hexane 2:1) to give 41 mg (16%) of the title compound. ¹H NMR (CDCl₃): 7.90-7.87 (dd, J1=8.1 Hz, J2=1.5 Hz, 1H), 7.82 (d, J=6.6 Hz, 2H), 7.33 (d, J=7.8 Hz, 2H), 7.25-7.20 (ddd, J1=8.4 Hz, J2=7.2 Hz, J3=1.5 Hz, 1H), 6.84-6.81 (dd, J1=8.1 Hz, J2=0.9 Hz, 1H), 7.25-7.20 (ddd, J1=8.4 Hz, J2=7.2 Hz, J3=1.5 Hz, 1H), 5.85 (bs, 2H), 3.97 (s, 2H), 2.45 (s, 3H).

### EXAMPLE 75

### 6-(4-Aminophenyl)-3-(2-methoxyphenyl)-7H-[1,2,4]triazolo[3,4-b][1,3,4]thiadiazine

A mixture of 3-(2-methoxyphenyl)-6-(4-nitrophenyl)-7H-[1,2,4]triazolo[3,4-b][1,3,4]thiadiazine (142 mg, 0.39 mmol) in THF (2 mL), methanol (25 mL) and HCl (1N, 2 mL) was hydrogenated overnight at 60 psi over Palladium on carbon (5% Pd content, 100 mg). The resulting mixture was filtered through a pad of Celite and the filtrate was concentrated under vacuum. The residue was treated with 50 mL of saturated NaHCO₃ and extracted with 50 mL ethyl acetate. The organic layer was dried over anhydrous Na₂SO₄, filtered and concentrated. The residue was purified by chromatography (90% ethyl acetate/hexane, 0.3 mL methanol/10 mL) to give the title compound (103 mg, 0.31 mmol, 79%) as solids. ¹H NMR (CDCl₃, MeOH-d₄): 7.59-7.66 (m, 2H), 7.56 (dd, 1H, J=1.8, 7.5 Hz), 7.52 (ddd, 1H, J=9.3, 7.8, 2.1 Hz), 7.10 (dt, 1H, J=7.5, 0.9 Hz), 7.03 (d, 1H, J=8.4 Hz), 6.64-6.71 (m, 2H), 3.96 (s, 2H), 3.75 (s, 3H).

### EXAMPLE 76

### 6-(4-Azidophenyl)-3-(2-methoxyphenyl)-7H-[1,2,4]triazolo[3,4-b][1,3,4]thiadiazine

The title compound was prepared in a manner similar to example 31 from 6-(4-aminophenyl)-3-(2-methoxyphenyl)-7H-[1,2,4]triazolo[3,4-b][1,3,4]thiadiazine and was isolated as solids. ¹H NMR (CDCl₃): 7.80 (m, 2H), 7.61 (dd, 1H, J=7.5, 1.8 Hz), 7.51 (ddd, 1H, J=8.4, 7.5, 1.5 Hz), 7.06-7.11 (m, 3H), 7.03 (d, 1H, J=8.4 Hz), 3.97 (s, 2H), 3.74 (s, 3H).

### EXAMPLE 77

### 6-(4-dimethylaminophenyl)-3-(2-methoxyphenyl)-7H-[1,2,4]triazolo[3,4-b][1,3,4]thiadiazine

The title compound was prepared in a manner similar to example 30 from 6-(4-aminophenyl)-3-(2-methoxyphenyl)-7H-[1,2,4]triazolo[3,4-b][1,3,4]thiadiazine and was isolated as solids. ¹H NMR (CDCl₃): 7.69 (m, 2H), 7.61 (dd, 1H, J=7.5, 1.8 Hz), 7.47 (ddd, 1H, J=8.4, 7.8, 2.1 Hz), 7.08 (dt, 1H, J=7.5, 0.9 Hz), 7.01 (d, 1H, J=8.4 Hz), 6.66 (m, 2H), 3.90 (s, 2H), 3.74 (s, 3H), 3.03 (s, 6H).

### EXAMPLE 78

### 6-(4-Amino-3,5-dibromophenyl)-3-(2-methoxyphenyl)-7H-[1,2,4]triazolo[3,4-b][1,3,4]thiadiazine

A solution of bromine (200 mg, 10.1 mmol) in THF (1 mL) and glacial acetic acid (1 mL) was added to a solution of 6-(4-aminophenyl)-3-(2-methoxyphenyl)-7H-[1,2,4]triazolo[3,4-b][1,3,4]thiadiazine (200 mg, 0.59 mmol) in THF (4 mL) and glacial acetic acid (1 mL) at 0° C. and the mixture was stirred at the same temperature for 1 h. The reaction mixture was evaporated to dryness and the residue was dissolved in 50 mL of ethyl acetate. The solution was washed with saturated NaHCO₃, water, 10% Na₂S₂O₃, water and saturated NaCl, dried over anhydrous Na₂SO₄, filtered and concentrated. The residue was purified by chromatography (65% ethyl acetate/hexane) to give the title compound (28 mg, 0.057 mmol, 10%). ¹H NMR (CDCl₃): 7.84 (s, 2H), 7.62 (dd, 1H, J=1.5, 7.5 Hz), 7.51 (ddd, 1H, J=1.8, 7.5, 8.4 Hz), 7.10 (dt, 1H, J=0.6, 7.2 Hz), 7.02 (d, 1H, J=8.1 Hz), 5.00 (s, br, 2H), 3.87 (s, 2H), 3.76 (s, 3H).

### EXAMPLE 79

### 6-(4-Amino-3,5-diiodophenyl)-3-(2-methoxyphenyl)-7H-[1,2,4]triazolo[3,4-b][1,3,4]thiadiazine

To a solution of 6-(4-aminophenyl)-3-(2-methoxyphenyl)-7H-[1,2,4]triazolo[3,4-b][1,3,4]thiadiazine (153 mg, 0.45 mmol) in glacial acetic acid at 10° C. was added iodine monochloride (380 mg, 2.3 mmol). The mixture was stirred at the same temperature for 0.5 h, more iodine monochloride (250 mg, 1.5 mmol) was added and stirred for 2 h at room temperature. The reaction mixture was evaporated to dryness and the residue was dissolved in 50 mL of ethyl acetate. The solution was washed with saturated NaCO₃, water, 10% Na₂S₂O₃, water and saturated NaCl, dried over anhydrous Na₂SO₄, filtered and concentrated. The residue was purified by chromatography (40% acetone/hexane) to give the title compound (47 mg, 0.080 mmol, 18%). ¹H NMR (CDCl₃, MeOH-d₄): 8.08 (s, 2H), 7.60 (dd, 1H, J=7.5, 1.5 Hz), 7.54 (m, 1H), 7.12 (m, 1H), 7.04 (d, 1H, J=8.4 Hz), 3.89 (s, 3H), 3.78 (s, 3H).

### EXAMPLE 80

### 6-(4-Isopropylaminophenyl)-3-(2-methoxyphenyl)-7H-[1,2,4]triazolo[3,4-b][1,3,4]thiadiazine

To a solution of 6-(4-aminophenyl)-3-(2-methoxyphenyl)-7H-[1,2,4]triazolo[3,4-b][1,3,4]thiadiazine (30 mg, 0.089 mmol) in methanol (4 mL) was added acetone (0.5 mL) and one drop of glacial acetic acid. The reaction mixture was cooled to 0° C., then was added sodium cyanoborohydride (25 mg×3). The reaction mixture was stirred at 0° C. for 1 h and warmed to room temperature. The mixture was diluted with 50 mL of ethyl acetate and washed with saturated NaHCO₃ (50 mL). The organic layer was dried over anhydrous Na₂SO₄, filtered and concentrated. The residue was purified by chromatography (40% acetone/hexane) to give the title compound (24 mg, 0.063 mmol, 71%). ¹H NMR (CDCl₃): 7.60-7.66 (m, 3H), 7.48 (m, 1H), 7.08 (dt, 1H, J=7.2, 0.6 Hz), 7.01 (d, 1H, J=8.1 Hz), 6.54 (m, 2H), 3.90 (s, 2H), 3.75 (s, 3H), 3.68 (m, 1H), 1.25 (d, 6H, J=6.0 Hz).

### EXAMPLE 81

### 3-(4-Chloro-2-methylphenyl)-6-(4-methylphenyl)-7H-[1,2,4]triazolo[3,4-b][1,3,4]thiadiazine

(a) 4-Chloro-2-methylbenzoyl-2-dithiocarboxyhydrazide, potassium salt. To a mixture of 4-chloro-2-methylbenzohydrazide (1.84 g, 10 mmol) in 40 mL of absolute ethanol at room temperature was added KOH (0.84 g, 15 mmol), followed by carbon disulfide (0.9 mL, 15 mmol) and the mixture was stirred at room temperature for 4 h. The precipitated product was collected, washed with cold ethanol and dried under vacuum (2.10 g, 7.0 mmol, 70%) to give the title compound as solids. ¹H NMR (DMSO-d₆): 10.0 (s, 1H, br), 9.57 (s, 1H, br), 7.61 (d, 1H, J=7.8 Hz), 7.29-7.34 (m, 3H), 2.39 (s, 3H).

(b) 4-Amino-5-(4-chloro-2-methylphenyl)-3-mercapto-(4H)-1,2,4-triazole. A mixture of 4-chloro-2-methyl benzoyl-2-dithiocarboxyhydrazide, potassium salt and hydrazine hydrate (3 mL, 62 mmol) in ethanol (30 mL) and water (10 mL) was heated at 80° C. for 3 h. The reaction mixture was cooled to room temperature, diluted with 100 mL of water and acidified with concentrated HCl. The white precipitate was collected, washed with water and dried under vacuum. A small portion of the product was recrystallized using ethanol and hexane to give title compound (69 mg, 0.29 mmol). ¹H NMR (DMSO-d₆): 7.53 (d, 1H, J=8.1 Hz), 7.49 (d, 1H, J=2.1 Hz), 7.41 (dd, 1H, J=8.4, 2.1 Hz), 5.57 (s, 2H, br), 2.28 (s, 3H).

(c) 3-(4-Chloro-2-methylphenyl)-6-(4-methylphenyl)-7H-[1,2,4]triazolo[3,4-b][1,3,4]thiadiazine: The title compound was prepared in a manner similar to example 23. From 2-bromo-1-(4-methylphenyl)ethanone (63 mg, 0.30 mmol) and 4-amino-5-(4-chloro-2-methylphenyl)-3-mercapto-(4H)-1,2,4-triazole (61 mg, 0.25 mmol) was obtained the title compound as a white solid (32 mg, 0.09 mmol, 36%). ¹H NMR (DMSO-d₆): 7.78 (m, 2H), 7.55 (m, 2H), 7.45 (dd, 1H, J=8.1, 1.5 Hz), 7.34 (m, 2H), 4.44 (s, 2H), 2.38 (s, 3H), 2.37 (s, 3H).

### EXAMPLE 82

### 3-(2-Methoxyphenyl)-6-(3-methylphenyl)-7H-[1,2,4]triazolo[3,4-b][1,3,4]thiadiazine

The title compound was prepared in a manner similar to example 23. From 2-bromo-1-(3-methylphenyl)ethanone (145 mg, 0.68 mmol) and 4-amino-5-(2-methoxylphenyl)-3-mercapto-(4H)-1,2,4-triazole (140 mg, 0.63 mmol) was obtained the title compound as a fluffy white solid (32 mg, 0.47 mmol, 75%). ¹H NMR (CDCl₃): 7.62 (dd, 1H, J=7.5, 1.5 Hz), 7.59 (m, 2H), 7.50 (ddd, 1H, J=8.7, 7.5, 1.8 Hz), 7.33 (m, 2H), 7.09 (dt, 1H, J=7.2, 0.9 Hz), 7.04 (d, 1H, J=8.1 Hz), 3.98 (s, 2H), 3.75 (s, 3H), 2.39 (s, 3H).

### EXAMPLE 83

### 3-(2-Methylfuran-3-yl)-6-(4-methylphenyl)-7H-[1,2,4]triazolo[3,4-b][1,3,4]thiadiazine

The title compound was prepared in a manner similar to example 23. From 2-bromo-1-(4-methylphenyl)ethanone (360 mg, 1.70 mmol) and 4-amino-5-(2-methyl-3-furyl)-4H-1,2,4-triazole-3-thiol (300 mg, 1.53 mmol) was obtained the title compound as a white solid (549 mg, 1.41 mmol, 92%). ¹H NMR (DMSO-d₆): 7.91 (m, 2H), 7.72 (d, 1H, J=1.8 Hz), 7.40 (m, 2H), 6.97 (d, 1H, J=2.1 Hz), 4.41 (s, 2H), 2.58 (s, 3H), 2.40 (s, 3H).

### EXAMPLE 84

### 3-(2-Methylaminophenyl)-6-(4-methylphenyl)-7H-[1,2,4]triazolo[3,4-b][1,3,4]thiadiazine

To a mixture of 3-(2-aminophenyl)-6-(4-methylphenyl)-7H-[1,2,4]triazolo[3,4-b][1,3,4]thiadiazine (24 mg, 0.07 mmol) in methanol (5 mL) was added 6 drops of acetic acid and 37% aqueous formaldehyde (22.4 mg, 0.74 mmol) and it was stirred at room temperature for 1 h and was then cooled to 0° C. To the mixture was added sodium borohydride cyanate (46.6 mg, 0.74 mmol) and it was stirred at room temperature overnight. It was evaporated and the residue was poured into ethyl acetate (10 mL) and washed with water (30 mL), evaporated and the residue was purified by column chromatography to give 1 mg (4%) of the title compound. ¹H NMR (CDCl₃): 7.92-7.89 (dd, J1=7.8 Hz, J2=1.2 Hz, 1H), 7.82 (d, J=8.4 Hz, 2H), 7.71 (bs, 1H), 7.32 (d, J=8.4 Hz, 2H), 6.78 (d, J1=5.4 Hz, 1H), 6.72-6.67 (t, J=6.9 Hz, 1H), 3.97 (s, 2H), 2.98 (d, J=4.5 Hz, 3H), 2.44 (s, 3H).

### EXAMPLE 85

### 6-(4-Methoxycarbonylphenyl)-3-(2-methoxyphenyl)-7H-[1,2,4]triazolo[3,4-b][1,3,4]thiadiazine

The title compound was prepared in a manner similar to example 2. From 2-bromo-1-(4-methoxycarbonylphenyl)ethanone (1.0 g, 3.89 mmol) and 4-amino-5-(2-methoxyphenyl)-3-mercapto-(4H)-1,2,4-triazole (0.86 g, 3.89 mmol) was obtained 1.13 g (76%) of the title compound. ¹H NMR (CDCl₃): 8.11 (d, J=8.7 Hz, 2H), 7.86 (d, J=9.0 Hz, 2H), 7.64-7.61 (dd, J1=7.5 Hz, J2=1.5 Hz, 1H), 7.55-7.49 (m, 1H), 7.64-7.61 (m, 1H), 7.02 (d, J=8.4 Hz, 1H), 4.02 (s, 2H), 3.95 (s, 3H), 3.74 (s, 3H).

### EXAMPLE 86

### 3-(2-Methoxyphenyl)-6-(1-methyl-1H-pyrrol-3-yl)-7H-[1,2,4]triazolo[3,4-b][1,3,4]thiadiazine

The title compound was prepared in a manner similar to example 2. From 2-chloro-1-(1-methyl-1H-pyrrol-3-yl)-ethanone (158 mg, 1 mmol) and 4-amino-5-(2-methoxyphenyl)-3-mercapto-(4H)-1,2,4-triazole (230 mg, 1 mmol) was obtained 0.25 g (87%) of the title compound. ¹H NMR (CD₃OD): 8.05 (d, J=7.5 Hz, 1H), 7.75 (t, J=7.8 Hz, 1H), 7.60 (s, 1H), 7.35 (d, J=8.4 Hz, 2H), 7.25 (t, J=7.8 Hz, 1H), 6.85 (t, J=2.7 Hz, 1H), 6.26 (m, 1H), 4.27 (s, 2H), 4.03 (s, 3H), 3.99 (s, 3H), 3.75 (s, 3H).

### EXAMPLE 87

### 6-(4-Cyanophenyl)-3-(2-methoxyphenyl)-7H-[1,2,4]triazolo[3,4-b][1,3,4]thiadiazine

The title compound was prepared in a manner similar to example 2. From 2-bromo-1-(4-cyanophenyl)ethanone (1.0 g, 4.46 mmol) and 4-amino-5-(2-methoxyphenyl)-3-mercapto-(4H)-1,2,4-triazole (0.99 g, 4.46 mmol) was obtained 1.34 g (87%) of the title compound. ¹H NMR (CDCl₃): 7.90 (d, J=8.4 Hz, 2H), 7.75 (d, J=8.1 Hz, 2H), 7.63-7.59 (dd, J1=7.5 Hz, J2=1.8 Hz, 1H), 7.56-7.45 (m, 1H), 7.14-7.09 (t, J=7.8 Hz, 1H), 7.03 (d, J=8.4 Hz, 1H), 4.01 (s, 2H), 3.74 (s, 3H).

### EXAMPLE 88

### 5,6-Dihydro-3-(2-methoxyphenyl)-6-(4-methylphenyl)-7H-[1,2,4]triazolo[3,4-b][1,3,4]thiadiazine

To a solution of 3-(2-methoxyphenyl)-6-(4-methylphenyl)-7H-[1,2,4]triazolo[3,4-b][1,3,4]thiadiazine in a dioxane (8 mL) and water (3 mL) was added sodium borohydride (304 mg, 8 mmol). The solution was stirred at room temperature overnight. It was then cooled to 0° C. and quenched with 2N hydrochloride, and it was adjusted to pH=10 by addition of 2N sodium carbonate. The solution was extracted with ethyl acetate (3×20 mL). The extracts were dried and concentrated and the solid was washed with ethyl acetate (2×1 mL) to give 210 mg (62%) of title compound. ¹H NMR (CD₃OD): 7.58-7.51 (m, 1H), 7.43-7.39 (dd, J1=7.2 Hz, J2=1.5 Hz, 1H), 7.22-7.06 (m, 6H), 4.54-4.50 (dd, J1=8.4 Hz, J2=2.7 Hz, 1H), 3.87 (s, 2H), 3.72-3.48 (m, 2H), 2.29 (s, 3H).

### EXAMPLE 89

### 6-(4-Carboxyphenyl)-3-(2-Methoxyphenyl)-7H-[1,2,4]triazolo[3,4-b][1,3,4]thiadiazine

To a solution of 6-(4-methoxycarbonylphenyl)-3-(2-methoxyphenyl)-7H-[1,2,4]triazolo[3,4-b][1,3,4]thiadiazine (60 mg) in methanol (5 mL) was added sodium hydroxide (150 mg) in water (5 mL). It was stirred at room temperature for 2 h, then was neutralized with 2N hydrochloride. The solution was extracted with ethyl acetate (3×20 mL). The extracts were dried, concentrated to give 55 mg (86%) of the title compound. ¹H NMR (CD₃OD): 8.10 (d, J=8.7 Hz, 2H), 7.98 (d, J=8.4 Hz, 2H), 7.62-7.54 (m, 2H), 7.19 (d, J=8.4 Hz, 1H), 7.16-7.10 (m, 1H), 4.37 (s, 2H), 3.78 (s, 3H).

### EXAMPLE 90

### 3-(4-Chloro-2-methylphenyl)-6-(4-methyl-3-nitrophenyl)-7H-[1,2,4]triazolo[3,4-b][1,3,4]thiadiazine

The title compound was prepared in a manner similar to example 45b. From 2-bromo-1-(4-methyl-3-nitrophenyl)ethanone (0.107 g, 0.416 mmol) and 4-amino-5-(4-chloro-2-methylphenyl)-4H-[1,2,4]triazolo-3-thiol (0.100 g, 0.416 mmol) was obtained 0.177 g (88%) of the title compound as a white solid: ¹H NMR (DMSO-d₆) 8.45 (d, J=1.9 Hz, 1H), 8.09 (dd, J=8.1 and 2.1 Hz, 1H), 7.68 (d, J=8.5 Hz, 1H), 7.55 (d, J=8.2 Hz, 1H), 7.55 (d, J=1.9 Hz, 1H), 7.47-7.43 (m, 1H), 4.51 (s, 2H), 2.57 (s, 3H), 2.40 (s, 3H).

### EXAMPLE 91

### 6-(3-Amino-4-methylphenyl)-3-(4-chloro-2-methylphenyl)-7H-[1,2,4]triazolo[3,4-b][1,3,4]thiadiazine

The title compound was prepared in a manner similar to example 58. From 3-(4-chloro-2-methylphenyl)-6-(4-methyl-3-nitrophenyl)-7H-[1,2,4]triazolo[3,4-b][1,3,4]thiadiazine (0.100 g, 0.208 mmol) and tin (II) chloride dihydrate (0.211 g, 0.936 mmol) was obtained 0.023 g (30%) of the title compound as a white solid: ¹H NMR (DMSO-d₆) 7.53 (d, J=8.7 Hz, 1H), 7.53 (d, J=2.4 Hz, 1H), 7.45 (dd, J=8.7 and 2.4 Hz, 1H), 7.08-7.06 (m, 2H), 7.00 (dd, J=7.8 and 1.8 Hz, 1H), 5.15 (br s, 2H), 4.34 (s, 2H), 2.38 (s, 3H), 2.10 (s, 3H).

### EXAMPLE 92

### 6-(4-Methylphenyl)-3-(1H-pyrrol-2-yl)-7H-[1,2,4]triazolo[3,4-b][1,3,4]thiadiazine

(a) 1H-Pyrrole-2-carbonyl-2-dithiocarboxyhydrazide, potassium salt. To an oven-dried round bottom reaction flask charged with a magnetic stir bar under argon at room temperature with 1H-pyrrole-2-carbohydrazide (0.500 g, 4.00 mmol) and anhydrous ethanol (8.0 mL) was added potassium hydroxide (0.337 g, 6.00 mmol) to form a yellow solution. The solution was stirred for 15 min at rt followed by addition of carbon disulfide (0.36 mL, 6.0 mmol) and additional ethanol (5.0 mL). The reaction mixture to stirred for 12 h, the resulting precipitate was filtered and collected on a Buchner funnel and then dried overnight under vacuum to give 0.830 g (87%) of the title compound as a yellow solid: ¹H NMR (DMSO-d₆) 11.55 (br s, 1H), 9.86 (br s, 1H), 9.57 (br s, 1H), 6.88-6.87 (m, 1H), 6.79 (d, J=1.1 Hz, 1H), 6.08 (dd, J=2.3 and 1.2 Hz, 1H).

(b) 4-Amino-5-(1H-pyrrol-2-yl)-4H-[1,2,4]triazolo-3-thiol. To an oven-dried round bottom reaction flask charged with a magnetic stir bar under argon at rt with 1H-pyrrole-2-carbonyl-2-dithiocarboxyhydrazide, potassium salt (0.830 g, 3.48 mmol) in ethanol (17.4 mL) and water (5.0 mL) was added anhydrous hydrazine (1.51 mL, 41.8 mmol). The yellow solution was heated at 100° C. for 5 h and then cooled to rt. The yellow solution was diluted with water (10 ml), acidified to pH=1 using concentrated HCl and the resulting precipitate was filtered and collected on a Buchner funnel to give 0.299 g (48%) of the crude product as a white solid. It was recrystallized (ethanol:water; 10 mL:1 mL) to yield 0.110 g (18%) of the title compound as a white sold: ¹H NMR (DMSO-d₆) 13.67 (br s, 1H), 11.62 (br s, 1H), 7.01-6.95 (m, 2H), 6.20-6.18 (m, 1H), 5.78 (br s, 2H).

(c) 6-(4-Methylphenyl)-3-(1H-pyrrol-2-yl)-7H-[1,2,4]triazolo[3,4-b][1,3,4]thiadiazine. The title compound was prepared in a manner similar to example 45b. From 2-bromo-1-(4-methylphenyl)ethanone (0.11 g, 0.50 mmol) and 4-amino-5-(1H-pyrrol-2-yl)-4H-[1,2,4]triazolo-3-thiol (0.091 g, 0.50 mmol) was obtained 0.172 g (91%) of the title compound as a yellow solid: ¹H NMR (DMSO-d₆) 11.82 (br s, 1H), 7.97 (d, J=8.1 Hz, 2H), 7.42 (d, J=8.4 Hz, 2H), 7.02-7.01 (m, 1H), 6.92-6.90 (m, 1H), 6.27-6.26 (m, 1H), 4.42 (s, 2H), 2.41 (s, 3H).

### EXAMPLE 93

### 3-(4-Methyl-1,2,3-thiadiazol-5-yl)-6-(4-methylphenyl)-7H-[1,2,4]triazolo[3,4-b][1,3,4]thiadiazine

(a) 4-Methyl-1,2,3-thiadiazole-5-carbonyl-2-dithiocarboxyhydrazide, potassium salt. The title compound was prepared in a manner similar to example 92a. From 4-methyl-1,2,3-thiadiazole-5-carbohydrazide (0.500 g, 3.16 mmol) and carbon disulfide (0.29 mL, 4.7 mmol) was obtained 0.666 g (82%) of the title compound as a yellow solid: ¹H NMR (DMSO-d₆) 9.94 (br s, 1H), 9.76 (br s, 1H), 2.84 (s, 3H).

(b) 4-Amino-5-(4-methyl-1,2,3-thiadiazol-5-yl)-4H-[1,2,4]triazolo-3-thiol. The title compound was prepared in a manner similar to example 92b. From 4-methyl-1,2,3-thiadiazole-5-carbonyl-2-dithiocarboxyhydrazide, potassium salt (0.663 g, 2.57 mmol) and anhydrous hydrazine (1.12 mL, 30.8 mmol) was obtained 0.097 g (18%) of the title compound as a white sold: ¹H NMR (DMSO-d₆) 14.35 (br s, 1H), 6.09 (br s, 2H), 2.95 (s, 3H).

(c) 3-(4-Methyl-1,2,3-thiadiazol-5-yl)-6-(4-methylphenyl)-7H-[1,2,4]triazolo[3,4-b][1,3,4]thiadiazine. The title compound was prepared in a manner similar to example 45b. From 2-bromo-1-(4-methylphenyl)ethanone (0.095 g, 0.44 mmol) and 4-amino-5-(4-methyl-1,2,3-thiadiazol-5-yl)-4H-[1,2,4]triazolo-3-thiol (0.095 g, 0.44 mmol) was obtained 0.136 g (75%) of the title compound as a white solid; ¹H NMR (DMSO-d₆) 8.05 (d, J=8.1 Hz, 2H), 7.48 (d, J=8.4 Hz, 2H), 4.52 (s, 2H), 3.10 (s, 3H), 2.44 (s, 3H).

### EXAMPLE 94

### 3-(1-Methyl-1H-pyrrol-2-yl)-6-(4-methylphenyl)-7H-[1,2,4]triazolo[3,4-b][1,3,4]thiadiazine

(a) 1-Methyl-1H-pyrrole-2-carbonyl-2-dithiocarboxyhydrazide, potassium salt. The title compound was prepared in a manner similar to example 92a. From 1-methyl-1H-pyrrole-2-carbohydrazide (0.500 g, 3.59 mmol) and carbon disulfide (0.32 mL, 5.4 mmol) was obtained 0.866 g (95%) of the title compound as a yellow solid: ¹H NMR (DMSO-d₆) 9.88 (br s, 1H), 9.61 (br s, 1H), 6.94-6.92 (m, 1H), 6.79-6.77 (m, 1H), 6.03-6.01 (m, 1H), 3.82 (s, 3H).

(b) 4-Amino-5-(1-methyl-1H-pyrrol-2-yl)-4H-[1,2,4]triazolo-3-thiol. The title compound was prepared in a manner similar to example 92b. From 1-methyl-1H-pyrrole-2-carbonyl-2-dithiocarboxyhydrazide, potassium salt (0.850 g, 3.35 mmol) and anhydrous hydrazine (1.46 mL, 40.2 mmol) was obtained 0.172 g (26%) of the title compound as a white sold: ¹H NMR (DMSO-d₆) 13.79 (br s, 1H), 7.02 (d, J=3.3 Hz, 2H), 6.15 (t, J=3.3 Hz, 1H), 5.74 (br s, 2H), 3.79 (s, 3H).

(c) 3-(1-Methyl-1H-pyrrol-2-yl)-6-(4-methylphenyl)-7H-[1,2,4]triazolo[3,4-b][1,3,4]thiadiazine. The title compound was prepared in a manner similar to example 45b. From 2-bromo-1-(4-methylphenyl)ethanone (0.098 g, 0.46 mmol) and 4-amino-5-(1-methyl-1H-pyrrol-2-yl)-4H-[1,2,4]triazolo-3-thiol (0.090 g, 0.46 mmol) was obtained 0.117 g (65%) of the title compound as a yellow solid: ¹H NMR (DMSO-d₆) 7.92 (d, J=8.4 Hz, 2H), 7.40 (d, J=8.1 Hz, 2H), 7.08 (t, J=2.2 Hz, 1H), 6.84 (dd, J=4.0 and 1.8 Hz, 1H), 6.22 (dd, J=4.0 and 2.6 Hz, 1H), 4.40 (s, 2H), 3.95 (s, 3H), 2.40 (s, 3H).

### EXAMPLE 95

### 3-(1-Methyl-4-nitro-1H-pyrrol-2-yl)-6-(4-methylphenyl)-7H-[1,2,4]triazolo[3,4-b][1,3,4]thiadiazine

(a) 1-Methyl-4-nitro-1H-pyrrole-2-carbonyl-2-dithiocarboxyhydrazide, potassium salt. The title compound was prepared in a manner similar to example 92a. From 1-methyl-4-nitro-1H-pyrrole-2-carbohydrazide (0.500 g, 2.71 mmol) and carbon disulfide (0.25 mL, 4.1 mmol) was obtained 0.685 g (85%) of the title compound as a brown solid: ¹H NMR (DMSO-d₆) 10.16 (br s, 1H), 9.63 (br s, 1H), 8.14 (s, 1H), 7.46 (s, 1H), 3.90 (s, 3H).

(b) 4-Amino-5-(1-methyl-4-nitro-1H-pyrrol-2-yl)-4H-[1,2,4]triazolo-3-thiol. The title compound was prepared in a manner similar to example 92b. From 1-methyl-4-nitro-1H-pyrrole-2-carbonyl-2-dithiocarboxyhydrazide, potassium salt (0.683 g, 2.29 mmol) and anhydrous hydrazine (1.00 mL, 27.5 mmol) was obtained 0.225 g (41%) of the title compound as a white sold: ¹H NMR (DMSO-d₆) 14.07 (br s, 1H), 8.28 (d, J=2.2 Hz, 1H), 7.61 (d, J=2.2 Hz, 1H), 5.81 (br s, 2H), 3.88 (s, 3H).

(c) 3-(1-Methyl-4-nitro-1H-pyrrol-2-yl)-6-(4-methylphenyl)-7H-[1,2,4]triazolo[3,4-b][1,3,4]thiadiazine. The title compound was prepared in a manner similar to example 45b. From 2-bromo-1-(4-methylphenyl)ethanone (0.111 g, 0.520 mmol) and 4-amino-5-(1-methyl-4-nitro-1H-pyrrol-2-yl)-4H-[1,2,4]triazolo-3-thiol (0.125 g, 0.520 mmol) was obtained 0.185 g (82%) of the title compound as a yellow solid: ¹H NMR (DMSO-d₆) 8.33 (d, J=1.5 Hz, 1H), 7.92 (d, J=8.0 Hz, 2H), 7.42 (d, J=8.0 Hz, 2H), 7.32 (t, J=1.8 Hz, 1H), 4.42 (s, 2H), 4.03 (s, 3H), 2.41 (s, 3H).

### EXAMPLE 96

### 6-(7-Bromoindolin-5-yl)-3-(2-methoxyphenyl)-7H-[1,2,4]triazolo[3,4-b][1,3,4]thiadiazine

(a) 2-Bromo-1-(7-bromoindolin-5-yl)ethanone. The title compound was prepared in a manner similar to example 44a. From 1-(2,3-dihydro-1H-indol-5-yl)ethanone (0.500 g, 3.10 mmol) and bromine (0.16 mL, 3.1 mmol) was obtained 0.040 g (5%) of the title compound as a yellow solid: ¹H NMR (CDCl₃): 7.87 (d, J=1.8 Hz, 1H), 7.65-7.64 (m, 1H), 4.32 (s, 2H), 3.81-3.75 (m, 2H), 3.26-3.20 (m, 2H).

(b) 6-(7-Bromoindolin-5-yl)-3-(2-methoxyphenyl)-7H-[1,2,4]triazolo[3,4-b][1,3,4]thiadiazine. The title compound was prepared in a manner similar to example 40. From 2-bromo-1-(7-bromoindolin-5-yl)ethanone (0.055 g, 0.17 mmol) and 4-amino-5-(2-methoxyphenyl)-4H-[1,2,4]triazolo-3-thiol (0.038 g, 0.17 mmol) was obtained 0.005 g (7%) of the title compound as a yellow solid: ¹H NMR (DMSO-d₆) 7.70-7.69 (m, 1H), 7.60-7.54 (m, 1H), 7.49-7.46 (m, 2H), 7.22 (d, J=8.1 Hz, 1H), 7.11 (t, J=7.5 Hz, 1H), 6.50 (br s, 1H), 4.29 (s, 2H), 3.73 (s, 3H), 3.61-3.55 (m, 2H), 3.12-3.06 (m, 2H).

### EXAMPLE 97

### 3-(4-Chloro-2-methoxyphenyl)-6-(4-methylphenyl)-7H-[1,2,4]triazolo[3,4-b][1,3,4]thiadiazine

(a) Methyl 4-chloro-2-methoxybenzoate. A mixture of 4-chloro-2-methoxybenzoic acid (5.02 g, 6.90 mmol) in 75 mL of methanol and 1 mL of concentrated sulfuric acid was refluxed overnight. The reaction mixture was cooled to room temperature and methanol was removed under vacuum. The residue was dissolved in 200 mL of ethyl acetate and washed with saturated NaHCO₃ (150 mL×2). The organic layer was dried under anhydrous Na₂SO₄, filtered and concentrated to obtain title product as a clear oil (5.19 g, 6.62 mmol, 96%). ¹H NMR (CDCl₃): 7.62 (d, 1H, J=8.6 Hz), 7.01 (d, 1H, J=2.4 Hz), 6.98 (dd, 1H, J=1.9, 8.8 Hz), 3.88 (s, 3H).

(b) 4-Chloro-2-methoxybenzohydrazide. A mixture of methyl 4-chloro-2-methoxybenzoate (6.0 g, 29.9 mmol), methanol (50 mL) and hydrazine hydrate (6.0 mL, 123.3 mmol) was refluxed overnight. The reaction mixture was cooled to room temperature and the precipitate was collected by filtration and further dried under vacuum to give the title compound as white solids (4.36 g, 21.7 mmol, 73%). ¹H NMR (DMSO-d₆): 9.24 (broad, s, 1H), 7.65 (d, 1H, J=8.4 Hz), 7.19 (d, 1H, J=2.1 Hz), 7.08 (dd, 1H, J=1.8, 8.1 Hz), 4.53 (d, 2H, J=4.2 Hz), 3.88 (s, 3H).

(c) 4-Chloro-2-methoxyl benzoyl-2-dithiocarboxyhydrazide, potassium salt. The title compound was prepared in a manner similar to example 81a. From 4-chloro-2-methoxybenzohydrazide (1.5 g, 7.48 mmol) and carbon disulfide (0.8 mL, 13.3 mmol) was obtained the title compound as an off-white solid (549 mg, 6.74 mmol, 90%). ¹H NMR (DMSO-d₆): 11.91 (s, 1H), 9.80 (s, 1H), 7.99 (d, 1H, J=8.4 Hz), 7.29 (d, 1H, J=1.8 Hz), 7.16 (dd, 1H, J=2.4, 8.7 Hz), 3.99 (s, 3H).

(d) 4-Amino-5-(4-chloro-2-methoxylphenyl)-4H-1,2,4-triazole-3-thiol. The title compound was prepared in a manner similar to example 81b. From 4-chloro-2-methoxylbenzoyl-2-dithiocarboxyhydrazide, potassium salt (1.05 g, 3.33 mmol) and hydrazine hydrate (3.00 mL, 61.5 mmol) was obtained the title compound as a white solid (666 mg). The crude product was carried on to next step without further purification.

(e) 3-(4-Chloro-2-methoxyphenyl)-6-(4-methylphenyl)-7H-[1,2,4]triazolo[3,4-b][1,3,4]thiadiazine. The title compound was prepared in a manner similar to example 23 using the crude product from above (201 mg) and 2-bromo-1-(4-methylphenyl)ethanone (254 mg, 1.20 mmol). The crude product was purified by chromatography (70% ethyl acetate/hexane) to give the title compound as a yellow solid (63 mg, 0.17 mmol). ¹H NMR (CDCl₃): 7.68 (m, 2H), 7.55 (d, 1H, J=7.8 Hz), 7.27 (m, 2H), 7.09 (dd, 1H, J=2.1, 8.1 Hz), 7.01 (d, 1H, J=1.5 Hz), 3.98 (s, 2H), 3.75 (s, 3H), 2.42 (s, 3H).

### EXAMPLE 98

### 3-(2-Methyl-imidazo[1,2-a]pyridin-3-yl)-6-(4-methylphenyl)-7H-[1,2,4]triazolo[3,4-b][1,3,4]thiadiazine

(a) 4-Amino-5-(2-methyl-imidazo[1,2-a]pyridin-3-yl)-4H-[1,2,4]triazolo-3-thiol. The title compound was prepared in a manner similar to example 92a and 92b. From 2-methyl-imidazo[1,2-a]pyridine-3-carboxylic acid hydrazide (0.515 g, 2.71 mmol) and carbon disulfide (0.250 mL, 4.07 mmol) was obtained 0.553 g (67%) of the 2-methyl-imidazo[1,2-a]pyridine-3-carbonyl-3-dithiocarboxyhydrazide, potassium salt as a yellow solid. It was then reacted with hydrazine (0.790 mL, 21.7 mmol) to yield 0.102 g (23%) of the title compound as a white sold: ¹H NMR (DMSO-d₆) 14.41 (br s, 1H), 8.79 (dd, J=6.8 and 0.9 Hz, 1H), 7.98-7.95 (m, 2H), 7.45 (td, J=6.5 and 2.1 Hz, 1H), 5.64 (br s, 2H), 2.56 (s, 3H).

(b) 3-(2-Methyl-imidazo[1,2-a]pyridin-3-yl)-6-(4-methylphenyl)-7H-[1,2,4]triazolo[3,4-b][1,3,4]thiadiazine. The title compound was prepared in a manner similar to example 45b. From 2-bromo-1-(4-methylphenyl)ethanone (0.078 g, 0.36 mmol) and 4-amino-5-(2-methyl-imidazo[1,2-a]pyridin-3-yl)-4H-[1,2,4]triazolo-3-thiol (0.090 g, 0.36 mmol) was obtained 0.022 g (17%) of the title compound as a white solid: ¹H NMR (DMSO-d₆) 8.94 (dd, J=7.0 and 1.1 Hz, 1H), 8.00-7.90 (m, 2H), 7.80 (d, J=8.1, 2H), 7.45 (t, J=6.8 Hz, 1H), 7.33 (d, J=8.4 Hz, 2H), 4.56 (s, 2H), 2.55 (s, 3H), 2.36 (s, 3H).

### EXAMPLE 99

### 3-(1-Methyl-4-amino-1H-pyrrol-2-yl)-6-(4-methylphenyl)-7H-[1,2,4]triazolo[3,4-b][1,3,4]thiadiazine

The title compound was prepared in a manner similar to example 58. From 3-(1-methyl-4-nitro-1H-pyrrol-2-yl)-6-(4-methylphenyl)-7H-[1,2,4]triazolo[3,4-b][1,3,4]thiadiazine (0.047 g, 0.13 mmol) and tin (II) chloride dihydrate (0.135 g, 0.597 mmol) was obtained 0.020 g (46%) of the title compound as a brown solid: ¹H NMR (DMSO-d₆) 8.33 (d, J=1.8 Hz, 1H), 7.92 (d, J=8.1 Hz, 2H), 7.42 (d, J=8.4 Hz, 2H), 7.32 (d, J=1.8 Hz, 1H), 4.43 (s, 2H), 4.03 (s, 3H), 2.41 (s, 3H).

### EXAMPLE 100

### Identification of 3-(2-Chlorophenyl)-6-(3,4-methylenedioxyphenyl)-7H-[1,2,4]triazolo[3,4-b][1,3,4]thiadiazine and Analogs as Caspase Cascade Activators and Inducers of Apoptosis in Solid Tumor Cells

Human breast cancer cell lines T-47D, human hepatocellular carcinoma cell line SNU398, human colon carcinoma cell line HCT116, human cancer cell line H1299, human Burkitt's lymphoma cell line Namalwa, human lymphoma cell line Raji, and human B cell lymphoblastoid cell line Ramos were grown according to media component mixtures designated by American Type Culture Collection+10% FCS (Invitrogen Corporation), in a 5% CO₂-95% humidity incubator at 37° C. T-47D and H1299 cells were maintained at a cell density between 50 and 80% confluency at a cell density of 0.1 to 0.6×10⁶ cells/mL. Cells were harvested at 600×g and resuspended at 0.65×10⁶ cells/mL into appropriate media+10% FCS. An aliquot of 22.5 μL of cells was added to a well of a 384-well microtiter plate containing 2.5 μL of a 10% DMSO in RPMI-1640 media solution containing 0.16 to 100 μM of 3-(2-chlorophenyl)-6-(3,4-methylenedioxyphenyl)-7H-[1,2,4]triazolo[3,4-b][1,3,4]thiadiazine or other test compound (0.016 to 10 μM final). An aliquot of 22.5 μL of cells was added to a well of a 384-well microtiter plate containing 2.5 μL of a 10% DMSO in RPMI-1640 media solution without test compound as the control sample. The samples were mixed by agitation and then incubated at 37° C. for 48 h in a 5% CO₂-95% humidity incubator. After incubation, the samples were removed from the incubator and 25 μL of a solution containing 14 μM of N—(Ac-DEVD)-N′-ethoxycarbonyl-R110 (SEQ ID No.:1) fluorogenic substrate (Cytovia, Inc.; WO99/18856), 20% sucrose (Sigma), 20 mM DTT (Sigma), 200 mM NaCl (Sigma), 40 mM Na PIPES buffer pH 7.2 (Sigma), and 500 μg/mL lysolecithin (Calbiochem) was added. The samples were mixed by agitation and incubated at room temperature. Using a fluorescent plate reader (Model SPECTRAfluor Plus, Tecan), an initial reading (T=0) was made approximately 1-2 min after addition of the substrate solution, employing excitation at 485 nm and emission at 530 nm, to determine the background fluorescence of the control sample. After the 3 h incubation, the samples were read for fluorescence as above (T=3 h).

Calculation:

The Relative Fluorescence Unit values (RFU) were used to calculate the sample readings as follows:
RFU_{(T=3H)}−Control RFU_{(T=0)}=Net RFU_{(T=3h)}

The activity of caspase cascade activation was determined by the ratio of the net RFU value for 3-(2-chlorophenyl)-6-(3,4-methylenedioxyphenyl)-7H-[1,2,4]triazolo[3,4-b][1,3,4]thiadiazine (Example P) or other test compound to that of control samples. The EC₅₀ (nM) was determined by a sigmoidal dose-response calculation (Prism 3.0, GraphPad Software Inc.).

The caspase potency (EC₅₀) are summarized in Table I:

| Ex- | | | | | | | | |
|---|---|---|---|---|---|---|---|---|
| am- | | | | | | | | |
| ple | Structure | T47D | SNU398 | HCT116 | H1299 | Namalwa | RAJI | RAMOS |
| A | 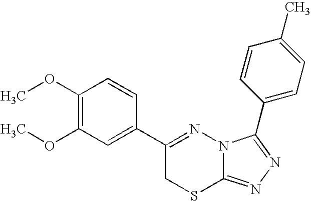 | 2425 | ND | ND | 2728 | ND | ND | ND |
| B | 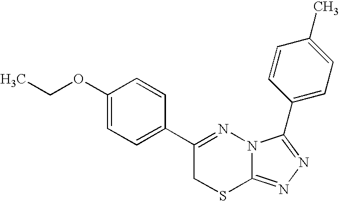 | 2711 | 266 | 431 | 1251 | 270 | 455 | 331 |
| C | 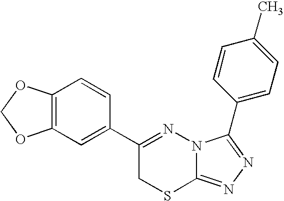 | 683 | 599 | >10000 | ND | 580 | 568 | 711 |
| D | 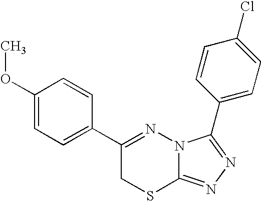 | 315 | 82 | 130 | 142 | 58 | 84 | 63 |
| E | 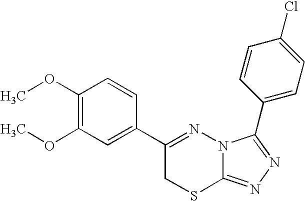 | 1876 | ND | ND | 2096 | ND | ND | ND |
| F | 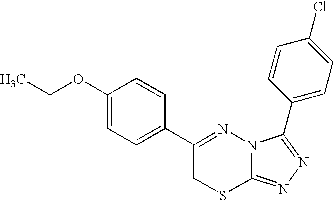 | 561 | 89 | 141 | 275 | 69 | 155 | 106 |
| G | 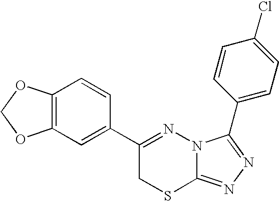 | 513 | 273 | 849 | 1420 | 265 | 393 | 481 |
| H | 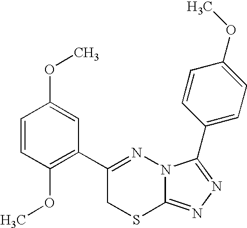 | 2440 | 1229 | >10000 | 2425 | 1176 | 2327 | 1266 |
| I | 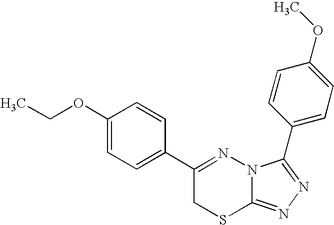 | 2487 | 1008 | 1577 | 1817 | ND | ND | ND |
| J | 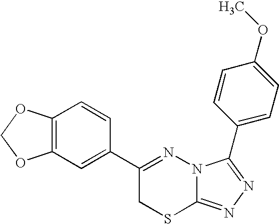 | 3274 | 2551 | >10000 | 2933 | >10000 | >1000 | >1000 |
| K | 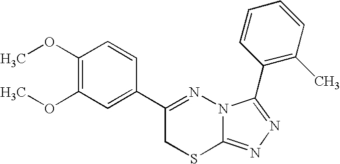 | 5131 | 316 | 578 | 697 | 267 | 479 | 272 |
| L | 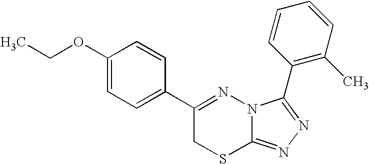 | 1338 | 92 | 126 | 339 | 62 | 141 | 93 |
| M | 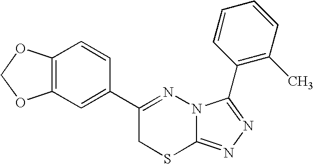 | 282 | 254 | 317 | 420 | 144 | 399 | 452 |
| N | 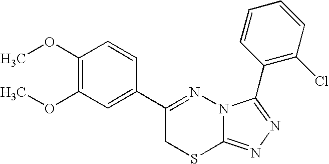 | 2398 | 367 | 559 | 755 | 278 | 502 | 278 |
| O | 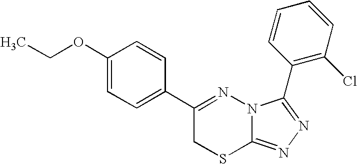 | 1335 | 129 | 279 | 1005 | ND | ND | ND |
| P | 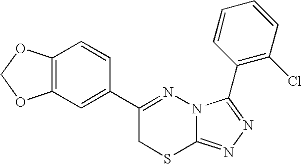 | 235 | 189 | 231 | 370 | 123 | 339 | 289 |
| Q | 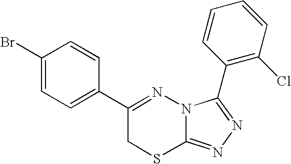 | 5346 | ND | ND | 5143 | ND | ND | ND |
| R | 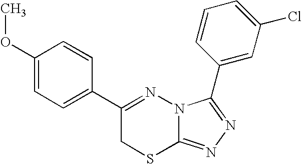 | 557 | 115 | 242 | 553 | 122 | 109 | 77 |
| S | 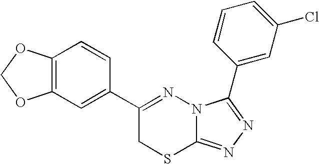 | >10000 | 1632 | >10000 | >10000 | >10000 | >1000 | >1000 |
| T | 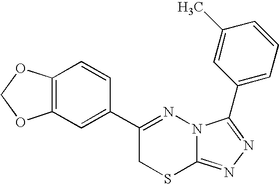 | 345 | 243 | 471 | 1229 | 261 | 221 | 262 |
| U | 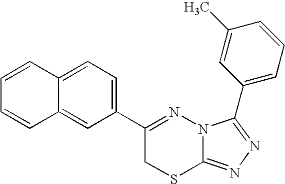 | 5545 | ND | ND | 3572 | ND | ND | ND |
| V | 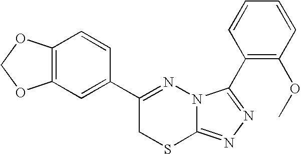 | 62 | 68 | 111 | ND | 61 | 81 | 75 |
| W | 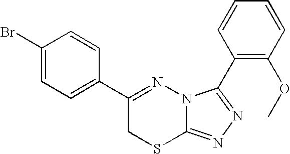 | 313 | 164 | 243 | 552 | ND | 322 | 175 |
| X | 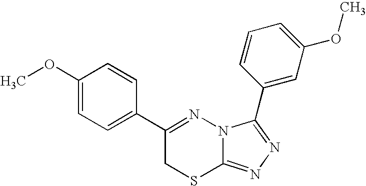 | 523 | 67 | 119 | 680 | 66 | 83 | 67 |
| Y | 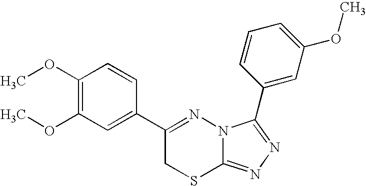 | 4130 | 1356 | 3184 | 2849 | 558 | 735 | 593 |
| Z | 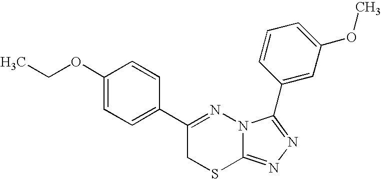 | 2811 | 331 | 693 | 2327 | ND | ND | ND |
| A1 | 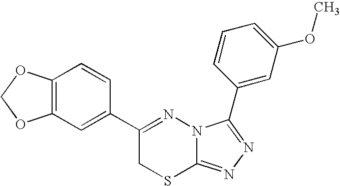 | 500 | 390 | 546 | 2492 | 426 | 507 | 447 |
| B1 | 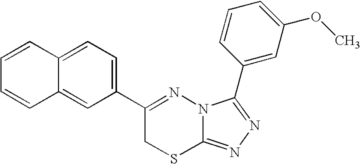 | 1416 | 930 | 1308 | 1713 | ND | ND | ND |
| C1 | 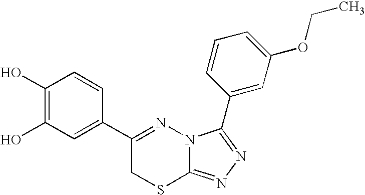 | 1316 | 1217 | >10000 | 1311 | ND | 706 | 925 |
| D1 | 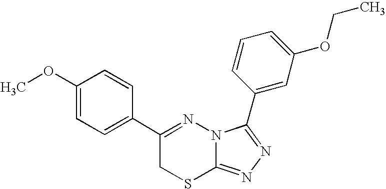 | 2300 | 778 | 1504 | 2708 | ND | ND | ND |
| E1 | 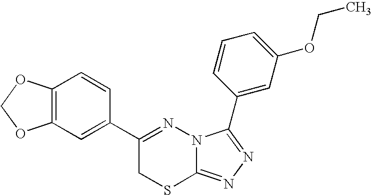 | 2119 | >10000 | >10000 | ND | >10000 | 5402 | 2589 |
| F1 | 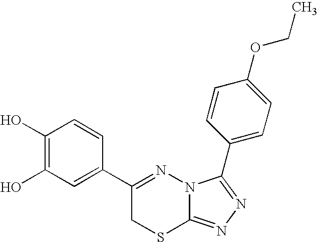 | 2576 | ND | ND | 2645 | ND | ND | ND |
| G1 | 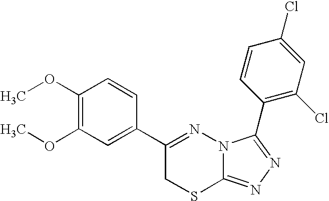 | 505 | 75 | 231 | 277 | 78 | 101 | 107 |
| H1 | 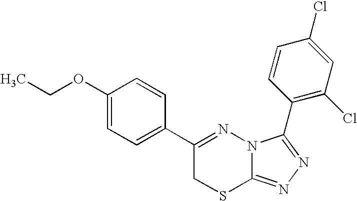 | 553 | 41 | 161 | 2012 | ND | ND | ND |
| I1 | 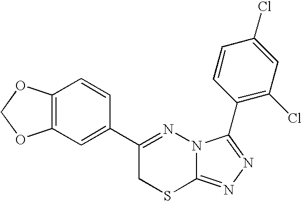 | 278 | 172 | 254 | 1382 | 221 | 171 | 150 |
| J1 | 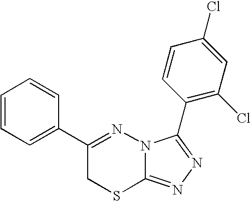 | 2871 | ND | ND | 2586 | ND | ND | ND |
| K1 | 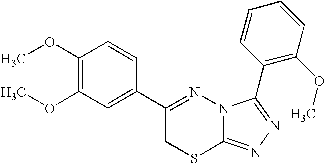 | 506 | 70 | 134 | 374 | 106 | 137 | 88 |
| L1 | 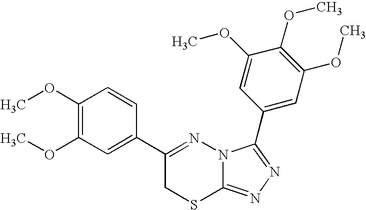 | 499 | 132 | 178 | 573 | 122 | 180 | 130 |
| M1 | 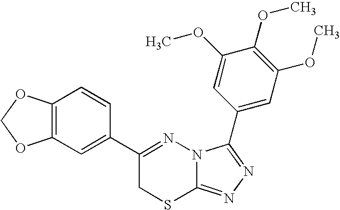 | 61 | 28 | 40 | 68 | 31 | 19 | 13 |
| N1 | 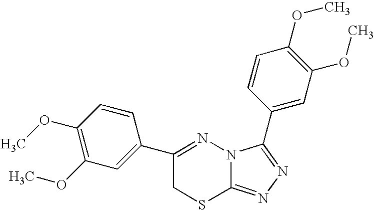 | 2220 | 1311 | 2483 | 2423 | 556 | 570 | 586 |
| O1 | 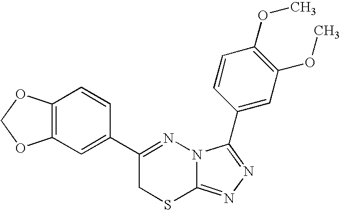 | 542 | 501 | >10000 | ND | −0.1 | 540 | 514 |
| P1 | 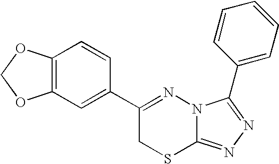 | 669 | 2641 | >10000 | ND | −0.1 | >1000 | 628 |
| Q1 | 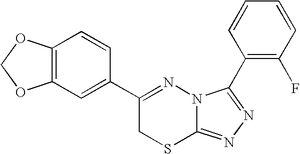 | 277 | 257 | 551 | ND | 250 | 484 | 264 |
| R1 | 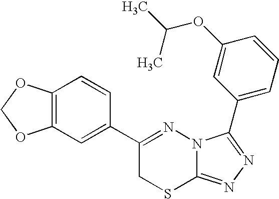 | >10000 | 5602 | >10000 | >10000 | >1000 | >1000 | >1000 |
| S1 | 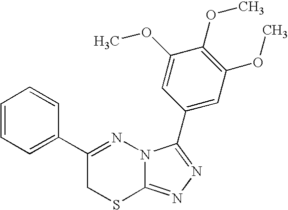 | 323 | 226 | 284 | 1258 | 313 | 619 | 316 |
| T1 | 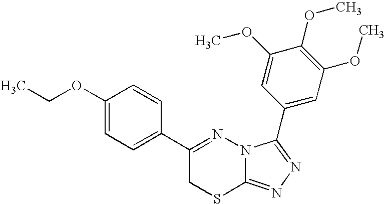 | 2583 | 519 | >10000 | 2583 | 510 | 656 | 498 |
| U1 | 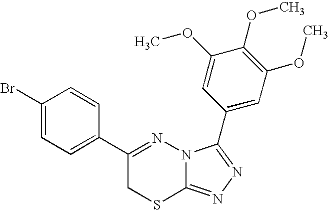 | 145 | 42 | 65 | ND | 40 | 65 | 39 |
| V1 | 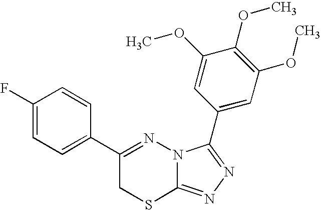 | 1317 | 1041 | >10000 | ND | 321 | 622 | 463 |
| W1 | 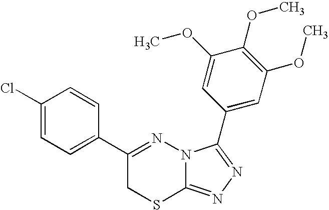 | 276 | 111 | 130 | ND | 153 | 72 | 78 |
| X1 | 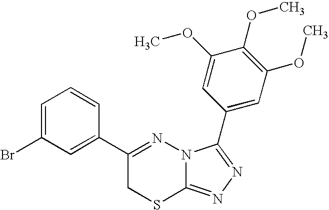 | 2635 | 2653 | 2485 | ND | 2507 | 3410 | 2513 |
| Y1 | 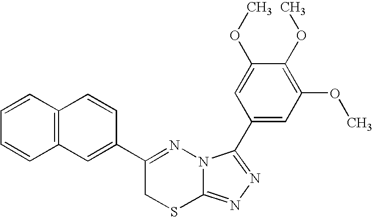 | 556 | 81 | 119 | ND | 139 | 127 | 99 |
| Z1 | 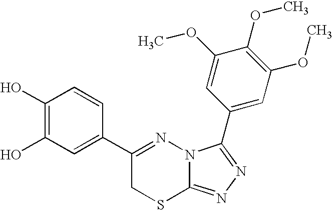 | 284 | 101 | 240 | ND | 200 | 176 | 219 |
| A2 | 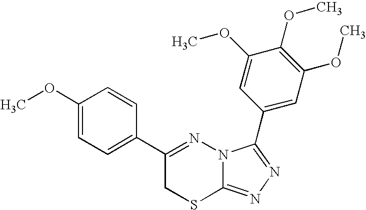 | 517 | 36 | 154 | ND | 36 | 49 | 32 |
| B2 | 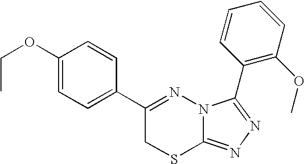 | 175 | 30 | 40 | 106 | 106 | ND | ND |
| C2 | 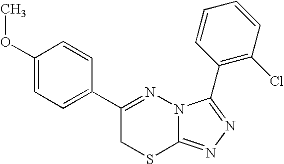 | 395 | 60 | 177 | ND | ND | ND | ND |
| D2 | 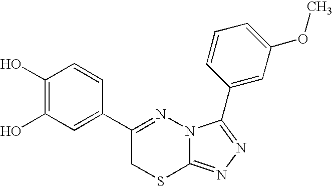 | 687 | 112 | 525 | ND | ND | ND | ND |
| E2 | 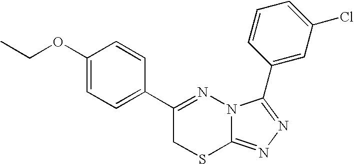 | 1102 | 429 | 681 | ND | ND | ND | ND |
| F2 | 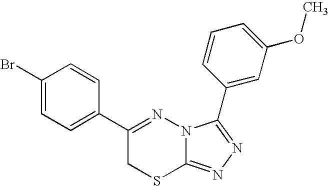 | 5398 | 2705 | >10000 | ND | ND | ND | ND |
| G2 | 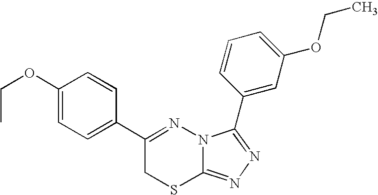 | 6374 | 2547 | >10000 | ND | ND | ND | ND |
| H2 | 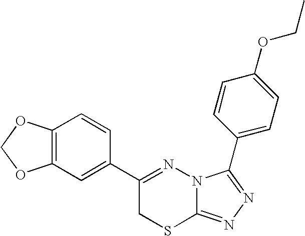 | >1000 | >1000 | >1000 | >1000 | >1000 | >1000 | 542 |
| I2 | 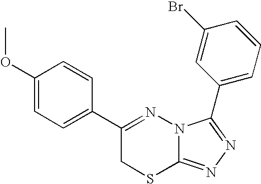 | 905 | 329 | 612 | ND | 629 | 523 | 379 |
| J2 | 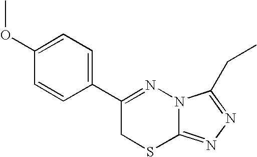 | 5628 | 5083 | >10000 | ND | >10000 | 5557 | 5425 |
| K2 | 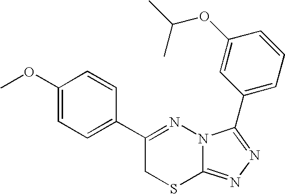 | 5472 | 5564 | >10000 | ND | 5533 | 5474 | 5845 |
| L2 | 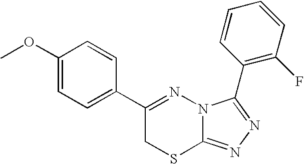 | 553 | 55 | 122 | ND | 76 | 85 | 60 |
| M2 | 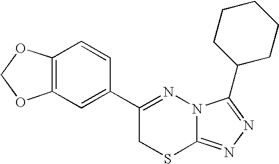 | 4331 | 4767 | >10000 | ND | 5239 | 5187 | 5149 |
| N2 | 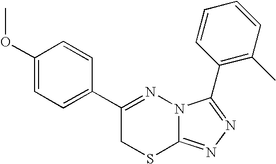 | 191 | 31 | 34 | ND | 41 | 53 | 29 |
| O2 | 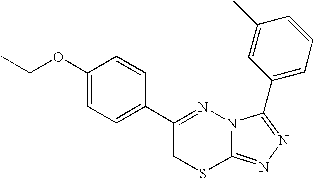 | 1422 | 433 | 665 | ND | 635 | 641 | 597 |
| P2 | 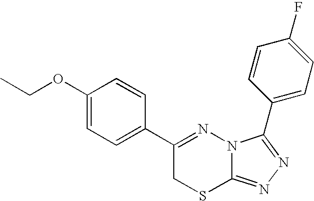 | 2158 | 473 | 1471 | ND | 880 | 901 | 660 |
| Q2 | 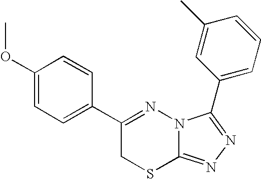 | 503 | 61 | 100 | ND | 80 | 121 | 65 |
| R2 | 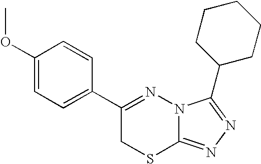 | 2442 | 699 | 2277 | ND | 1273 | 1456 | 1198 |
| S2 | 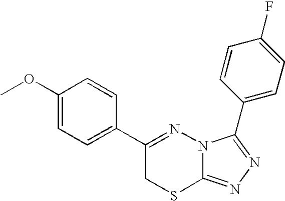 | 1657 | 271 | 700 | ND | 546 | 569 | 339 |
| T2 | 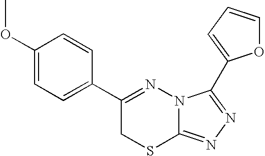 | 5413 | 5071 | >10000 | ND | 5328 | 5350 | 5044 |
| U2 | 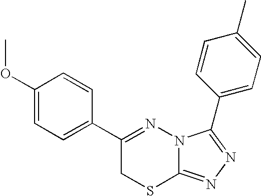 | 537 | 86 | 168 | ND | 129 | 190 | 127 |
| V2 | 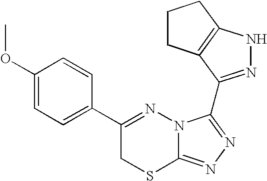 | 312 | 195 | 268 | ND | 250 | 444 | 256 |
| W2 | 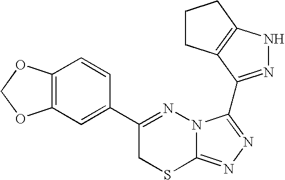 | 431 | 506 | 599 | ND | 592 | 710 | 523 |
| X2 | 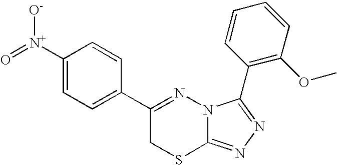 | ND | ND | ND | ND | 2575 | 4857 | 2580 |
| Y2 | 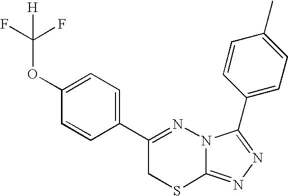 | ND | ND | ND | ND | 2338 | 2939 | 2082 |
| Z2 | 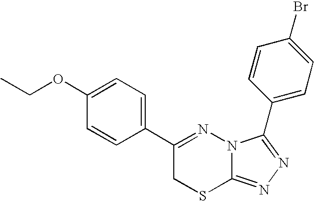 | 349 | 179 | 395 | ND | 158 | 229 | 149 |
| A3 | 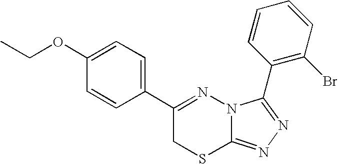 | 1083 | 243 | 314 | ND | 104 | 268 | 125 |
| B3 | 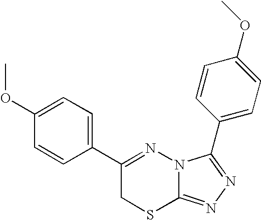 | 1385 | 604 | 1038 | ND | 381 | 763 | 372 |
| C3 | 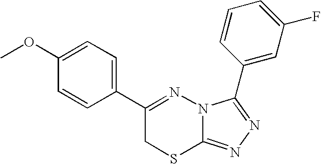 | 1713 | 126 | 424 | ND | 132 | 247 | 152 |
| D3 | 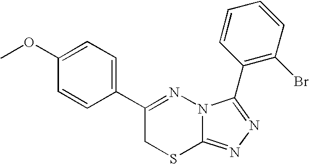 | 332 | 61 | 143 | ND | 54 | 115 | 76 |
| E3 | 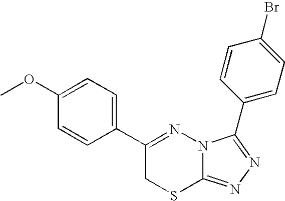 | 244 | 85 | 157 | ND | 48 | 125 | 77 |
| F3 | 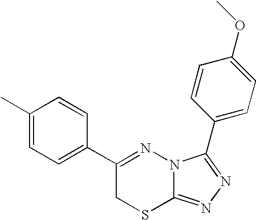 | ND | ND | ND | ND | 446 | 688 | 336 |
| G3 | 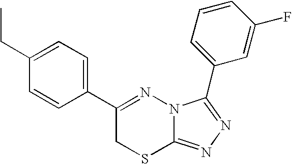 | ND | ND | ND | ND | 1018 | 1706 | 1226 |
| H3 | 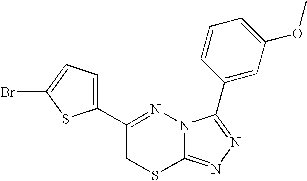 | ND | ND | ND | ND | >10000 | >10000 | 5301 |
| I3 | 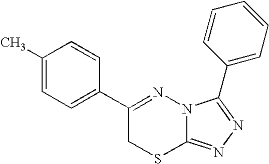 | ND | ND | ND | ND | 633 | 1150 | 642 |
| J3 | 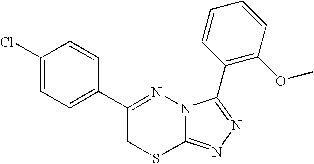 | 599 | 525 | 648 | ND | 332 | ND | ND |
| K3 | 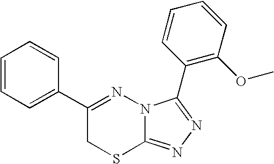 | 3653 | 3781 | 5333 | ND | 2833 | ND | ND |
| L3 | 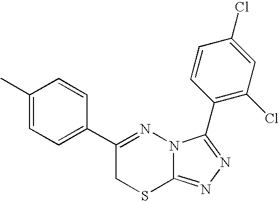 | 286 | 107 | 134 | ND | 82 | ND | ND |
| M3 | 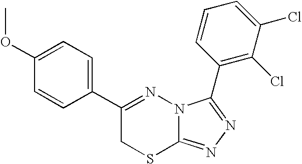 | 268 | 82 | 124 | ND | 68 | ND | ND |
| N3 | 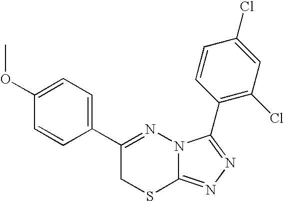 | 284 | 52 | 55 | ND | 32 | ND | ND |
| O3 | 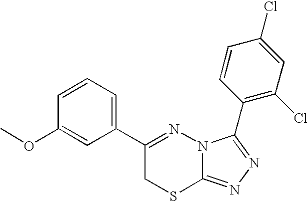 | 1295 | 765 | 2618 | ND | 585 | ND | ND |
| P3 | 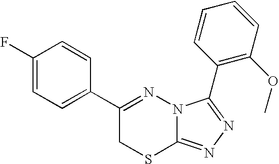 | 2539 | 1978 | 4488 | ND | 1863 | ND | ND |
| Q3 | 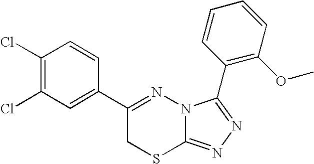 | >10000 | >10000 | >10000 | ND | 292 | ND | ND |
| 2 | 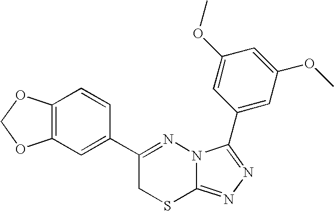 | 508 | 141 | 139 | ND | 117 | 112 | 89 |
| 4 | 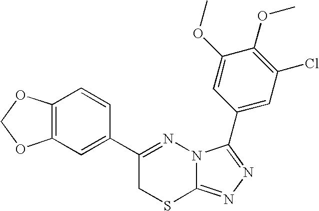 | 168 | 47 | 61 | ND | 36 | 32 | 31 |
| 5 | 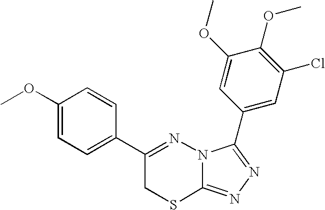 | ND | ND | ND | ND | 71 | 133 | 78 |
| 6 | 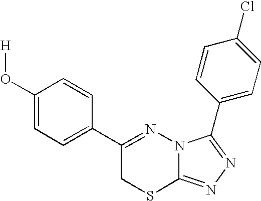 | 855 | 5429 | >10000 | ND | >10000 | >10000 | 7651 |
| 7 | 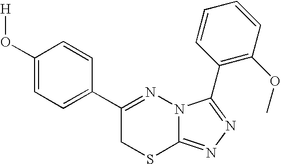 | 173 | 148 | 280 | ND | 253 | 478 | 262 |
| 8 | 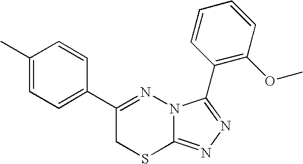 | 146 | 25 | 60 | ND | 31 | 57 | 30 |
| 9 | 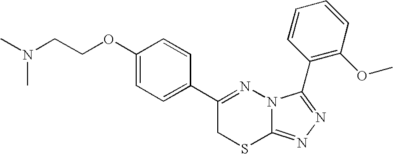 | ND | ND | ND | ND | 2558 | 3373 | 2654 |
| 10 | 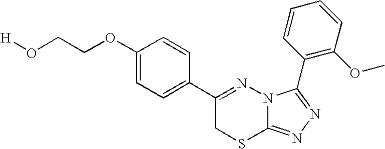 | 300 | 267 | 612 | ND | 254 | 431 | 306 |
| 11 | 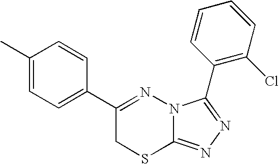 | ND | ND | ND | ND | 71 | 157 | 72 |
| 12 | 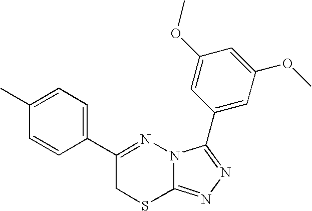 | 760 | 61 | 71 | ND | 20 | 55 | 20 |
| 13 | 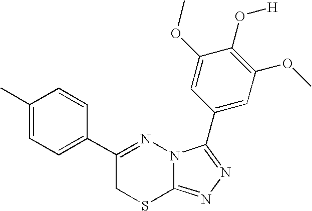 | 625 | 101 | 189 | ND | 73 | 52 | 69 |
| 14 | 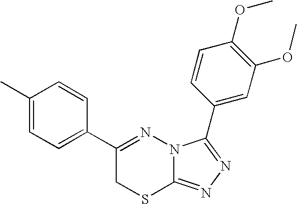 | 396 | 65 | 107 | ND | 131 | 198 | 125 |
| 15 | 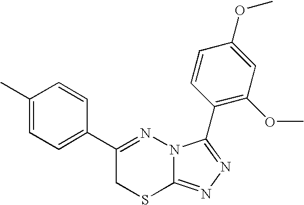 | 34 | 5 | 8 | ND | 7 | 12 | 8 |
| 16 | 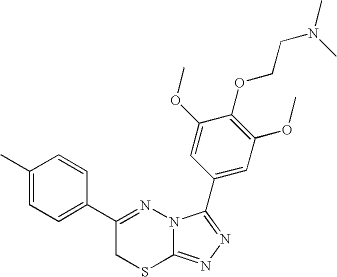 | 609 | ND | ND | ND | ND | ND | ND |
| 17 | 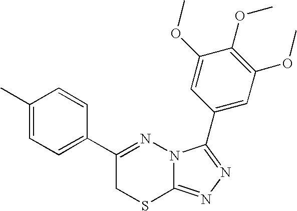 | 118 | 24 | 46 | ND | 29 | 50 | 31 |
| 18 | 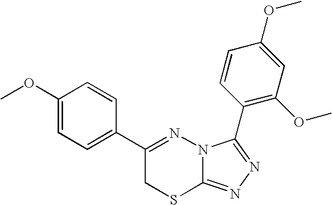 | 32 | 6 | 17 | ND | 7 | 15 | 7 |
| 19 | 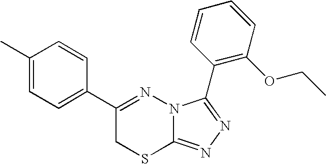 | 35 | 24 | 32 | ND | 17 | 29 | 20 |
| 20 | 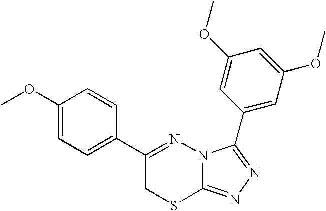 | ND | ND | ND | ND | 55 | 95 | 36 |
| 21 | 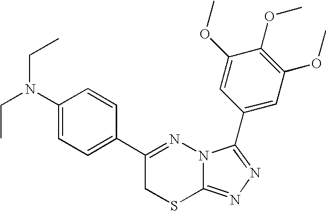 | ND | ND | ND | ND | 280 | 434 | 272 |
| 22 | 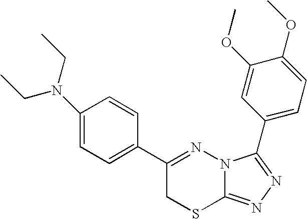 | 534 | 122 | 188 | ND | 214 | 270 | 258 |
| 23 | 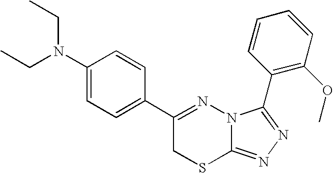 | 32 | 18 | 26 | ND | 15 | 27 | 27 |
| 24 | 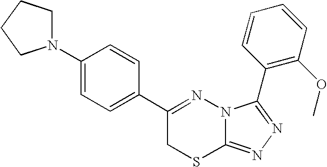 | 91 | 41 | 62 | ND | 35 | 107 | 62 |
| 25 | 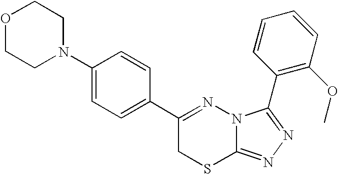 | 612 | 694 | 1264 | ND | 321 | 511 | 275 |
| 26 | 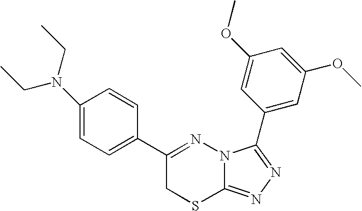 | 297 | 114 | 280 | ND | 106 | 154 | 73 |
| 27 | 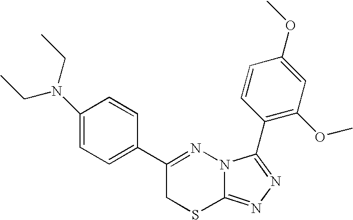 | 44 | 47 | 55 | ND | 31 | 46 | 55 |
| 28 | 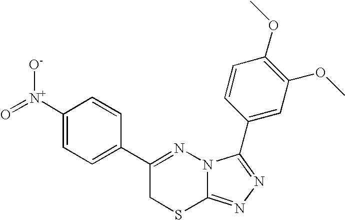 | >10000 | >10000 | >10000 | ND | >10000 | >10000 | >10000 |
| 29 | 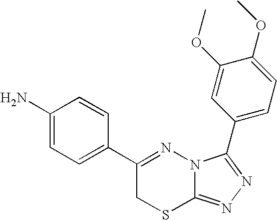 | 4721 | 2544 | 5280 | ND | ND | ND | 545 |
| 30 | 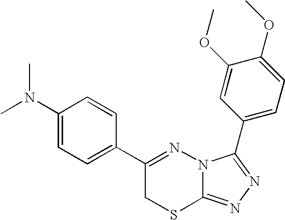 | 360 | 119 | 221 | ND | 55 | 64 | 57 |
| 31 | 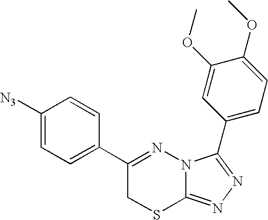 | 2392 | 619 | 5554 | ND | 544 | 534 | 520 |
| 32 | 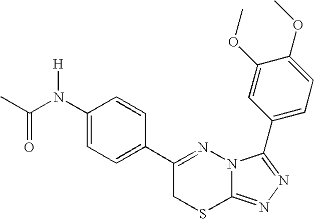 | ND | ND | ND | ND | >10000 | >10000 | 4861 |
| 33 | 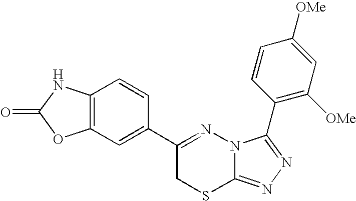 | ND | ND | ND | ND | 2728 | 2405 | 2680 |
| 34 | 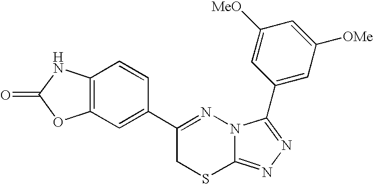 | 4605 | 4923 | 5090 | ND | 4934 | ND | ND |
| 35 | 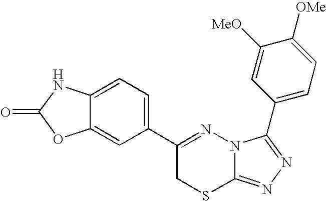 | >10,000 | >10,000 | >10,000 | ND | >10,000 | ND | ND |
| 36 | 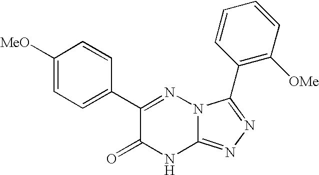 | >10,000 | ND | >10,000 | ND | >10,000 | ND | ND |
| 37 | 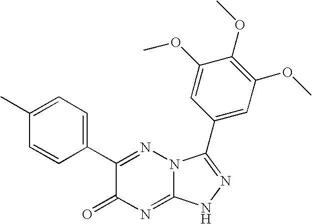 | 5829 | 5042 | >10000 | ND | >10000 | ND | ND |
| 38 | 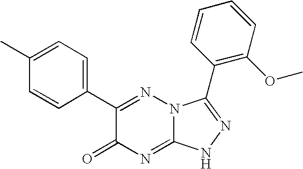 | >10000 | >10000 | >10000 | ND | >10000 | ND | ND |
| 39 | 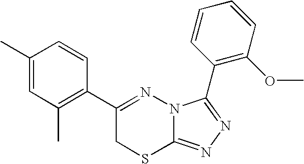 | 2697 | 943 | 2579 | ND | 1275 | ND | ND |
| 40 | 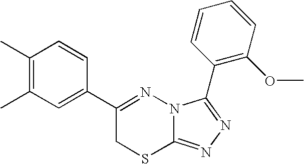 | 163 | 41 | 65 | ND | 49 | ND | ND |
| 41 | 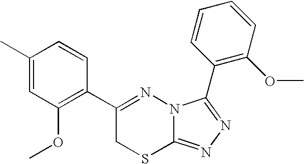 | 422 | 180 | 289 | ND | 182 | ND | ND |
| 42 | 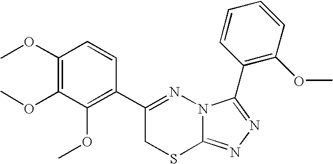 | 335 | 488 | 753 | ND | 316 | ND | ND |
| 43 | 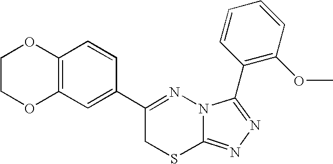 | 35 | 17 | 25 | ND | 15 | ND | ND |
| 44 | 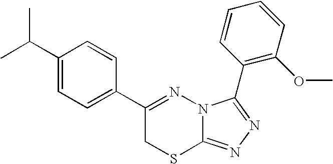 | 281 | 243 | 275 | ND | 248 | ND | ND |
| 45 | 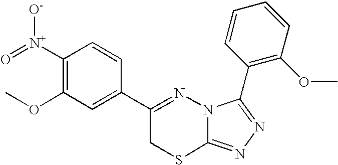 | >10000 | >10000 | >10000 | ND | >10000 | ND | ND |
| 46 | 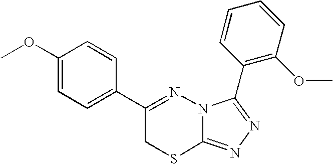 | 43 | 17 | 19 | ND | 11 | ND | ND |
| 47 | 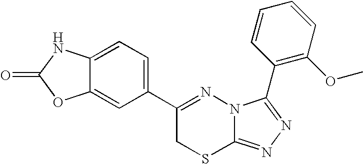 | 2331 | 2745 | 5544 | ND | 4793 | ND | ND |
| 48 | 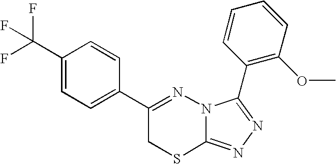 | 545 | 511 | 569 | ND | 480 | ND | ND |
| 49 | 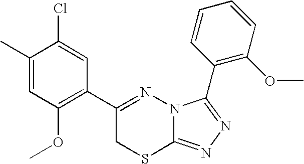 | >10000 | 5654 | >10000 | ND | 5587 | ND | ND |
| 50 | 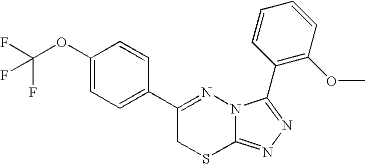 | 549 | 530 | 564 | ND | 500 | ND | ND |
| 51 | 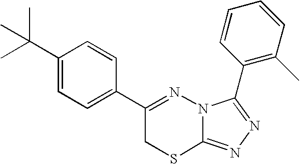 | >10000 | >10000 | >10000 | ND | >10000 | ND | ND |
| 52 | 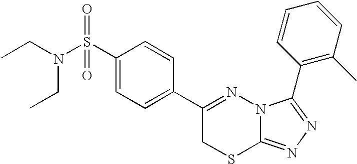 | >10000 | >10000 | >10000 | ND | >10000 | ND | ND |
| 53 | 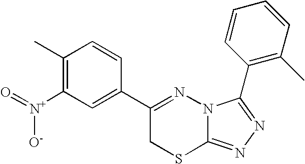 | >10000 | >10000 | >10000 | ND | >10000 | ND | ND |
| 54 | 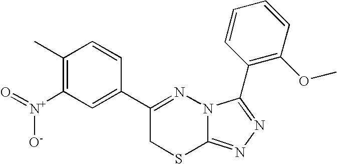 | 554 | 306 | 441 | ND | 290 | ND | ND |
| 55 | 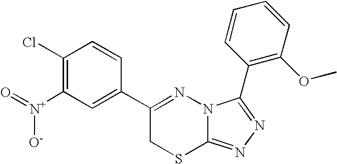 | 2040 | 1870 | >10000 | ND | >10000 | ND | ND |
| 56 | 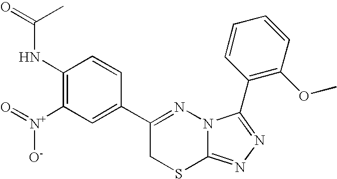 | 476 | 328 | 506 | ND | 270 | ND | ND |
| 57 | 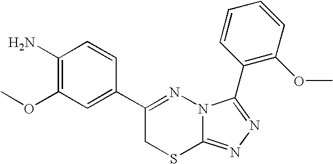 | 2639 | 2284 | 2872 | ND | 2330 | ND | ND |
| 58 | 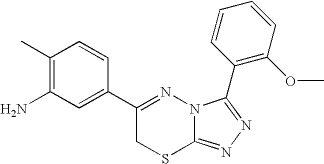 | 76 | 32 | 54 | ND | 30 | ND | ND |
| 59 | 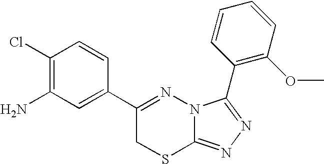 | 102 | 43 | 59 | ND | 34 | ND | ND |
| 60 | 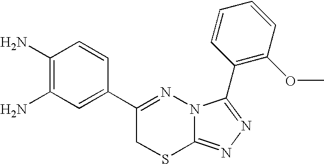 | >10000 | >10000 | >10000 | ND | >10000 | ND | ND |
| 61 | 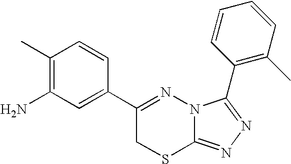 | 330 | 109 | 137 | ND | 71 | ND | ND |
| 62 | 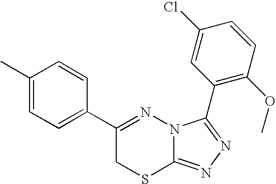 | 101 | 24 | 31 | ND | 18 | ND | ND |
| 63 | 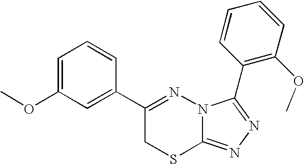 | 261 | 146 | 277 | ND | 128 | ND | ND |
| 64 | 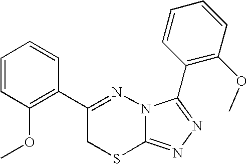 | >10000 | >10000 | >10000 | ND | >10000 | ND | ND |
| 65 | 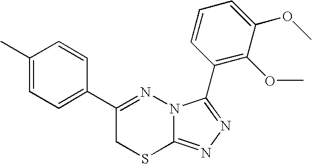 | 610 | 522 | 635 | ND | 561 | ND | ND |
| 66 | 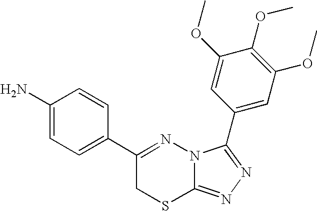 | 72 | 21 | 29 | ND | 16 | ND | ND |
| 67 | 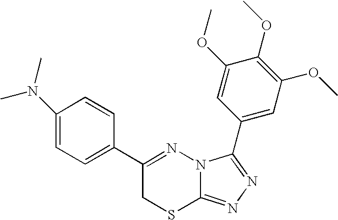 | 175 | 43 | 58 | ND | 61 | ND | ND |
| 68 | 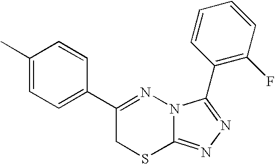 | 5257 | 636 | 1259 | ND | 481 | ND | ND |
| 69 | 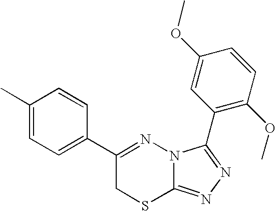 | 42 | 15 | 17 | ND | 11 | ND | ND |
| 70 | 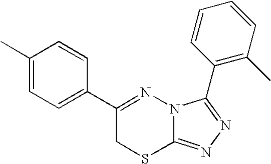 | 546 | 165 | 205 | ND | 157 | ND | ND |
| 71 | 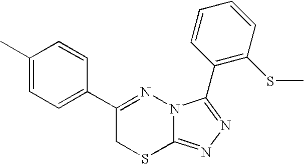 | 453 | 70 | 76 | ND | 65 | ND | ND |
| 72 | 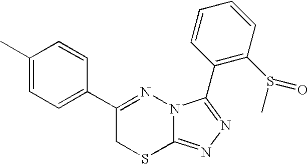 | >10000 | >10000 | >10000 | ND | >10000 | ND | ND |
| 73 | 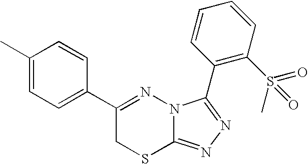 | >10000 | >1000 | >10000 | ND | >10000 | ND | ND |
| 74 | 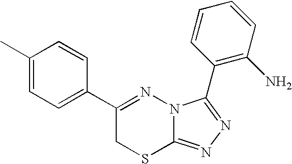 | >10000 | 529 | 557 | ND | 507 | ND | ND |
| 75 | 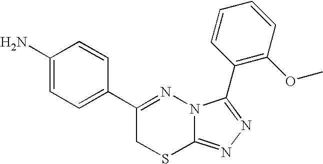 | 238 | 203 | 266 | ND | 136 | ND | ND |
| 76 | 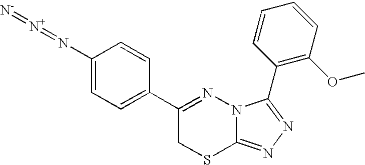 | 93 | 54 | 72 | ND | 62 | ND | ND |
| 77 | 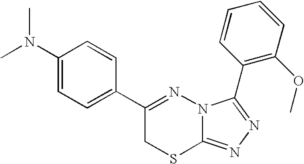 | 18 | 14 | 11 | ND | 7 | ND | ND |
| 78 | 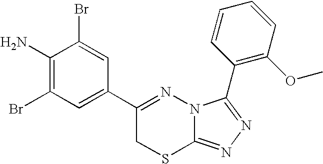 | 259 | 51 | 124 | ND | 73 | ND | ND |
| 79 | 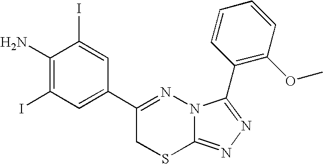 | 907 | 562 | 1250 | ND | 304 | ND | ND |
| 80 | 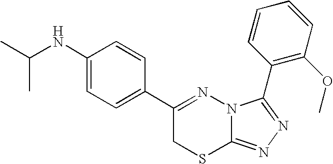 | 188 | 117 | 204 | ND | 115 | ND | ND |
| 81 | 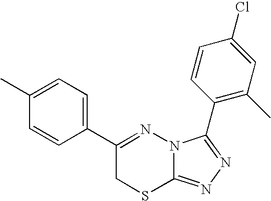 | 270 | 39 | 67 | ND | 55 | ND | ND |
| 82 | 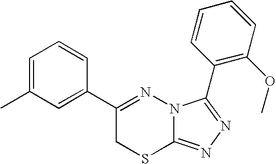 | 605 | 500 | 370 | ND | 497 | ND | ND |
| 83 | 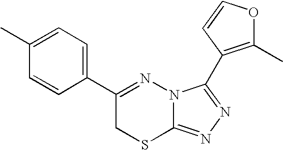 | 2543 | 523 | 683 | ND | 332 | ND | ND |
| 84 | 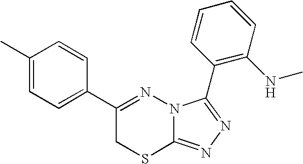 | 528 | 148 | 130 | ND | 125 | ND | ND |
| 85 | 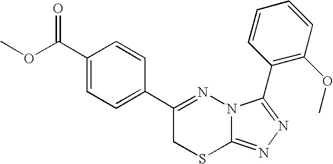 | 274 | 253 | 298 | ND | 209 | ND | ND |
| 86 | 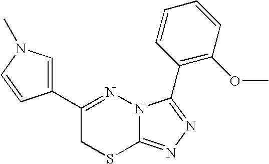 | 525 | 380 | 1320 | ND | 320 | ND | ND |
| 87 | 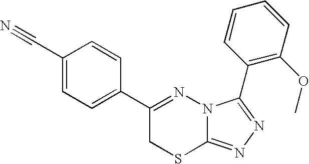 | 3327 | 2553 | 5091 | ND | 2280 | ND | ND |
| 88 | 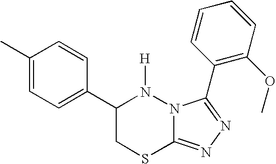 | 5326 | 2527 | 2803 | ND | 1534 | ND | ND |
| 89 | 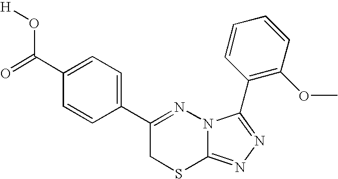 | >10000 | >10000 | >10000 | ND | >10000 | ND | ND |
| 90 | 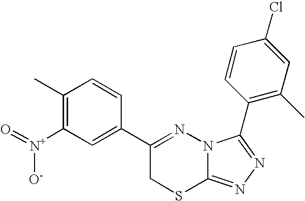 | 915 | 412 | 492 | ND | 303 | ND | ND |
| 91 | 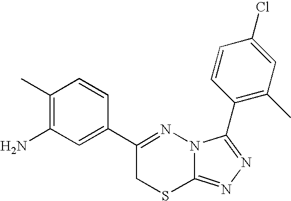 | 63 | 28 | 30 | ND | 16 | ND | ND |
| 92 | 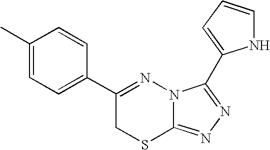 | >10000 | >10000 | >10000 | ND | >10000 | ND | ND |
| 93 | 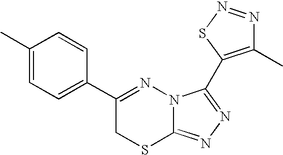 | >10000 | >10000 | >10000 | ND | >10000 | ND | ND |
| 94 | 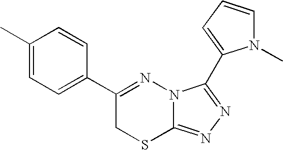 | 2630 | 1441 | 5512 | ND | 1286 | ND | ND |
| 95 | 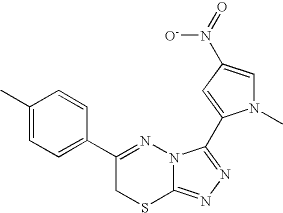 | 2698 | 478 | 693 | ND | 328 | ND | ND |
| 96 | 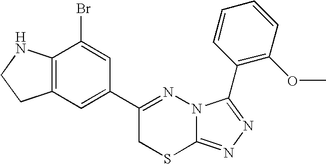 | 136 | 35 | 79 | ND | 31 | ND | ND |
| 97 | 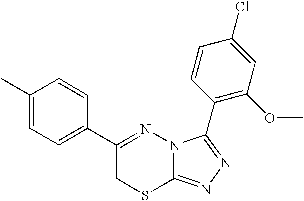 | 34 | 6 | 8 | ND | 4 | ND | ND |
| 98 | 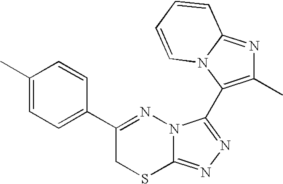 | 166 | 59 | 98 | ND | 66 | ND | ND |
| 99 | 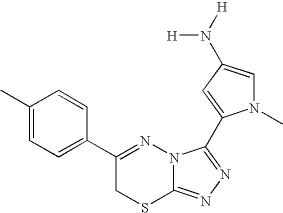 | 5421 | 1264 | 1781 | ND | 1182 | ND | ND |

ND: Not determined

Thus, 3-(2-chlorophenyl)-6-(3,4-methylenedioxyphenyl)-7H-[1,2,4]triazolo[3,4-b][1,3,4]thiadiazine (Example P) and analogs are identified as potent caspase cascade activators and inducers of apoptosis in several solid tumor cells. Importantly, these compounds are active in human Burkitt's lymphoma cell line Namalwa, human lymphoma cell line Raji, and human B cell lymphoblastoid cell line Ramos, three cell lines that are known to have deregulated cMyc.

### EXAMPLE 101

### Identification of 3-(2-Chlorophenyl)-6-(3,4-methylenedioxyphenyl)-7H-[1,2,4]triazolo[3,4-b][1,3,4]thiadiazine and Analogs as Antineoplastic Compound that Inhibits Cell Proliferation (GI₅₀)

Human breast cancer cell lines T-47D, human hepatocellular carcinoma cell line SNU398, human colon carcinoma cell line HCT116, human lung cancer cell line H1299, human leukemia cell line K562, human lymphoma cell line Raji, human B cell lymphoblastoid cell line Ramos, and human Burkitt's lymphoma cell line Namalwa were grown and harvested as in Example 100. An aliquot of 90 μL of cells (4.4×10⁴ cells/mL) was added to a well of a 96-well microtiter plate containing 5 μL of a 10% DMSO in RPMI-1640 media solution containing 10 nM to 100 μM of 3-(2-chlorophenyl)-6-(3,4-methylenedioxyphenyl)-7H-[1,2,4]triazolo[3,4-b][1,3,4]thiadiazine (1 nM to 10 μM final). An aliquot of 45 μL of cells was added to a well of a 96-well microtiter plate containing 5 μL of a 10% DMSO in RPMI-1640 media solution without compound as the control sample for maximal cell proliferation (L_{Max}). The samples were mixed by agitation and then incubated at 37° C. for 48 h in a 5% CO₂-95% humidity incubator. After incubation, the samples were removed from the incubator and 25 μL of CellTiter-Glo™ reagent (Promega) was added. The samples were mixed by agitation and incubated at room temperature for 10-15 min. Plates were then read using a luminescent plate reader (Model SPECTRAfluor Plus, Tecan) to give Lₜₑₛₜ values.

Baseline for GI₅₀ (dose for 50% inhibition of cell proliferation) of initial cell numbers was determined by adding an aliquot of 45 μL of cells or 45 μL of media, respectively, to wells of a 96-well microtiter plate containing 5 μL of a 10% DMSO in RPMI-1640 media solution. The samples were mixed by agitation and then incubated at 37° C. for 0.5 h in a 5% CO₂-95% humidity incubator. After incubation, the samples were removed from the incubator and 25 μL of CellTiter-Glo™ reagent (Promega) was added. The samples were mixed by agitation and incubated at 37° C. for 10-15 min at room temperature in a 5% CO₂-95% humidity incubator. Fluorescence was read as above, (L_{Start}) defining luminescence for initial cell number used as baseline in GI₅₀ determinations.

Calculation:
GI₅₀ (dose for 50% inhibition of cell proliferation) is the concentration where [(Lₜₑₛₜ−L_{Start})/(L_{Max}−L_{Start})]=0.5.

The GI₅₀ (nM) are summarized in Table 11:

TABLE II
GI₅₀ in Cancer Cells

| | GI₅₀ (nM) | | | | | | | |
|---|---|---|---|---|---|---|---|---|
| Example | T47D | SNU 398 | HCT 116 | H1299 | K562 | Raji | Ramos | Namalwa |
| B | ND | ND | 396 | ND | 267 | 133 | 108 | 254 |
| D | 144 | ND | 2115 | 42 | 31 | 1498 | 21 | 1503 |
| F | 151 | ND | 419 | 58 | 47 | 136 | 15 | 120 |
| G | 97 | ND | ND | 307 | ND | ND | ND | ND |
| K | 2624 | ND | ND | 667 | ND | ND | ND | ND |
| L | ND | ND | 141 | ND | 27 | 62 | 17 | 24 |
| M | 124 | ND | 825 | 214 | 239 | 2216 | 164 | 122 |
| N | 2538 | ND | ND | 510 | ND | ND | ND | ND |
| P | 175 | ND | 884 | 202 | 214 | 426 | 118 | 472 |
| R | ND | ND | 121 | ND | 42 | 89 | 17 | 117 |
| T | 375 | ND | ND | 314 | ND | ND | ND | ND |
| W | 135 | ND | 489 | 161 | 178 | 299 | 141 | 314 |
| X | 400 | 55 | 157 | ND | 32 | 62 | 14 | 119 |
| C1 | 1369 | ND | 2343 | 1014 | 453 | 2890 | 1392 | 2867 |
| E1 | 5000 | 4604 | 5000 | ND | ND | ND | ND | ND |
| G1 | 130 | 60 | 81 | 48 | 32 | 90 | 45 | 13 |
| K1 | 461 | ND | 253 | 191 | 99 | 39 | 41 | 38 |
| L1 | 200 | 147 | 200 | 339 | 163 | 150 | 76 | 243 |
| M1 | 22 | 12 | 25 | 166 | 18 | 41 | 16 | 13 |
| N1 | 2646 | ND | ND | 1936 | ND | ND | ND | ND |
| I2 | ND | ND | 376 | ND | 96 | 176 | 119 | 400 |
| L2 | ND | ND | 142 | ND | 34 | 247 | 20 | 68 |
| N2 | ND | ND | 171 | ND | 38 | 164 | 13 | 34 |
| Q2 | ND | ND | 359 | ND | 37 | 587 | 33 | 112 |
| U2 | ND | ND | 1103 | ND | 72 | 730 | 100 | 256 |
| V2 | ND | ND | 1129 | ND | 400 | 1262 | 346 | 975 |
| W2 | 1496 | 294 | 5901 | ND | 1871 | 3000 | 1289 | 2549 |
| Z2 | ND | ND | 349 | ND | 45 | 114 | 50 | 12 |
| A3 | ND | ND | 361 | ND | 41 | 103 | 17 | 79 |
| B3 | ND | ND | 1152 | ND | 71 | 377 | 321 | 683 |
| C3 | ND | ND | 852 | ND | 63 | 130 | 100 | 112 |
| D3 | ND | ND | 100 | ND | 44 | 85 | 49 | 33 |
| E3 | ND | ND | 200 | ND | 33 | 66 | 21 | 47 |
| N3 | 34 | 13 | 40 | ND | ND | ND | ND | 64 |
| 2 | ND | ND | 190 | ND | 27 | 53 | 11 | 20 |
| 4 | ND | ND | 150 | ND | 24 | 26 | 12 | 31 |
| 7 | ND | ND | 1177 | ND | 219 | 1000 | 176 | 579 |
| 8 | 17 | 150 | 15 | ND | 43 | 27 | 16 | 66 |
| 10 | ND | ND | 1673 | ND | 315 | 746 | 173 | 618 |
| 12 | ND | ND | 50 | ND | 7 | 15 | 5 | 6 |
| 13 | ND | ND | 165 | ND | 51 | 97 | 50 | 18 |
| 14 | ND | ND | 1011 | ND | 87 | 838 | 300 | 137 |
| 15 | ND | ND | 10 | ND | 4 | 4 | 1 | 6 |
| 16 | 936 | 2000 | 1154 | ND | ND | ND | ND | ND |
| 17 | 15 | 25 | 21 | ND | ND | ND | ND | ND |
| 18 | 28 | 1 | 34 | ND | ND | ND | ND | 6 |
| 19 | 48 | 12 | 67 | ND | ND | ND | ND | 12 |
| 20 | 373 | 11 | 99 | ND | ND | ND | ND | 13 |
| 21 | 185 | 20 | 680 | ND | ND | ND | ND | 176 |
| 23 | ND | ND | 13 | ND | 2 | 2 | 3 | 7 |
| 24 | ND | ND | 41 | ND | 17 | 15 | 16 | 64 |
| 25 | ND | ND | 1984 | ND | 317 | 285 | 105 | 161 |
| 26 | ND | ND | 20 | ND | 20 | 17 | 20 | 16 |
| 27 | ND | ND | 23 | ND | 10 | 3 | 1 | 2 |
| 30 | ND | ND | 126 | ND | 50 | 157 | 100 | 102 |
| 31 | ND | ND | 4254 | ND | 581 | 6328 | 503 | 1468 |
| 40 | 137 | 50 | 129 | ND | ND | ND | ND | 29 |
| 41 | 261 | 117 | 1269 | ND | ND | ND | ND | 208 |
| 42 | 288 | 121 | 482 | ND | ND | ND | ND | 433 |
| 43 | 34 | 13 | 40 | ND | ND | ND | ND | 64 |
| 46 | 12 | 10 | 8 | ND | ND | ND | ND | 13 |
| 47 | 3022 | 1221 | 3000 | ND | ND | ND | ND | 1475 |
| 48 | 690 | 120 | 324 | ND | ND | ND | ND | 452 |
| 58 | 26 | 24 | 52 | ND | ND | ND | ND | 25 |
| 59 | 103 | 98 | 149 | ND | ND | ND | ND | 84 |
| 61 | 269 | 228 | 400 | ND | ND | ND | ND | 150 |
| 62 | 33 | 10 | 50 | ND | ND | ND | ND | 15 |
| 63 | 371 | 119 | 300 | ND | ND | ND | ND | 232 |
| 65 | 1512 | 204 | 4679 | ND | ND | ND | ND | 1227 |
| 66 | 47 | 24 | 127 | ND | ND | ND | ND | 43 |
| 68 | 2755 | 144 | 947 | ND | ND | ND | ND | 269 |
| 69 | 50 | 4 | 41 | ND | ND | ND | ND | 10 |
| 70 | 500 | 144 | 1010 | ND | ND | ND | ND | 158 |
| 71 | 276 | 91 | 169 | ND | ND | ND | ND | 77 |
| 75 | 345 | 180 | 867 | ND | ND | ND | ND | 179 |
| 76 | 34 | 23 | 109 | ND | ND | ND | ND | 55 |
| 77 | 5 | 2 | 12 | ND | ND | ND | ND | 5 |
| 80 | 297 | 115 | 300 | ND | ND | ND | ND | 170 |
| 81 | 255 | 21 | 79 | ND | ND | ND | ND | 18 |
| 84 | 498 | 154 | 911 | ND | ND | ND | ND | 150 |

ND, Not determined.

Thus, 3-(2-chlorophenyl)-6-(3,4-methylenedioxyphenyl)-7H-[1,2,4]triazolo[3,4-b][1,3,4]thiadiazine (Example P) and analogs are identified as antineoplastic compound that inhibits cell proliferation in several solid tumor cell lines.

Several compounds were tested in human sarcoma cell line MES-SA and multi-drug resistant (MDR) human sarcoma cell line MES-SA/ADR, murine leukemia cell line P388 and multi-drug resistant (MDR) murine leukemia cell line P388/ADR, and the data are summarized in Table III.

TABLE III
GI₅₀ of Selective Compounds in Cancer Cells

GI₅₀ (nM)

| | | MES-SA/ | | |
|---|---|---|---|---|
| Example | MES-SA | ADR | P388 | P388/ADR |
| V | 83 | 123 | 31 | 43 |
| X | 418 | 196 | 57 | 77 |
| M1 | 17 | 25 | 7 | 10 |
| A2 | 44 | 63 | 23 | 25 |
| 2 | 59 | 154 | 20 | 29 |
| 4 | 30 | 28 | 12 | 16 |

Thus, 3-(3-methoxyphenyl)-6-(4-methoxyphenyl)-7H-[1,2,4]triazolo[3,4-b][1,3,4]thiadiazine (Example X) and analogs are identified as antineoplastic compound that have similar activity against MES-SA and its corresponding multi-drug resistant cell MES-SA/ADR, as well as P388 and its corresponding multi-drug resistant cell P388/ADR, suggesting that these compounds should be useful for the treatment of drug resistant cancers.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

1. A method of treating a disorder responsive to the induction of apoptosis in an animal suffering therefrom, comprising administering to an animal in need of such treatment an effective amount of a compound having the Formula I: 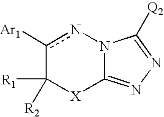
or pharmaceutically acceptable salts or prodrugs or tautomers thereof, wherein:
the dashed line signifies an optional double bond;
Ar₁ is an optionally substituted aryl or optionally substituted heteroaryl;
Q₂ is an optionally substituted alkyl, carbocyclic, heterocyclic, aryl or heteroaryl;
R₁ and R₂ independently are hydrogen, halo, optionally substituted amino, optionally substituted alkoxy, optionally substituted C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, hydroxyalkoxy, amino, aminoalkyl, aminoalkoxy, carboxyalkyl, nitro, cyano, acylamido, hydroxy, thiol, acyloxy, azido, carboxy, carbonylamido or optionally substituted alkylthiol; or R₁ and R₂ are combined as ═O; and
X is S, O or NR₃, wherein R₃ is hydrogen or an optionally substituted alkyl or aryl.

2-8. (canceled)

9. A method of treating a disorder responsive to the induction of apoptosis in an animal suffering therefrom, comprising administering to an animal in need of such treatment an effective amount of a compound having the Formula II: 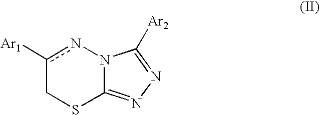
or a pharmaceutically acceptable salt or prodrug or tautomer thereof, wherein:
the dashed line signifies an optional double bond;
Ar₁ and Ar₂ independently are optionally substituted aryl or optionally substituted heteroaryl.

10. The method of claim 9, wherein the dashed line is a double bond.

11. The method of claim 9, wherein Ar₁ and Ar₂ independently are phenyl, naphthyl, pyridyl, quinolyl, isoquinolyl, isoxazolyl, pyrazolyl, imidazolyl, thienyl, thiadiazolyl, furyl or pyrrolyl, each of which is optionally substituted.

12. (canceled)

13. A method of treating a disorder responsive to the induction of apoptosis in an animal suffering therefrom, comprising administering to an animal in need of such treatment an effective amount of a compound selected from the group consisting of:
3-(4-Methylphenyl)-6-(3,4-dimethoxyphenyl)-7H-[1,2,4]triazolo[3,4-b][1,3,4]thiadiazine;
3-(4-Methylphenyl)-6-(4-ethoxyphenyl)-7H-[1,2,4]triazolo[3,4-b][1,3,4]thiadiazine;
3-(4-Methylphenyl)-6-(3,4-methylenedioxyphenyl)-7H-[1,2,4]triazolo[3,4-b][1,3,4]thiadiazine;
3-(4-Chlorophenyl)-6-(4-methoxyphenyl)-7H-[1,2,4]triazolo[3,4-b][1,3,4]thiadiazine;
3-(4-Chlorophenyl)-6-(3,4-dimethoxyphenyl)-7H-[1,2,4]triazolo[3,4-b][1,3,4]thiadiazine;
3-(4-Chlorophenyl)-6-(4-ethoxyphenyl)-7H-[1,2,4]triazolo[3,4-b][1,3,4]thiadiazine;
3-(4-Chlorophenyl)-6-(3,4-methylenedioxyphenyl)-7H-[1,2,4]triazolo[3,4-b][1,3,4]thiadiazine;
3-(4-Methoxyphenyl)-6-(2,5-dimethoxyphenyl)-7H-[1,2,4]triazolo[3,4-b][1,3,4]thiadiazine;
3-(4-Methoxyphenyl)-6-(4-ethoxyphenyl)-7H-[1,2,4]triazolo[3,4-b][1,3,4]thiadiazine;
3-(4-Methoxyphenyl)-6-(3,4-methylenedioxyphenyl)-7H-[1,2,4]triazolo[3,4-b][1,3,4]thiadiazine;
3-(2-Methylphenyl)-6-(3,4-dimethoxyphenyl)-7H-[1,2,4]triazolo[3,4-b][1,3,4]thiadiazine;
3-(2-Methylphenyl)-6-(4-ethoxyphenyl)-7H-[1,2,4]triazolo[3,4-b][1,3,4]thiadiazine;
3-(2-Methylphenyl)-6-(3,4-methylenedioxyphenyl)-7H-[1,2,4]triazolo[3,4-b][1,3,4]thiadiazine;
3-(2-Chlorophenyl)-6-(3,4-dimethoxyphenyl)-7H-[1,2,4]triazolo[3,4-b][1,3,4]thiadiazine;
3-(2-Chlorophenyl)-6-(4-ethoxyphenyl)-7H-[1,2,4]triazolo[3,4-b][1,3,4]thiadiazine;
3-(2-Chlorophenyl)-6-(3,4-methylenedioxyphenyl)-7H-[1,2,4]triazolo[3,4-b][1,3,4]thiadiazine;
3-(2-Chlorophenyl)-6-(4-bromophenyl)-7H-[1,2,4]triazolo[3,4-b][1,3,4]thiadiazine;
3-(3-Chlorophenyl)-6-(4-methoxyphenyl)-7H-[1,2,4]triazolo[3,4-h][1,3,4]thiadiazine;
3-(3-Chlorophenyl)-6-(3,4-methylenedioxyphenyl)-7H-[1,2,4]triazolo[3,4-b][1,3,4]thiadiazine;
3-(3-Methylphenyl)-6-(3,4-methylenedioxyphenyl)-7H-[1,2,4]triazolo[3,4-b][1,3,4]thiadiazine;
3-(3-Methylphenyl)-6-(2-naphthyl)-7H-[1,2,4]triazolo[3,4-b][1,3,4]thiadiazine;
3-(2-Methoxyphenyl)-6-(3,4-methylenedioxyphenyl)-7H-[1,2,4]triazolo[3,4-b][1,3,4]thiadiazine;
3-(2-Methoxyphenyl)-6-(4-bromophenyl)-7H-[1,2,4]triazolo[3,4-b][1,3,4]thiadiazine;
3-(3-Methoxyphenyl)-6-(4-methoxyphenyl)-7H-[1,2,4]triazolo[3,4-b][1,3,4]thiadiazine;
3-(3-Methoxyphenyl)-6-(3,4-dimethoxyphenyl)-7H-[1,2,4]triazolo[3,4-b][1,3,4]thiadiazine;
3-(3-Methoxyphenyl)-6-(4-ethoxyphenyl)-7H-[1,2,4]triazolo[3,4-b][1,3,4]thiadiazine;
3-(3-Methoxyphenyl)-6-(3,4-methylenedioxyphenyl)-7H-[1,2,4]triazolo[3,4-b][1,3,4]thiadiazine;
3-(3-Methoxyphenyl)-6-(2-naphthyl)-7H-[1,2,4]triazolo[3,4-b][1,3,4]thiadiazine;
3-(3-Ethoxyphenyl)-6-(3,4-dihydroxyphenyl)-7H-[1,2,4]triazolo[3,4-b][1,3,4]thiadiazine;
3-(3-Ethoxyphenyl)-6-(4-methoxyphenyl)-7H-[1,2,4]triazolo[3,4-b][1,3,4]thiadiazine;
3-(3-Ethoxyphenyl)-6-(3,4-methylenedioxyphenyl)-7H-[1,2,4]triazolo[3,4-b][1,3,4]thiadiazine;
3-(4-Ethoxyphenyl)-6-(3,4-dihydroxyphenyl)-7H-[1,2,4]triazolo[3,4-b][1,3,4]thiadiazine;
3-(2,4-Dichlorophenyl)-6-(3,4-dimethoxyphenyl)-7H-[1,2,4]triazolo[3,4-b][1,3,4]thiadiazine;
3-(2,4-Dichlorophenyl)-6-(4-ethoxyphenyl)-7H-[1,2,4]triazolo[3,4-b][1,3,4]thiadiazine;
3-(2,4-Dichlorophenyl)-6-(3,4-methylenedioxyphenyl)-7H-[1,2,4]triazolo[3,4-b][1,3,4]thiadiazine;
3-(2,4-Dichlorophenyl)-6-phenyl-7H-[1,2,4]triazolo[3,4-b][1,3,4]thiadiazine;
3-(2-Methoxyphenyl)-6-(3,4-dimethoxyphenyl)-7H-[1,2,4]triazolo[3,4-b][1,3,4]thiadiazine;
3-(3,4,5-Trimethoxyphenyl)-6-(3,4-dimethoxyphenyl)-7H-[1,2,4]triazolo[3,4-b][1,3,4]thiadiazine;
3-(3,4,5-Trimethoxyphenyl)-6-(3,4-methylenedioxyphenyl)-7H-[1,2,4]triazolo[3,4-b][1,3,4]thiadiazine;
3-(3,4-Dimethoxyphenyl)-6-(3,4-dimethoxyphenyl)-7H-[1,2,4]triazolo[3,4-b][1,3,4]thiadiazine;
3-(3,4-Dimethoxyphenyl)-6-(3,4-methylenedioxyphenyl)-7H-[1,2,4]triazolo[3,4-b][1,3,4]thiadiazine;
3-Phenyl-6-(3,4-methylenedioxyphenyl)-7H-[1,2,4]triazolo[3,4-b][1,3,4]thiadiazine;
3-(2-Fluorophenyl)-6-(3,4-methylenedioxyphenyl)-7H-[1,2,4]triazolo[3,4-b][1,3,4]thiadiazine;
3-(3-Isopropyloxyphenyl)-6-(3,4-methylenedioxyphenyl)-7H-[1,2,4]triazolo[3,4-b][1,3,4]thiadiazine;
3-(3,4,5-Trimethoxyphenyl)-6-phenyl-7H-[1,2,4]triazolo[3,4-b][1,3,4]thiadiazine;
3-(3,4,5-Trimethoxyphenyl)-6-(4-ethoxyphenyl)-7H-[1,2,4]triazolo[3,4-b][1,3,4]thiadiazine;
3-(3,4,5-Trimethoxyphenyl)-6-(4-bromophenyl)-7H-[1,2,4]triazolo[3,4-b][1,3,4]thiadiazine;
3-(3,4,5-Trimethoxyphenyl)-6-(4-fluorophenyl)-7H-[1,2,4]triazolo[3,4-b][1,3,4]thiadiazine;
3-(3,4,5-Trimethoxyphenyl)-6-(4-chlorophenyl)-7H-[1,2,4]triazolo[3,4-b][1,3,4]thiadiazine;
3-(3,4,5-Trimethoxyphenyl)-6-(3-bromophenyl)-7H-[1,2,4]triazolo[3,4-b][1,3,4]thiadiazine;
3-(3,4,5-Trimethoxyphenyl)-6-(2-naphthyl)-7H-[1,2,4]triazolo[3,4-b][1,3,4]thiadiazine;
3-(3,4,5-Trimethoxyphenyl)-6-(3,4-dihydroxyphenyl)-7H-[1,2,4]triazolo[3,4-b][1,3,4]thiadiazine;
3-(3,4,5-Trimethoxyphenyl)-6-(4-methoxyphenyl)-7H-[1,2,4]triazolo[3,4-b][1,3,4]thiadiazine;
3-(2-Methoxyphenyl)-6-(4-ethoxyphenyl)-7H-[1,2,4]triazolo[3,4-b][1,3,4]thiadiazine;
3-(2-Chlorophenyl)-6-(4-methoxyphenyl)-7H-[1,2,4]triazolo[3,4-b][1,3,4]thiadiazine;
3-(3-Methoxyphenyl)-6-(3,4-dihydroxyphenyl)-7H-[1,2,4]triazolo[3,4-b][1,3,4]thiadiazine;
3-(3-Chlorophenyl)-6-(4-ethoxyphenyl)-7H-[1,2,4]triazolo[3,4-b][1,3,4]thiadiazine;
3-(3-Methoxyphenyl)-6-(4-bromophenyl)-7H-[1,2,4]triazolo[3,4-b][1,3,4]thiadiazine;
3-(3-Ethoxyphenyl)-6-(4-ethoxyphenyl)-7H-[1,2,4]triazolo[3,4-b][1,3,4]thiadiazine;
3-(4-Ethoxyphenyl)-6-(3,4-methylenedioxyphenyl)-7H-[1,2,4]triazolo[3,4-b][1,3,4]thiadiazine;
3-(3-Bromophenyl)-6-(4-methoxyphenyl)-7H-[1,2,4]triazolo[3,4-b][1,3,4]thiadiazine;
3-Ethyl-6-(4-methoxyphenyl)-7H-[1,2,4]triazolo[3,4-b][1,3,4]thiadiazine;
3-(3-Isopropyloxyphenyl)-6-(4-methoxyphenyl)-7H-[1,2,4]triazolo[3,4-b][1,3,4]thiadiazine;
3-(2-Fluorophenyl)-6-(4-methoxyphenyl)-7H-[1,2,4]triazolo[3,4-b][1,3,4]thiadiazine;
3-Cyclohexyl-6-(3,4-methylenedioxyphenyl)-7H-[1,2,4]triazolo[3,4-b][1,3,4]thiadiazine;
3-(2-Methylphenyl)-6-(4-methoxyphenyl)-7H-[1,2,4]triazolo[3,4-b][1,3,4]thiadiazine;
3-(3-Methylphenyl)-6-(4-ethoxyphenyl)-7H-[1,2,4]triazolo[3,4-b][1,3,4]thiadiazine;
3-(4-Fluorophenyl)-6-(4-ethoxyphenyl)-7H-[1,2,4]triazolo[3,4-b][1,3,4]thiadiazine;
3-(2-Methylphenyl)-6-(4-methoxyphenyl)-7H-[1,2,4]triazolo[3,4-b][1,3,4]thiadiazine;
3-(3-Methylphenyl)-6-(4-methoxyphenyl)-7H-[1,2,4]triazolo[3,4-b][1,3,4]thiadiazine;
3-Cyclohexyl-6-(4-methoxyphenyl)-7H-[1,2,4]triazolo[3,4-b][1,3,4]thiadiazine;
3-(4-Fluorophenyl)-6-(4-methoxyphenyl)-7H-[1,2,4]triazolo[3,4-b][1,3,4]thiadiazine;
3-(Furan-2-yl)-6-(4-methoxyphenyl)-7H-[1,2,4]triazolo[3,4-b][1,3,4]thiadiazine;
3-(4-Methylphenyl)-6-(4-methoxyphenyl)-7H-[1,2,4]triazolo[3,4-b][1,3,4]thiadiazine;
3-(1,4,5,6-Tetrahydrocyclopenta[c]pyrazol-3-yl)-6-(4-methoxyphenyl)-7H-[1,2,4]triazolo[3,4-b][1,3,4]thiadiazine;
3-(1,4,5,6-Tetrahydrocyclopenta[c]pyrazol-3-yl)-6-(3,4-methylenedioxyphenyl)-7H-[1,2,4]triazolo[3,4-b][1,3,4]thiadiazine;
3-(2-Methoxyphenyl)-6-(4-nitrophenyl)-7H-[1,2,4]triazolo[3,4-b][1,3,4]thiadiazine;
3-(4-Methylphenyl)-6-(4-difluoromethoxyphenyl)-7H-[1,2,4]triazolo[3,4-b][1,3,4]thiadiazine;
3-(4-Bromophenyl)-6-(4-ethoxyphenyl)-7H-[1,2,4]triazolo[3,4-b][1,3,4]thiadiazine;
3-(2-Bromophenyl)-6-(4-ethoxyphenyl)-7H-[1,2,4]triazolo[3,4-b][1,3,4]thiadiazine;
3-(4-Methoxyphenyl)-6-(4-methoxyphenyl)-7H-[1,2,4]triazolo[3,4-b][1,3,4]thiadiazine;
3-(3-Fluorophenyl)-6-(4-methoxyphenyl)-7H-[1,2,4]triazolo[3,4-b][1,3,4]thiadiazine;
3-(2-Bromophenyl)-6-(4-methoxyphenyl)-7H-[1,2,4]triazolo[3,4-b][1,3,4]thiadiazine;
3-(4-Bromophenyl)-6-(4-methoxyphenyl)-7H-[1,2,4]triazolo[3,4-h][1,3,4]thiadiazine;
3-(4-Methoxyphenyl)-6-(4-methylphenyl)-7H-[1,2,4]triazolo[3,4-b][1,3,4]thiadiazine;
3-(3-Fluorophenyl)-6-(4-ethylphenyl)-7H-[1,2,4]triazolo[3,4-b][1,3,4]thiadiazine;
6-(5-Bromothiophen-2-yl)-3-(3-methoxyphenyl)-7H-[1,2,4]triazolo[3,4-b][1,3,4]thiadiazine;
6-(4-Methylphenyl)-3-phenyl-7H-[1,2,4]triazolo[3,4-b][1,3,4]thiadiazine;
6-(4-Chlorophenyl)-3-(2-methoxyphenyl)-7H-[1,2,4]triazolo[3,4-b][1,3,4]thiadiazine;
3-(2-Methoxyphenyl)-6-phenyl-7H-[1,2,4]triazolo[3,4-b][1,3,4]thiadiazine;
3-(2,4-Dichlorophenyl)-6-(4-methylphenyl)-7H-[1,2,4]triazolo[3,4-b][1,3,4]thiadiazine;
3-(2,3-Dichlorophenyl)-6-(4-methoxyphenyl)-7H-[1,2,4]triazolo[3,4-b][1,3,4]thiadiazine;
3-(2,4-Dichlorophenyl)-6-(4-methoxyphenyl)-7H-[1,2,4]triazolo[3,4-b][1,3,4]thiadiazine;
3-(2,4-Dichlorophenyl)-6-(3-methoxyphenyl)-7H-[1,2,4]triazolo[3,4-b][1,3,4]thiadiazine;
6-(4-Fluorophenyl)-3-(2-methoxyphenyl)-7H-[1,2,4]triazolo[3,4-b][1,3,4]thiadiazine;
6-(3,4-Dichlorophenyl)-3-(2-methoxyphenyl)-7H-[1,2,4]triazolo[3,4-b][1,3,4]thiadiazine;
3-(3,5-dimethoxyphenyl)-6-(3,4-methylenedioxyphenyl)-7H-[1,2,4]triazolo[3,4-b][1,3,4]thiadiazine;
3-(3-Chloro-4,5-dimethoxyphenyl)-6-(3,4-methylenedioxyphenyl)-7H-[1,2,4]triazolo[3,4-h][1,3,4]thiadiazine;
3-(3-Chloro-4,5-dimethoxyphenyl)-6-(4-methoxyphenyl)-7H-[1,2,4]triazolo[3,4-b][1,3,4]thiadiazine;
3-(4-Chlorophenyl)-6-(4-hydroxyphenyl)-7H-[1,2,4]triazolo[3,4-b][1,3,4]thiadiazine;
6-(4-Hydroxyphenyl)-3-(2-methoxyphenyl)-7H-[1,2,4]triazolo[3,4-h][1,3,4]thiadiazine;
3-(2-Methoxyphenyl)-6-(4-methylphenyl)-7H-[1,2,4]triazolo[3,4-b][1,3,4]thiadiazine;
3-(2-Methoxyphenyl)-6-(4-(2-dimethylaminoethoxy)phenyl)-7H-[1,2,4]triazolo[3,4-h][1,3,4]thiadiazine;
6-(4-(2-Hydroxyethoxy)phenyl)-3-(2-methoxyphenyl)-7H-[1,2,4]triazolo[3,4-b][1,3,4]thiadiazine;
3-(2-Chlorophenyl)-6-(4-methylphenyl)-7H-[1,2,4]triazolo[3,4-h][1,3,4]thiadiazine;
3-(3,5-Dimethoxyphenyl)-6-(4-methylphenyl)-7H-[1,2,4]triazolo[3,4-b][1,3,4]thiadiazine;
3-(3,5-Dimethoxy-4-hydroxyphenyl)-6-(4-methylphenyl)-7H-[1,2,4]triazolo[3,4-b][1,3,4]thiadiazine;
3-(3,4-Dimethoxyphenyl)-6-(4-methylphenyl)-7H-[1,2,4]triazolo[3,4-b][1,3,4]thiadiazine;
3-(2,4-Dimethoxyphenyl)-6-(4-methylphenyl)-7H-[1,2,4]triazolo[3,4-b][1,3,4]thiadiazine;
3-(3,5-Dimethoxy-4-(2-dimethylaminoethoxy)phenyl)-6-(4-methylphenyl)-7H-[1,2,4]triazolo[3,4-b][1,3,4]thiadiazine;
3-(3,4,5-Trimethoxyphenyl)-6-(4-methylphenyl)-7H-[1,2,4]triazolo[3,4-b][1,3,4]thiadiazine;
3-(2,4-Dimethoxyphenyl)-6-(4-methoxyphenyl)-7H-[1,2,4]triazolo[3,4-b][1,3,4]thiadiazine;
3-(2-Ethoxyphenyl)-6-(4-methylphenyl)-7H-[1,2,4]triazolo[3,4-b][1,3,4]thiadiazine;
3-(3,5-Dimethoxyphenyl)-6-(4-methoxyphenyl)-7H-[1,2,4]triazolo[3,4-b][1,3,4]thiadiazine;
6-(4-Diethylaminophenyl)-3-(3,4,5-trimethoxyphenyl)-7H-[1,2,4]triazolo[3,4-h][1,3,4]thiadiazine;
6-(4-Diethylaminophenyl)-3-(3,4-dimethoxyphenyl)-7H-[1,2,4]triazolo[3,4-b][1,3,4]thiadiazine;
6-(4-Diethylaminophenyl)-3-(2-methoxyphenyl)-7H-[1,2,4]triazolo[3,4-b][1,3,4]thiadiazine;
3-(2-Methoxyphenyl)-6-(4-(pyrrolidin-1-yl)phenyl)-7H-[1,2,4]triazolo[3,4-b][1,3,4]thiadiazine;
3-(2-Methoxyphenyl)-6-(4-morpholinophenyl)-7H-[1,2,4]triazolo[3,4-b][1,3,4]thiadiazine;
6-(4-Diethylaminophenyl)-3-(3,5-dimethoxyphenyl)-7H-[1,2,4]triazolo[3,4-b][1,3,4]thiadiazine;
6-(4-Diethylaminophenyl)-3-(2,4-dimethoxyphenyl)-7H-[1,2,4]triazolo[3,4-b][1,3,4]thiadiazine;
3-(3,4-Dimethoxyphenyl)-6-(4-nitrophenyl)-7H-[1,2,4]triazolo[3,4-b][1,3,4]thiadiazine;
6-(4-Aminophenyl)-3-(3,4-dimethoxyphenyl)-7H-[1,2,4]triazolo[3,4-b][1,3,4]thiadiazine;
6-(4-Dimethylminophenyl)-3-(3,4-dimethoxyphenyl)-7H-[1,2,4]triazolo[3,4-b][1,3,4]thiadiazine;
6-(4-Azidophenyl)-3-(3,4-dimethoxyphenyl)-7H-[1,2,4]triazolo[3,4-b][1,3,4]thiadiazine;
6-(4-Acetamidophenyl)-3-(3,4-dimethoxyphenyl)-7H-[1,2,4]triazolo[3,4-b][1,3,4]thiadiazine;
6-(2,3-Dihydro-2-oxobenzo[d]oxazol-6-yl)-3-(2,4-Dimethoxyphenyl)-7H-[1,2,4]triazolo[3,4-b][1,3,4]thiadiazine;
6-(2,3-Dihydro-2-oxobenzo[d]oxazol-6-yl)-3-(3,5-dimethoxyphenyl)-7H-[1,2,4]triazolo[3,4-b][1,3,4]thiadiazine;
6-(2,3-Dihydro-2-oxobenzo[d]oxazol-6-yl)-3-(3,4-dimethoxyphenyl)-7H-[1,2,4]triazolo[3,4-b][1,3,4]thiadiazines;
3-(2-Methoxyphenyl)-6-(4-methoxyphenyl)-[1,2,4]triazolo[4,3-b][1,2,4]triazin-7(8H)-one;
3-(3,4,5-Trimethoxyphenyl)-6-(4-methylphenyl)-1H-[1,2,4]triazolo[4,3-b][1,2,4]triazin-7-one;
3-(2-Methoxyphenyl)-6-(4-methylphenyl)-1H-[1,2,4]triazolo[4,3-b][1,2,4]triazin-7-one;
3-(2-Methoxyphenyl)-6-(2,4-dimethylphenyl)-7H-[1,2,4]triazolo[3,4-b][1,3,4]thiadiazine;
3-(2-Methoxyphenyl)-6-(3,4-dimethylphenyl)-7H-[1,2,4]triazolo[3,4-b][1,3,4]thiadiazine;
6-(2-Methoxy-4-methylphenyl)-3-(2-methoxyphenyl)-7H-[1,2,4]triazolo[3,4-b][1,3,4]thiadiazine;
6-(2,3,4-Trimethoxyphenyl)-3-(2-methoxyphenyl)-7H-[1,2,4]triazolo[3,4-b][1,3,4]thiadiazine;
6-(2,3-Dihydrobenzo[b][1,4]dioxin-6-yl)-3-(2-methoxyphenyl)-7H-[1,2,4]triazolo[3,4-b][1,3,4]thiadiazine;
6-(4-Isopropylphenyl)-3-(2-methoxyphenyl)-7H-[1,2,4]triazolo[3,4-b][1,3,4]thiadiazine;
6-(3-Methoxy-4-nitrophenyl)-3-(2-methoxyphenyl)-7H-[1,2,4]triazolo[3,4-b][1,3,4]thiadiazine;
3-(2-Methoxyphenyl)-6-(4-methoxyphenyl)-7H-[1,2,4]triazolo[3,4-b]-[1,3,4]thiadiazine;
6-(2,3-Dihydro-2-oxobenzo[d]oxazol-6-yl)-3-(2-methoxyphenyl)-7H-[1,2,4]triazolo[3,4-b][1,3,4]thiadiazine;
6-(4-(Trifluoromethyl)phenyl)-3-(2-methoxyphenyl)-7H-[1,2,4]triazolo[3,4-b][1,3,4]thiadiazine;
6-(5-Chloro-2-methoxy-4-methylphenyl)-3-(2-methoxyphenyl)-7H-[1,2,4]triazolo[3,4-b][1,3,4]thiadiazine;
3-(2-Methoxyphenyl)-6-(4-(trifluoromethoxy)phenyl)-7H-[1,2,4]triazolo[3,4-b][1,3,4]thiadiazine;
6-(4-tert-Butylphenyl)-3-(2-methylphenyl)-7H-[1,2,4]triazolo[3,4-b][1,3,4]thiadiazine;
6-(4-(N,N-Diethylaminosulfonyl)phenyl)-3-(2-methylphenyl)-7H-[1,2,4]triazolo[3,4-b][1,3,4]thiadiazine;
6-(4-Methyl-3-nitrophenyl)-3-(2-methylphenyl)-7H-[1,2,4]triazolo[3,4-b][1,3,4]thiadiazine;
3-(2-Methoxyphenyl)-6-(4-methyl-3-nitrophenyl)-7H-[1,2,4]triazolo[3,4-b][1,3,4]thiadiazine;
6-(4-Chloro-3-nitrophenyl)-3-(2-methoxyphenyl)-7H-[1,2,4]triazolo[3,4-b][1,3,4]thiadiazine;
6-(4-Acetamido-3-nitrophenyl)-3-(2-methoxyphenyl)-7H-[1,2,4]triazolo[3,4-b][1,3,4]thiadiazine;
6-(4-Amino-3-methoxyphenyl)-3-(2-methoxyphenyl)-7H-[1,2,4]triazolo[3,4-b][1,3,4]thiadiazine;
6-(3-Amino-4-methylphenyl)-3-(2-methoxyphenyl)-7H-[1,2,4]triazolo[3,4-b][1,3,4]thiadiazin;
6-(3-Amino-4-chlorophenyl)-3-(2-methoxyphenyl)-7H-[1,2,4]triazolo[3,4-b][1,3,4]thiadiazine;
6-(3,4-Diaminophenyl)-3-(2-methoxyphenyl)-7H-[1,2,4]triazolo[3,4-b][1,3,4]thiadiazine;
6-(3-Amino-4-methylphenyl)-3-(2-methylphenyl)-7H-[1,2,4]triazolo[3,4-b][1,3,4]thiadiazine;
3-(5-Chloro-2-methoxyphenyl)-6-(4-methylphenyl)-7H-[1,2,4]triazolo[3,4-b][1,3,4]thiadiazine;
3-(2-Methoxyphenyl)-6-(3-methoxylphenyl)-7H-[1,2,4]triazolo[3,4-b][1,3,4]thiadiazine;
3-(2-Methoxyphenyl)-6-(2-methoxylphenyl)-7H-[1,2,4]triazolo[3,4-b][1,3,4]thiadiazine;
3-(2,3-Dimethoxyphenyl)-6-(4-methylphenyl)-7H-[1,2,4]triazolo[3,4-b][1,3,4]thiadiazine;
6-(4-Aminophenyl)-3-(3,4,5-trimethoxyphenyl)-7H-[1,2,4]triazolo[3,4-b][1,3,4]thiadiazine;
6-(4-Dimethylaminophenyl)-3-(3,4,5-trimethoxyphenyl)-7H-[1,2,4]triazolo[3,4-b][1,3,4]thiadiazine;
3-(2-Fluorophenyl)-6-(4-methylphenyl)-7H-[1,2,4]triazolo[3,4-b][1,3,4]thiadiazine;
3-(2,5-Dimethoxyphenyl)-6-(4-methylphenyl)-7H-[1,2,4]triazolo[3,4-b][1,3,4]thiadiazine;
3-(2-Methylphenyl)-6-(4-methylphenyl)-7H-[1,2,4]triazolo[3,4-b][1,3,4]thiadiazine;
3-(2-Methylthiophenyl)-6-(4-methylphenyl)-7H-[1,2,4]triazolo[3,4-b][1,3,4]thiadiazine;
3-(2-Methylsulfinylphenyl)-6-(4-methylphenyl)-7H-[1,2,4]triazolo[3,4-b][1,3,4]thiadiazine;
3-(2-Methylsulfonylphenyl)-6-(4-methylphenyl)-7H-[1,2,4]triazolo[3,4-b][1,3,4]thiadiazine;
3-(2-Aminophenyl)-6-(4-methylphenyl)-7H-[1,2,4]triazolo[3,4-b][1,3,4]thiadiazine;
6-(4-Aminophenyl)-3-(2-methoxyphenyl)-7H-[1,2,4]triazolo[3,4-b][1,3,4]thiadiazine;
6-(4-Azidophenyl)-3-(2-methoxyphenyl)-7H-[1,2,4]triazolo[3,4-b][1,3,4]thiadiazine;
6-(4-dimethylaminophenyl)-3-(2-methoxyphenyl)-7H-[1,2,4]triazolo[3,4-b][1,3,4]thiadiazine;
6-(4-Amino-3,5-dibromophenyl)-3-(2-methoxyphenyl)-7H-[1,2,4]triazolo[3,4-b][1,3,4]thiadiazine;
6-(4-Amino-3,5-diiodophenyl)-3-(2-methoxyphenyl)-7H-[1,2,4]triazolo[3,4-b][1,3,4]thiadiazine;
6-(4-Isopropylaminophenyl)-3-(2-methoxyphenyl)-7H-[1,2,4]triazolo[3,4-b][1,3,4]thiadiazine;
3-(4-Chloro-2-methylphenyl)-6-(4-methylphenyl)-7H-[1,2,4]triazolo[3,4-b][1,3,4]thiadiazine;
3-(2-Methoxyphenyl)-6-(3-methylphenyl)-7H-[1,2,4]triazolo[3,4-b][1,3,4]thiadiazine;
3-(2-Methylfuran-3-yl)-6-(4-methylphenyl)-7H-[1,2,4]triazolo[3,4-b][1,3,4]thiadiazine;
3-(2-Methylaminophenyl)-6-(4-methylphenyl)-7H-[1,2,4]triazolo[3,4-b][1,3,4]thiadiazine;
6-(4-Methoxycarbonylphenyl)-3-(2-methoxyphenyl)-7H-[1,2,4]triazolo[3,4-b][1,3,4]thiadiazine;
3-(2-Methoxyphenyl)-6-(1-methyl-1H-pyrrol-3-yl)-7H-[1,2,4]triazolo[3,4-b][1,3,4]thiadiazine;
6-(4-Cyanophenyl)-3-(2-methoxyphenyl)-7H-[1,2,4]triazolo[3,4-b][1,3,4]thiadiazine;
5,6-Dihydro-3-(2-methoxyphenyl)-6-(4-methylphenyl)-7H-[1,2,4]triazolo[3,4-b][1,3,4]thiadiazine;
6-(4-Carboxylphenyl)-3-(2-methoxyphenyl)-7H-[1,2,4]triazolo[3,4-b][1,3,4]thiadiazine;
3-(4-Chloro-2-methylphenyl)-6-(4-methyl-3-nitrophenyl)-7H-[1,2,4]triazolo[3,4-b][1,3,4]thiadiazine;
6-(3-Amino-4-methylphenyl)-3-(4-chloro-2-methylphenyl)-7H-[1,2,4]triazolo[3,4-b][1,3,4]thiadiazine;
6-(4-Methylphenyl)-3-(1H-pyrrol-2-yl)-7H-[1,2,4]triazolo[3,4-b][1,3,4]thiadiazine;
3-(4-Methyl-1,2,3-thiadiazol-5-yl)-6-(4-methylphenyl)-7H-[1,2,4]triazolo[3,4-b][1,3,4]thiadiazine;
3-(1-Methyl-1H-pyrrol-2-yl)-6-(4-methylphenyl)-7H-[1,2,4]triazolo[3,4-h][1,3,4]thiadiazine;
3-(1-Methyl-4-nitro-1H-pyrrol-2-yl)-6-(4-methylphenyl)-7H-[1,2,4]triazolo[3,4-b][1,3,4]thiadiazine;
6-(7-Bromoindolin-5-yl)-3-(2-methoxyphenyl)-7H-[1,2,4]triazolo[3,4-b][1,3,4]thiadiazine;
3-(4-Chloro-2-methoxyphenyl)-6-(4-methylphenyl)-7H-[1,2,4]triazolo[3,4-b][1,3,4]thiadiazine;
3-(1-Methyl-4-amino-1H-pyrrol-2-yl)-6-(4-methylphenyl)-7H-[1,2,4]triazolo[3,4-b][1,3,4]thiadiazine;
3-(2-Methyl-imidazo[1,2-a]pyridin-3-yl)-6-(4-methylphenyl)-7H-[1,2,4]triazolo[3,4-b][1,3,4]thiadiazine;
or a pharmaceutically acceptable salt or prodrug thereof.

14. The method of claim 9 or 13, wherein said disorder is cancer.

15-19. (canceled)

20. The method according to claim 14, wherein said compound is administered together with at least one compound selected from the group consisting of busulfan, cis-platin, mitomycin C, carboplatin, colchicine, vinblastine, paclitaxel, docetaxel, camptothecin, topotecan, doxorubicin, etoposide, 5-azacytidine, 5-fluorouracil, methotrexate, 5-fluoro-2′-deoxy-uridine, ara-C, hydroxyurea, thioguanine, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen, Herceptin®, Rituxan®, arsenic trioxide, gemcitabine, doxazosin, terazosin, tamsulosin, CB-64D, CB-184, haloperidol, lovastatin, simvastatin, pravastatin, fluvastatin, atorvastatin, cerivastatin, amprenavir, abacavir, CGP-73547, CGP-61755, DMP-450, indinavir, nelfinavir, tipranavir, ritonavir, saquinavir, ABT-378, AG 1776, BMS-232,632, bexarotene, tretinoin, 13-cis-retinoic acid, 9-cis-retinoic acid, α-difluoromethylornithine, ILX23-7553, fenretinide, N-4-carboxyphenyl retinamide, lactacystin, MG-132, PS-341, Gleevec®, ZD1839 (Iressa), SH268, genistein, CEP2563, SU6668, SU11248, EMD121974, R115777, SCH66336, L-778,123, BAL9611, TAN-1813, flavopiridol, UCN-01, roscovitine, olomoucine, celecoxib, valecoxib, rofecoxib and alanosine.

21. The method of claim 14, further comprising treating said animal with radiation-therapy.

22. The method of claim 14, wherein said compound is administered after surgical treatment of said animal for said cancer.

23. The method of claim 9 or 13, wherein said disorder is an autoimmune disease.

24. (canceled)

25. The method of claim 9 or 13, wherein said disorder is rheumatoid arthritis.

26. The method of claim 9 or 13, wherein said disorder is an inflammatory disease.

27. The method of claim 9 or 13, wherein said disorder is a skin disease.

28. (canceled)

29. A compound selected from the group consisting of:
3-(3,5-dimethoxyphenyl)-6-(3,4-methylenedioxyphenyl)-7H-[1,2,4]triazolo[3,4-b][1,3,4]thiadiazine;
3-(3-Chloro-4,5-dimethoxyphenyl)-6-(3,4-methylenedioxyphenyl)-7H-[1,2,4]triazolo[3,4-b][1,3,4]thiadiazine;
3-(3-Chloro-4,5-dimethoxyphenyl)-6-(4-methoxyphenyl)-7H-[1,2,4]triazolo[3,4-b][1,3,4]thiadiazine;
3-(4-Chlorophenyl)-6-(4-hydroxyphenyl)-7H-[1,2,4]triazolo[3,4-b][1,3,4]thiadiazine;
6-(4-Hydroxyphenyl)-3-(2-methoxyphenyl)-7H-[1,2,4]triazolo[3,4-b][1,3,4]thiadiazine;
3-(2-Methoxyphenyl)-6-(4-methylphenyl)-7H-[1,2,4]triazolo[3,4-b][1,3,4]thiadiazine;
3-(2-Methoxyphenyl)-6-(4-(2-dimethylaminoethoxy)phenyl)-7H-[1,2,4]triazolo[3,4-b][1,3,4]thiadiazine;
6-(4-(2-Hydroxyethoxy)phenyl)-3-(2-methoxyphenyl)-7H-[1,2,4]triazolo[3,4-b][1,3,4]thiadiazine;
3-(2-Chlorophenyl)-6-(4-methylphenyl)-7H-[1,2,4]triazolo[3,4-b][1,3,4]thiadiazine;
3-(3,5-Dimethoxyphenyl)-6-(4-methylphenyl)-7H-[1,2,4]triazolo[3,4-b][1,3,4]thiadiazine;
3-(3,5-Dimethoxy-4-hydroxyphenyl)-6-(4-methylphenyl)-7H-[1,2,4]triazolo[3,4-b][1,3,4]thiadiazine;
3-(3,4-Dimethoxyphenyl)-6-(4-methylphenyl)-7H-[1,2,4]triazolo[3,4-b][1,3,4]thiadiazine;
3-(2,4-Dimethoxyphenyl)-6-(4-methylphenyl)-7H-[1,2,4]triazolo[3,4-b][1,3,4]thiadiazine;
3-(3,5-Dimethoxy-4-(2-dimethylaminoethoxy)phenyl)-6-(4-methylphenyl)-7H-[1,2,4]triazolo[3,4-b][1,3,4]thiadiazine;
3-(3,4,5-Trimethoxyphenyl)-6-(4-methylphenyl)-7H-[1,2,4]triazolo[3,4-b][1,3,4]thiadiazine;
3-(2,4-Dimethoxyphenyl)-6-(4-methoxyphenyl)-7H-[1,2,4]triazolo[3,4-b][1,3,4]thiadiazine;
3-(2-Ethoxyphenyl)-6-(4-methylphenyl)-7H-[1,2,4]triazolo[3,4-b][1,3,4]thiadiazine;
3-(3,5-Dimethoxyphenyl)-6-(4-methoxyphenyl)-7H-[1,2,4]triazolo[3,4-b][1,3,4]thiadiazine;
6-(4-Diethylaminophenyl)-3-(3,4,5-trimethoxyphenyl)-7H-[1,2,4]triazolo[3,4-b][1,3,4]thiadiazine;
6-(4-Diethylaminophenyl)-3-(3,4-dimethoxyphenyl)-7H-[1,2,4]triazolo[3,4-b][1,3,4]thiadiazine;
6-(4-Diethylaminophenyl)-3-(2-methoxyphenyl)-7H-[1,2,4]triazolo[3,4-b][1,3,4]thiadiazine;
3-(2-Methoxyphenyl)-6-(4-(pyrrolidin-1-yl)phenyl)-7H-[1,2,4]triazolo[3,4-b][1,3,4]thiadiazine;
3-(2-Methoxyphenyl)-6-(4-morpholinophenyl)-7H-[1,2,4]triazolo[3,4-b][1,3,4]thiadiazine;
6-(4-Diethylaminophenyl)-3-(3,5-dimethoxyphenyl)-7H-[1,2,4]triazolo[3,4-b][1,3,4]thiadiazine;
6-(4-Diethylaminophenyl)-3-(2,4-dimethoxyphenyl)-7′-[1,2,4]triazolo[3,4-b][1,3,4]thiadiazine;
3-(3,4-Dimethoxyphenyl)-6-(4-nitrophenyl)-7H-[1,2,4]triazolo[3,4-b][1,3,4]thiadiazine;
6-(4-Aminophenyl)-3-(3,4-dimethoxyphenyl)-7H-[1,2,4]triazolo[3,4-b][1,3,4]thiadiazine;
6-(4-Dimethylminophenyl)-3-(3,4-dimethoxyphenyl)-7H-[1,2,4]triazolo[3,4-b][1,3,4]thiadiazine;
6-(4-Azidophenyl)-3-(3,4-dimethoxyphenyl)-7H-[1,2,4]triazolo[3,4-b][1,3,4]thiadiazine;
6-(4-Acetamidophenyl)-3-(3,4-dimethoxyphenyl)-7H-[1,2,4]triazolo[3,4-b][1,3,4]thiadiazine;
6-(2,3-Dihydro-2-oxobenzo[d]oxazol-6-yl)-3-(2,4-Dimethoxyphenyl)-7H-[1,2,4]triazolo[3,4-b][1,3,4]thiadiazine;
6-(2,3-Dihydro-2-oxobenzo[d]oxazol-6-yl)-3-(3,5-dimethoxyphenyl)-7H-[1,2,4]triazolo[3,4-b][1,3,4]thiadiazine;
6-(2,3-Dihydro-2-oxobenzo[d]oxazol-6-yl)-3-(3,4-dimethoxyphenyl)-7H-[1,2,4]triazolo[3,4-b][1,3,4]thiadiazines;
3-(2-Methoxyphenyl)-6-(4-methoxyphenyl)-[1,2,4]triazolo[4,3-b][1,2,4]triazin-7(8H)-one;
3-(3,4,5-Trimethoxyphenyl)-6-(4-methylphenyl)-1H-[1,2,4]triazolo[4,3-b][1,2,4]triazin-7-one;
3-(2-M ethoxyphenyl)-6-(4-methylphenyl)-1H-[1,2,4]triazolo[4,3-b][1,2,4]triazin-7-one;
3-(2-Methoxyphenyl)-6-(2,4-dimethylphenyl)-7H-[1,2,4]triazolo[3,4-b][1,3,4]thiadiazine;
3-(2-Methoxyphenyl)-6-(3,4-dimethylphenyl)-7H-[1,2,4]triazolo[3,4-b][1,3,4]thiadiazine;
6-(2-Methoxy-4-methylphenyl)-3-(2-methoxyphenyl)-7H-[1,2,4]triazolo[3,4-b][1,3,4]thiadiazine;
6-(2,3,4-Trimethoxyphenyl)-3-(2-methoxyphenyl)-7H-[1,2,4]triazolo[3,4-b][1,3,4]thiadiazine;
6-(2,3-Dihydrobenzo[b][1,4]dioxin-6-yl)-3-(2-methoxyphenyl)-7H-[1,2,4]triazolo[3,4-b][1,3,4]thiadiazine;
6-(4-Isopropylphenyl)-3-(2-methoxyphenyl)-7H-[1,2,4]triazolo[3,4-b][1,3,4]thiadiazine;
6-(3-Methoxy-4-nitrophenyl)-3-(2-methoxyphenyl)-7H-[1,2,4]triazolo[3,4-b][1,3,4]thiadiazine;
3-(2-Methoxyphenyl)-6-(4-methoxyphenyl)-7H-[1,2,4]triazolo[3,4-b]-[1,3,4]thiadiazine;
6-(2,3-Dihydro-2-oxobenzo[d]oxazol-6-yl)-3-(2-methoxyphenyl)-7H-[1,2,4]triazolo[3,4-b][1,3,4]thiadiazine;
6-(4-(Trifluoromethyl)phenyl)-3-(2-methoxyphenyl)-7H-[1,2,4]triazolo[3,4-b][1,3,4]thiadiazine;
6-(5-Chloro-2-methoxy-4-methylphenyl)-3-(2-methoxyphenyl)-7H-[1,2,4]triazolo[3,4-b][1,3,4]thiadiazine;
3-(2-Methoxyphenyl)-6-(4-(trifluoromethoxy)phenyl)-7H-[1,2,4]triazolo[3,4-b][1,3,4]thiadiazine;
6-(4-tert-Butylphenyl)-3-(2-methylphenyl)-7H-[1,2,4]triazolo[3,4-b][1,3,4]thiadiazine;
6-(4-(N,N-Diethylaminosulfonyl)phenyl)-3-(2-methylphenyl)-7H-[1,2,4]triazolo[3,4-h][1,3,4]thiadiazine;
6-(4-Methyl-3-nitrophenyl)-3-(2-methylphenyl)-7H-[1,2,4]triazolo[3,4-b][1,3,4]thiadiazine;
3-(2-Methoxyphenyl)-6-(4-methyl-3-nitrophenyl)-7H-[1,2,4]triazolo[3,4-b][1,3,4]thiadiazine;
6-(4-Chloro-3-nitrophenyl)-3-(2-methoxyphenyl)-7H-[1,2,4]triazolo[3,4-b][1,3,4]thiadiazine;
6-(4-Acetamido-3-nitrophenyl)-3-(2-methoxyphenyl)-7H-[1,2,4]triazolo[3,4-b][1,3,4]thiadiazine;
6-(4-Amino-3-methoxyphenyl)-3-(2-methoxyphenyl)-7H-[1,2,4]triazolo[3,4-b][1,3,4]thiadiazine;
6-(3-Amino-4-methylphenyl)-3-(2-methoxyphenyl)-7H-[1,2,4]triazolo[3,4-b][1,3,4]thiadiazin;
6-(3-Amino-4-chlorophenyl)-3-(2-methoxyphenyl)-7H-[1,2,4]triazolo[3,4-b][1,3,4]thiadiazine;
6-(3,4-Diaminophenyl)-3-(2-methoxyphenyl)-7H-[1,2,4]triazolo[3,4-b][1,3,4]thiadiazine;
6-(3-Amino-4-methylphenyl)-3-(2-methylphenyl)-7H-[1,2,4]triazolo[3,4-b][1,3,4]thiadiazine;
3-(5-Chloro-2-methoxyphenyl)-6-(4-methylphenyl)-7H-[1,2,4]triazolo[3,4-b][1,3,4]thiadiazine;
3-(2-Methoxyphenyl)-6-(3-methoxylphenyl)-7H-[1,2,4]triazolo[3,4-b][1,3,4]thiadiazine;
3-(2-Methoxyphenyl)-6-(2-methoxylphenyl)-7H-[1,2,4]triazolo[3,4-b][1,3,4]thiadiazine;
3-(2,3-Dimethoxyphenyl)-6-(4-methylphenyl)-7H-[1,2,4]triazolo[3,4-b][1,3,4]thiadiazine;
6-(4-Aminophenyl)-3-(3,4,5-trimethoxyphenyl)-7H-[1,2,4]triazolo[3,4-b][1,3,4]thiadiazine;
6-(4-Dimethylaminophenyl)-3-(3,4,5-trimethoxyphenyl)-7H-[1,2,4]triazolo[3,4-b][1,3,4]thiadiazine;
3-(2-Fluorophenyl)-6-(4-methylphenyl)-7H-[1,2,4]triazolo[3,4-h][1,3,4]thiadiazine;
3-(2,5-Dimethoxyphenyl)-6-(4-methylphenyl)-7H-[1,2,4]triazolo[3,4-h][1,3,4]thiadiazine;
3-(2-Methylphenyl)-6-(4-methylphenyl)-7H-[1,2,4]triazolo[3,4-b][1,3,4]thiadiazine;
3-(2-Methylthiophenyl)-6-(4-methylphenyl)-7H-[1,2,4]triazolo[3,4-b][1,3,4]thiadiazine;
3-(2-Methylsulfinylphenyl)-6-(4-methylphenyl)-7H-[1,2,4]triazolo[3,4-b][1,3,4]thiadiazine;
3-(2-Methylsulfonylphenyl)-6-(4-methylphenyl)-7H-[1,2,4]triazolo[3,4-b][1,3,4]thiadiazine;
3-(2-Aminophenyl)-6-(4-methylphenyl)-7H-[1,2,4]triazolo[3,4-b][1,3,4]thiadiazine;
6-(4-Aminophenyl)-3-(2-methoxyphenyl)-7H-[1,2,4]triazolo[3,4-b][1,3,4]thiadiazine;
6-(4-Azidophenyl)-3-(2-methoxyphenyl)-7H-[1,2,4]triazolo[3,4-b][1,3,4]thiadiazine;
6-(4-dimethylaminophenyl)-3-(2-methoxyphenyl)-7H-[1,2,4]triazolo[3,4-b][1,3,4]thiadiazine;
6-(4-Amino-3,5-dibromophenyl)-3-(2-methoxyphenyl)-7H-[1,2,4]triazolo[3,4-h][1,3,4]thiadiazine;
6-(4-Amino-3,5-diiodophenyl)-3-(2-methoxyphenyl)-7H-[1,2,4]triazolo[3,4-b][1,3,4]thiadiazine;
6-(4-Isopropylaminophenyl)-3-(2-methoxyphenyl)-7H-[1,2,4]triazolo[3,4-b][1,3,4]thiadiazine;
3-(4-Chloro-2-methylphenyl)-6-(4-methylphenyl)-7H-[1,2,4]triazolo[3,4-b][1,3,4]thiadiazine;
3-(2-Methoxyphenyl)-6-(3-methylphenyl)-7H-[1,2,4]triazolo[3,4-b][1,3,4]thiadiazine;
3-(2-Methylfuran-3-yl)-6-(4-methylphenyl)-7H-[1,2,4]triazolo[3,4-b][1,3,4]thiadiazine;
3-(2-Methylaminophenyl)-6-(4-methylphenyl)-7H-[1,2,4]triazolo[3,4-b][1,3,4]thiadiazine;
6-(4-Methoxycarbonylphenyl)-3-(2-methoxyphenyl)-7H-[1,2,4]triazolo[3,4-b][1,3,4]thiadiazine;
3-(2-Methoxyphenyl)-6-(1-methyl-1H-pyrrol-3-yl)-7H-[1,2,4]triazolo[3,4-b][1,3,4]thiadiazine;
6-(4-Cyanophenyl)-3-(2-methoxyphenyl)-7H-[1,2,4]triazolo[3,4-b][1,3,4]thiadiazine;
5,6-Dihydro-3-(2-methoxyphenyl)-6-(4-methylphenyl)-7H-[1,2,4]triazolo[3,4-b][1,3,4]thiadiazine;
6-(4-Carboxylphenyl)-3-(2-methoxyphenyl)-7H-[1,2,4]triazolo[3,4-b][1,3,4]thiadiazine;
3-(4-Chloro-2-methylphenyl)-6-(4-methyl-3-nitrophenyl)-7H-[1,2,4]triazolo[3,4-b][1,3,4]thiadiazine;
6-(3-Amino-4-methylphenyl)-3-(4-chloro-2-methylphenyl)-7H-[1,2,4]triazolo[3,4-b][1,3,4]thiadiazine;
6-(4-Methylphenyl)-3-(1H-pyrrol-2-yl)-7H-[1,2,4]triazolo[3,4-b][1,3,4]thiadiazine;
3-(4-Methyl-1,2,3-thiadiazol-5-yl)-6-(4-methylphenyl)-7H-[1,2,4]triazolo[3,4-b][1,3,4]thiadiazine;
3-(1-Methyl-1H-pyrrol-2-yl)-6-(4-methylphenyl)-7H-[1,2,4]triazolo[3,4-b][1,3,4]thiadiazine;
3-(1-Methyl-4-nitro-1H-pyrrol-2-yl)-6-(4-methylphenyl)-7H-[1,2,4]triazolo[3,4-b][1,3,4]thiadiazine;
6-(7-Bromoindolin-5-yl)-3-(2-methoxyphenyl)-7H-[1,2,4]triazolo[3,4-b][1,3,4]thiadiazine;
3-(4-Chloro-2-methoxyphenyl)-6-(4-methylphenyl)-7H-[1,2,4]triazolo[3,4-b][1,3,4]thiadiazine;
3-(1-Methyl-4-amino-1H-pyrrol-2-yl)-6-(4-methylphenyl)-7H-[1,2,4]triazolo[3,4-b][1,3,4]thiadiazine;
3-(2-Methyl-imidazo[1,2-a]pyridin-3-yl)-6-(4-methylphenyl)-7H-[1,2,4]triazolo[3,4-b][1,3,4]thiadiazine;
or a pharmaceutically acceptable salt or prodrug thereof

30. A pharmaceutical composition comprising a pharmaceutically acceptable carrier and a compound of claim 29.

31. The pharmaceutical composition of claim 30, further comprising at least one known cancer chemotherapeutic agent, or a pharmaceutically acceptable salt of said agent.

32. The pharmaceutical composition of claim 30, further comprising at least one compound selected from the group consisting of busulfan, cis-platin, mitomycin C, carboplatin, colchicine, vinblastine, paclitaxel, docetaxel, camptothecin, topotecan, doxorubicin, etoposide, 5-azacytidine, 5-fluorouracil, methotrexate, 5-fluoro-2′-deoxy-uridine, ara-C, hydroxyurea, thioguanine, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen, Herceptin®, Rituxan®, arsenic trioxide, gemcitabine, doxazosin, terazosin, tamsulosin, CB-64D, CB-184, haloperidol, lovastatin, simvastatin, pravastatin, fluvastatin, atorvastatin, cerivastatin, amprenavir, abacavir, CGP-73547, CGP-61755, DMP-450, indinavir, nelfinavir, tipranavir, ritonavir, saquinavir, ABT-378, AG 1776, BMS-232,632, bexarotene, tretinoin, 13-cis-retinoic acid, 9-cis-retinoic acid, α-difluoromethylomithine, ILX23-7553, fenretinide, N-4-carboxyphenyl retinamide, lactacystin, MG-132, PS-341, Gleevec®, ZD1839 (Iressa), SH268, genistein, CEP2563, SU6668, SU11248, EMD121974, R115777, SCH66336, L-778,123, BAL9611, TAN-1813, flavopiridol, UCN-01, roscovitine, olomoucine, celecoxib, valecoxib, rofecoxib and alanosine.
